SEQUENCE LISTING (54 sequences)

SEQ ID NO: 1 - protein, 414 aa, synthetic construct
MSSPDAGYASDDQSQTQSALPAVMAGLGPCPWAESLSPIGDMKVKGEAPANSGAPAGAAGRAKGESRIRRPMNAFMVWAKDERKRLAQQNPDLHNAELSKMLGKSWKALTFAEKRPFNEEAVRLRVQHMQDHPNYKYRPRRRKQVKRLKRVEGGFLHGLAEPQAAALGPEGGRVAMDGLGLQFPEQGFPAGPPLLPPHMGGHYRDCQSLGAPPLDGYPLPTPDTSPLDGVDPDPAFFAAPMPGDCPAAGTYSYAQVSDYAGPPEPPAGPMHPRLGPEPAGPSIPGLLAPPSALHVYYGAMGSPGAGGGRGFQMQPQHQHQHQHQHHPPGPGQPSPPPEALPCRDGTDPSQPAELLGEVDRTEFEQYLHFVCKPEMGLPYQGHDSGVNLPDSHGAISSVVSDASSAVYYCNYPDV

SEQ ID NO: 2 - protein, 293 aa, synthetic construct
MSRIRRPMNAFMVWAKDERKRLAQQNPDLHNAELSKMLGKSWKALTFAEKRPFNEEAVRLRVQHMQDHPNYKYRPRRRKQVKRMKRVEGGFLHALVEPQAGALGPEGGRVAMDGLGLPFPEPGYPAGPPLMSPHMGPHYRDCQGLGAPALDGYPLPTPDTSPLDGVEQDPAFFAAPLPGDCPAAGTYTYAPVSDYAVSVEPPAGPMRVGPDPSGPAMPGILAPPSALHLYYGAMGSPAASAGRGFHAQPQQPLQPQAELLGEVDRTEFEQYLPFVYKPEMGLPYQGHDCGVNL

SEQ ID NO: 3 - DNA, 882 bp, synthetic construct
  source 1..882: mol_type = other DNA
atgtctcgcatccggcggccgatgaacgcctttatggtgtgggccaaagacgaacgcaagcggttggcacagcagaacccagatctgcacaacgcagagctaagcaagatgctaggcaagtcttggaaggcgttgacctttgcagagaagcggcccttcaatgaagaggccgtgcggctgcgcgtgcagcatatgcaggaccaccccaactacaagtaccggccgcggcggcgcaagcaggtgaagcgcatgaagcgggtggagggaggcttcctgcacgctctcgtcgagccccaggccggcgcgcttggtcccgagggcggccgcgtggccatggatggcctgggtctgcctttcccggagccgggctatccggccggtcctccgctgatgtctccgcacatgggcccccactatcgggactgccagggactgggcgctcccgcgctcgacggctaccctctgcccactccggacacatccccgctggatggcgtggagcaggacccggctttctttgcagccccgctgccaggggactgcccggcggccggcacctacacttacgctccagtctcggactatgcagtgtccgtagagccgcccgctggccccatgcgagtggggccggacccctcgggccctgcgatgccggggatcctggcgccccccagcgctctgcacctgtactacggcgcgatgggctcgcccgccgcaagtgcggggcgcggtttccacgcgcaaccccagcagccgctgcaaccgcaggcagagctcctaggggaggtggaccgcacggaattcgaacagtatctgccctttgtgtataagcccgagatgggtcttccctaccagggacacgactgcggagtgaacctctaa

SEQ ID NO: 4 - protein, 79 aa, synthetic construct
SRIRRPMNAFMVWAKDERKRLAQQNPDLHNAELSKMLGKSWKALTFAEKRPFNEEAVRLRVQHMQDHPNYKYRPRRRKQ

SEQ ID NO: 5 - protein, 414 aa, synthetic construct
MSSPDAGYASDDQSQTQSALPAVMAGLGPCPWAESLSPIGDMKVKGEAPANSGAPAGAAGRAKGESRIRRPMNAFMVWAKDERKRLAQQNPDLHNAELSKMLGKSWKALTLAEKRPFVEEAARLRVQHMQDHPNYKYRPRRRKQVKRLKRVEGGFLHGLAEPQAAALGPEGGRVAMDGLGLQFPEQGFPAGPPLLPPHMGGHYRDCQSLGAPPLDGYPLPTPDTSPLDGVDPDPAFFAAPMPGDCPAAGTYSYAQVSDYAGPPEPPAGPMHPRLGPEPAGPSIPGLLAPPSALHVYYGAMGSPGAGGGRGFQMQPQHQHQHQHQHHPPGPGQPSPPPEALPCRDGTDPSQPAELLGEVDRTEFEQYLHFVCKPEMGLPYQGHDSGVNLPDSHGAISSVVSDASSAVYYCNYPDV

SEQ ID NO: 6 - protein, 414 aa, synthetic construct
MSSPDAGYASDDQSQTQSALPAVMAGLGPCPWAESLSPIGDMKVKGEAPANSGAPAGAAGRAKGESRIRRPMNAFMVWAKDERKRLAQQNPDLHNAELSKMLGKSWKALTLAEKRPFVEEARRLRVQHMQDHPNYKYRPRRRKQVKRLKRVEGGFLHGLAEPQAAALGPEGGRVAMDGLGLQFPEQGFPAGPPLLPPHMGGHYRDCQSLGAPPLDGYPLPTPDTSPLDGVDPDPAFFAAPMPGDCPAAGTYSYAQVSDYAGPPEPPAGPMHPRLGPEPAGPSIPGLLAPPSALHVYYGAMGSPGAGGGRGFQMQPQHQHQHQHQHHPPGPGQPSPPPEALPCRDGTDPSQPAELLGEVDRTEFEQYLHFVCKPEMGLPYQGHDSGVNLPDSHGAISSVVSDASSAVYYCNYPDV

SEQ ID NO: 7 - protein, 414 aa, synthetic construct
MSSPDAGYASDDQSQTQSALPAVMAGLGPCPWAESLSPIGDMKVKGEAPANSGAPAGAAGRAKGESRIRRPMNAFMVWAKDERKRLAQQNPDLHNAELSKMLGKSWKALTLAEKRPFVEEANRLRVQHMQDHPNYKYRPRRRKQVKRLKRVEGGFLHGLAEPQAAALGPEGGRVAMDGLGLQFPEQGFPAGPPLLPPHMGGHYRDCQSLGAPPLDGYPLPTPDTSPLDGVDPDPAFFAAPMPGDCPAAGTYSYAQVSDYAGPPEPPAGPMHPRLGPEPAGPSIPGLLAPPSALHVYYGAMGSPGAGGGRGFQMQPQHQHQHQHQHHPPGPGQPSPPPEALPCRDGTDPSQPAELLGEVDRTEFEQYLHFVCKPEMGLPYQGHDSGVNLPDSHGAISSVVSDASSAVYYCNYPDV

SEQ ID NO: 8 - protein, 414 aa, synthetic construct
MSSPDAGYASDDQSQTQSALPAVMAGLGPCPWAESLSPIGDMKVKGEAPANSGAPAGAAGRAKGESRIRRPMNAFMVWAKDERKRLAQQNPDLHNAELSKMLGKSWKALTLAEKRPFVEEACRLRVQHMQDHPNYKYRPRRRKQVKRLKRVEGGFLHGLAEPQAAALGPEGGRVAMDGLGLQFPEQGFPAGPPLLPPHMGGHYRDCQSLGAPPLDGYPLPTPDTSPLDGVDPDPAFFAAPMPGDCPAAGTYSYAQVSDYAGPPEPPAGPMHPRLGPEPAGPSIPGLLAPPSALHVYYGAMGSPGAGGGRGFQMQPQHQHQHQHQHHPPGPGQPSPPPEALPCRDGTDPSQPAELLGEVDRTEFEQYLHFVCKPEMGLPYQGHDSGVNLPDSHGAISSVVSDASSAVYYCNYPDV

SEQ ID NO: 9 - protein, 414 aa, synthetic construct
MSSPDAGYASDDQSQTQSALPAVMAGLGPCPWAESLSPIGDMKVKGEAPANSGAPAGAAGRAKGESRIRRPMNAFMVWAKDERKRLAQQNPDLHNAELSKMLGKSWKALTLAEKRPFVEEAQRLRVQHMQDHPNYKYRPRRRKQVKRLKRVEGGFLHGLAEPQAAALGPEGGRVAMDGLGLQFPEQGFPAGPPLLPPHMGGHYRDCQSLGAPPLDGYPLPTPDTSPLDGVDPDPAFFAAPMPGDCPAAGTYSYAQVSDYAGPPEPPAGPMHPRLGPEPAGPSIPGLLAPPSALHVYYGAMGSPGAGGGRGFQMQPQHQHQHQHQHHPPGPGQPSPPPEALPCRDGTDPSQPAELLGEVDRTEFEQYLHFVCKPEMGLPYQGHDSGVNLPDSHGAISSVVSDASSAVYYCNYPDV

SEQ ID NO: 10 - protein, 414 aa, synthetic construct
MSSPDAGYASDDQSQTQSALPAVMAGLGPCPWAESLSPIGDMKVKGEAPANSGAPAGAAGRAKGESRIRRPMNAFMVWAKDERKRLAQQNPDLHNAELSKMLGKSWKALTLAEKRPFVEEAGRLRVQHMQDHPNYKYRPRRRKQVKRLKRVEGGFLHGLAEPQAAALGPEGGRVAMDGLGLQFPEQGFPAGPPLLPPHMGGHYRDCQSLGAPPLDGYPLPTPDTSPLDGVDPDPAFFAAPMPGDCPAAGTYSYAQVSDYAGPPEPPAGPMHPRLGPEPAGPSIPGLLAPPSALHVYYGAMGSPGAGGGRGFQMQPQHQHQHQHQHHPPGPGQPSPPPEALPCRDGTDPSQPAELLGEVDRTEFEQYLHFVCKPEMGLPYQGHDSGVNLPDSHGAISSVVSDASSAVYYCNYPDV

SEQ ID NO: 11 - protein, 414 aa, synthetic construct
MSSPDAGYASDDQSQTQSALPAVMAGLGPCPWAESLSPIGDMKVKGEAPANSGAPAGAAGRAKGESRIRRPMNAFMVWAKDERKRLAQQNPDLHNAELSKMLGKSWKALTLAEKRPFVEEAHRLRVQHMQDHPNYKYRPRRRKQVKRLKRVEGGFLHGLAEPQAAALGPEGGRVAMDGLGLQFPEQGFPAGPPLLPPHMGGHYRDCQSLGAPPLDGYPLPTPDTSPLDGVDPDPAFFAAPMPGDCPAAGTYSYAQVSDYAGPPEPPAGPMHPRLGPEPAGPSIPGLLAPPSALHVYYGAMGSPGAGGGRGFQMQPQHQHQHQHQHHPPGPGQPSPPPEALPCRDGTDPSQPAELLGEVDRTEFEQYLHFVCKPEMGLPYQGHDSGVNLPDSHGAISSVVSDASSAVYYCNYPDV

SEQ ID NO: 12 - protein, 414 aa, synthetic construct
MSSPDAGYASDDQSQTQSALPAVMAGLGPCPWAESLSPIGDMKVKGEAPANSGAPAGAAGRAKGESRIRRPMNAFMVWAKDERKRLAQQNPDLHNAELSKMLGKSWKALTLAEKRPFVEEAIRLRVQHMQDHPNYKYRPRRRKQVKRLKRVEGGFLHGLAEPQAAALGPEGGRVAMDGLGLQFPEQGFPAGPPLLPPHMGGHYRDCQSLGAPPLDGYPLPTPDTSPLDGVDPDPAFFAAPMPGDCPAAGTYSYAQVSDYAGPPEPPAGPMHPRLGPEPAGPSIPGLLAPPSALHVYYGAMGSPGAGGGRGFQMQPQHQHQHQHQHHPPGPGQPSPPPEALPCRDGTDPSQPAELLGEVDRTEFEQYLHFVCKPEMGLPYQGHDSGVNLPDSHGAISSVVSDASSAVYYCNYPDV

SEQ ID NO: 13 - protein, 414 aa, synthetic construct
MSSPDAGYASDDQSQTQSALPAVMAGLGPCPWAESLSPIGDMKVKGEAPANSGAPAGAAGRAKGESRIRRPMNAFMVWAKDERKRLAQQNPDLHNAELSKMLGKSWKALTLAEKRPFVEEALRLRVQHMQDHPNYKYRPRRRKQVKRLKRVEGGFLHGLAEPQAAALGPEGGRVAMDGLGLQFPEQGFPAGPPLLPPHMGGHYRDCQSLGAPPLDGYPLPTPDTSPLDGVDPDPAFFAAPMPGDCPAAGTYSYAQVSDYAGPPEPPAGPMHPRLGPEPAGPSIPGLLAPPSALHVYYGAMGSPGAGGGRGFQMQPQHQHQHQHQHHPPGPGQPSPPPEALPCRDGTDPSQPAELLGEVDRTEFEQYLHFVCKPEMGLPYQGHDSGVNLPDSHGAISSVVSDASSAVYYCNYPDV

SEQ ID NO: 14 - protein, 414 aa, synthetic construct
MSSPDAGYASDDQSQTQSALPAVMAGLGPCPWAESLSPIGDMKVKGEAPANSGAPAGAAGRAKGESRIRRPMNAFMVWAKDERKRLAQQNPDLHNAELSKMLGKSWKALTLAEKRPFVEEAKRLRVQHMQDHPNYKYRPRRRKQVKRLKRVEGGFLHGLAEPQAAALGPEGGRVAMDGLGLQFPEQGFPAGPPLLPPHMGGHYRDCQSLGAPPLDGYPLPTPDTSPLDGVDPDPAFFAAPMPGDCPAAGTYSYAQVSDYAGPPEPPAGPMHPRLGPEPAGPSIPGLLAPPSALHVYYGAMGSPGAGGGRGFQMQPQHQHQHQHQHHPPGPGQPSPPPEALPCRDGTDPSQPAELLGEVDRTEFEQYLHFVCKPEMGLPYQGHDSGVNLPDSHGAISSVVSDASSAVYYCNYPDV

SEQ ID NO: 15 - protein, 414 aa, synthetic construct
MSSPDAGYASDDQSQTQSALPAVMAGLGPCPWAESLSPIGDMKVKGEAPANSGAPAGAAGRAKGESRIRRPMNAFMVWAKDERKRLAQQNPDLHNAELSKMLGKSWKALTLAEKRPFVEEAMRLRVQHMQDHPNYKYRPRRRKQVKRLKRVEGGFLHGLAEPQAAALGPEGGRVAMDGLGLQFPEQGFPAGPPLLPPHMGGHYRDCQSLGAPPLDGYPLPTPDTSPLDGVDPDPAFFAAPMPGDCPAAGTYSYAQVSDYAGPPEPPAGPMHPRLGPEPAGPSIPGLLAPPSALHVYYGAMGSPGAGGGRGFQMQPQHQHQHQHQHHPPGPGQPSPPPEALPCRDGTDPSQPAELLGEVDRTEFEQYLHFVCKPEMGLPYQGHDSGVNLPDSHGAISSVVSDASSAVYYCNYPDV

SEQ ID NO: 16 - protein, 414 aa, synthetic construct
MSSPDAGYASDDQSQTQSALPAVMAGLGPCPWAESLSPIGDMKVKGEAPANSGAPAGAAGRAKGESRIRRPMNAFMVWAKDERKRLAQQNPDLHNAELSKMLGKSWKALTLAEKRPFVEEAFRLRVQHMQDHPNYKYRPRRRKQVKRLKRVEGGFLHGLAEPQAAALGPEGGRVAMDGLGLQFPEQGFPAGPPLLPPHMGGHYRDCQSLGAPPLDGYPLPTPDTSPLDGVDPDPAFFAAPMPGDCPAAGTYSYAQVSDYAGPPEPPAGPMHPRLGPEPAGPSIPGLLAPPSALHVYYGAMGSPGAGGGRGFQMQPQHQHQHQHQHHPPGPGQPSPPPEALPCRDGTDPSQPAELLGEVDRTEFEQYLHFVCKPEMGLPYQGHDSGVNLPDSHGAISSVVSDASSAVYYCNYPDV

SEQ ID NO: 17 - protein, 414 aa, synthetic construct
MSSPDAGYASDDQSQTQSALPAVMAGLGPCPWAESLSPIGDMKVKGEAPANSGAPAGAAGRAKGESRIRRPMNAFMVWAKDERKRLAQQNPDLHNAELSKMLGKSWKALTLAEKRPFVEEASRLRVQHMQDHPNYKYRPRRRKQVKRLKRVEGGFLHGLAEPQAAALGPEGGRVAMDGLGLQFPEQGFPAGPPLLPPHMGGHYRDCQSLGAPPLDGYPLPTPDTSPLDGVDPDPAFFAAPMPGDCPAAGTYSYAQVSDYAGPPEPPAGPMHPRLGPEPAGPSIPGLLAPPSALHVYYGAMGSPGAGGGRGFQMQPQHQHQHQHQHHPPGPGQPSPPPEALPCRDGTDPSQPAELLGEVDRTEFEQYLHFVCKPEMGLPYQGHDSGVNLPDSHGAISSVVSDASSAVYYCNYPDV

SEQ ID NO: 18 - protein, 414 aa, synthetic construct
MSSPDAGYASDDQSQTQSALPAVMAGLGPCPWAESLSPIGDMKVKGEAPANSGAPAGAAGRAKGESRIRRPMNAFMVWAKDERKRLAQQNPDLHNAELSKMLGKSWKALTLAEKRPFVEEATRLRVQHMQDHPNYKYRPRRRKQVKRLKRVEGGFLHGLAEPQAAALGPEGGRVAMDGLGLQFPEQGFPAGPPLLPPHMGGHYRDCQSLGAPPLDGYPLPTPDTSPLDGVDPDPAFFAAPMPGDCPAAGTYSYAQVSDYAGPPEPPAGPMHPRLGPEPAGPSIPGLLAPPSALHVYYGAMGSPGAGGGRGFQMQPQHQHQHQHQHHPPGPGQPSPPPEALPCRDGTDPSQPAELLGEVDRTEFEQYLHFVCKPEMGLPYQGHDSGVNLPDSHGAISSVVSDASSAVYYCNYPDV

SEQ ID NO: 19 - protein, 414 aa, synthetic construct
MSSPDAGYASDDQSQTQSALPAVMAGLGPCPWAESLSPIGDMKVKGEAPANSGAPAGAAGRAKGESRIRRPMNAFMVWAKDERKRLAQQNPDLHNAELSKMLGKSWKALTLAEKRPFVEEAWRLRVQHMQDHPNYKYRPRRRKQVKRLKRVEGGFLHGLAEPQAAALGPEGGRVAMDGLGLQFPEQGFPAGPPLLPPHMGGHYRDCQSLGAPPLDGYPLPTPDTSPLDGVDPDPAFFAAPMPGDCPAAGTYSYAQVSDYAGPPEPPAGPMHPRLGPEPAGPSIPGLLAPPSALHVYYGAMGSPGAGGGRGFQMQPQHQHQHQHQHHPPGPGQPSPPPEALPCRDGTDPSQPAELLGEVDRTEFEQYLHFVCKPEMGLPYQGHDSGVNLPDSHGAISSVVSDASSAVYYCNYPDV

SEQ ID NO: 20 - protein, 414 aa, synthetic construct
MSSPDAGYASDDQSQTQSALPAVMAGLGPCPWAESLSPIGDMKVKGEAPANSGAPAGAAGRAKGESRIRRPMNAFMVWAKDERKRLAQQNPDLHNAELSKMLGKSWKALTLAEKRPFVEEAYRLRVQHMQDHPNYKYRPRRRKQVKRLKRVEGGFLHGLAEPQAAALGPEGGRVAMDGLGLQFPEQGFPAGPPLLPPHMGGHYRDCQSLGAPPLDGYPLPTPDTSPLDGVDPDPAFFAAPMPGDCPAAGTYSYAQVSDYAGPPEPPAGPMHPRLGPEPAGPSIPGLLAPPSALHVYYGAMGSPGAGGGRGFQMQPQHQHQHQHQHHPPGPGQPSPPPEALPCRDGTDPSQPAELLGEVDRTEFEQYLHFVCKPEMGLPYQGHDSGVNLPDSHGAISSVVSDASSAVYYCNYPDV

SEQ ID NO: 21 - protein, 79 aa, synthetic construct
SRIRRPMNAFMVWAKDERKRLAQQNPDLHNAELSKMLGKSWKALTLAEKRPFVEEAARLRVQHMQDHPNYKYRPRRRKQ

SEQ ID NO: 22 - protein, 79 aa, synthetic construct
SRIRRPMNAFMVWAKDERKRLAQQNPDLHNAELSKMLGKSWKALTLAEKRPFVEEARRLRVQHMQDHPNYKYRPRRRKQ

SEQ ID NO: 23 - protein, 79 aa, synthetic construct
SRIRRPMNAFMVWAKDERKRLAQQNPDLHNAELSKMLGKSWKALTLAEKRPFVEEANRLRVQHMQDHPNYKYRPRRRKQ

SEQ ID NO: 24 - protein, 79 aa, synthetic construct
SRIRRPMNAFMVWAKDERKRLAQQNPDLHNAELSKMLGKSWKALTLAEKRPFVEEACRLRVQHMQDHPNYKYRPRRRKQ

SEQ ID NO: 25 - protein, 79 aa, synthetic construct
SRIRRPMNAFMVWAKDERKRLAQQNPDLHNAELSKMLGKSWKALTLAEKRPFVEEAQRLRVQHMQDHPNYKYRPRRRKQ

SEQ ID NO: 26 - protein, 79 aa, synthetic construct
SRIRRPMNAFMVWAKDERKRLAQQNPDLHNAELSKMLGKSWKALTLAEKRPFVEEAGRLRVQHMQDHPNYKYRPRRRKQ

SEQ ID NO: 27 - protein, 79 aa, synthetic construct
SRIRRPMNAFMVWAKDERKRLAQQNPDLHNAELSKMLGKSWKALTLAEKRPFVEEAHRLRVQHMQDHPNYKYRPRRRKQ

SEQ ID NO: 28 - protein, 79 aa, synthetic construct
SRIRRPMNAFMVWAKDERKRLAQQNPDLHNAELSKMLGKSWKALTLAEKRPFVEEAIRLRVQHMQDHPNYKYRPRRRKQ

SEQ ID NO: 29 - protein, 79 aa, synthetic construct
SRIRRPMNAFMVWAKDERKRLAQQNPDLHNAELSKMLGKSWKALTLAEKRPFVEEALRLRVQHMQDHPNYKYRPRRRKQ

SEQ ID NO: 30 - protein, 79 aa, synthetic construct
SRIRRPMNAFMVWAKDERKRLAQQNPDLHNAELSKMLGKSWKALTLAEKRPFVEEAKRLRVQHMQDHPNYKYRPRRRKQ

SEQ ID NO: 31 - protein, 79 aa, synthetic construct
SRIRRPMNAFMVWAKDERKRLAQQNPDLHNAELSKMLGKSWKALTLAEKRPFVEEAMRLRVQHMQDHPNYKYRPRRRKQ

SEQ ID NO: 32 - protein, 79 aa, synthetic construct
SRIRRPMNAFMVWAKDERKRLAQQNPDLHNAELSKMLGKSWKALTLAEKRPFVEEAFRLRVQHMQDHPNYKYRPRRRKQ

SEQ ID NO: 33 - protein, 79 aa, synthetic construct
SRIRRPMNAFMVWAKDERKRLAQQNPDLHNAELSKMLGKSWKALTLAEKRPFVEEASRLRVQHMQDHPNYKYRPRRRKQ

SEQ ID NO: 34 - protein, 79 aa, synthetic construct
SRIRRPMNAFMVWAKDERKRLAQQNPDLHNAELSKMLGKSWKALTLAEKRPFVEEATRLRVQHMQDHPNYKYRPRRRKQ

SEQ ID NO: 35 - protein, 79 aa, synthetic construct
SRIRRPMNAFMVWAKDERKRLAQQNPDLHNAELSKMLGKSWKALTLAEKRPFVEEAWRLRVQHMQDHPNYKYRPRRRKQ

SEQ ID NO: 36 - protein, 79 aa, synthetic construct
SRIRRPMNAFMVWAKDERKRLAQQNPDLHNAELSKMLGKSWKALTLAEKRPFVEEAYRLRVQHMQDHPNYKYRPRRRKQ

SEQ ID NO: 37 - protein, 79 aa, synthetic construct
SRIRRPMNAFMVWAKDERKRLAQQNPDLHNAELSKMLGKSWKALTLAEKRPFVEEAVRLRVQHMQDHPNYKYRPRRRKQ

SEQ ID NO: 38 - protein, 293 aa, synthetic construct
MSRIRRPMNAFMVWAKDERKRLAQQNPDLHNAELSKMLGKSWKALTLAEKRPFVEEAARLRVQHMQDHPNYKYRPRRRKQVKRMKRVEGGFLHALVEPQAGALGPEGGRVAMDGLGLPFPEPGYPAGPPLMSPHMGPHYRDCQGLGAPALDGYPLPTPDTSPLDGVEQDPAFFAAPLPGDCPAAGTYTYAPVSDYAVSVEPPAGPMRVGPDPSGPAMPGILAPPSALHLYYGAMGSPAASAGRGFHAQPQQPLQPQAELLGEVDRTEFEQYLPFVYKPEMGLPYQGHDCGVNL

SEQ ID NO: 39 - protein, 293 aa, synthetic construct
MSRIRRPMNAFMVWAKDERKRLAQQNPDLHNAELSKMLGKSWKALTLAEKRPFVEEARRLRVQHMQDHPNYKYRPRRRKQVKRMKRVEGGFLHALVEPQAGALGPEGGRVAMDGLGLPFPEPGYPAGPPLMSPHMGPHYRDCQGLGAPALDGYPLPTPDTSPLDGVEQDPAFFAAPLPGDCPAAGTYTYAPVSDYAVSVEPPAGPMRVGPDPSGPAMPGILAPPSALHLYYGAMGSPAASAGRGFHAQPQQPLQPQAELLGEVDRTEFEQYLPFVYKPEMGLPYQGHDCGVNL

SEQ ID NO: 40 - protein, 293 aa, synthetic construct
MSRIRRPMNAFMVWAKDERKRLAQQNPDLHNAELSKMLGKSWKALTLAEKRPFVEEANRLRVQHMQDHPNYKYRPRRRKQVKRMKRVEGGFLHALVEPQAGALGPEGGRVAMDGLGLPFPEPGYPAGPPLMSPHMGPHYRDCQGLGAPALDGYPLPTPDTSPLDGVEQDPAFFAAPLPGDCPAAGTYTYAPVSDYAVSVEPPAGPMRVGPDPSGPAMPGILAPPSALHLYYGAMGSPAASAGRGFHAQPQQPLQPQAELLGEVDRTEFEQYLPFVYKPEMGLPYQGHDCGVNL

SEQ ID NO: 41 - protein, 293 aa, synthetic construct
MSRIRRPMNAFMVWAKDERKRLAQQNPDLHNAELSKMLGKSWKALTLAEKRPFVEEACRLRVQHMQDHPNYKYRPRRRKQVKRMKRVEGGFLHALVEPQAGALGPEGGRVAMDGLGLPFPEPGYPAGPPLMSPHMGPHYRDCQGLGAPALDGYPLPTPDTSPLDGVEQDPAFFAAPLPGDCPAAGTYTYAPVSDYAVSVEPPAGPMRVGPDPSGPAMPGILAPPSALHLYYGAMGSPAASAGRGFHAQPQQPLQPQAELLGEVDRTEFEQYLPFVYKPEMGLPYQGHDCGVNL

SEQ ID NO: 42 - protein, 293 aa, synthetic construct
MSRIRRPMNAFMVWAKDERKRLAQQNPDLHNAELSKMLGKSWKALTLAEKRPFVEEAQRLRVQHMQDHPNYKYRPRRRKQVKRMKRVEGGFLHALVEPQAGALGPEGGRVAMDGLGLPFPEPGYPAGPPLMSPHMGPHYRDCQGLGAPALDGYPLPTPDTSPLDGVEQDPAFFAAPLPGDCPAAGTYTYAPVSDYAVSVEPPAGPMRVGPDPSGPAMPGILAPPSALHLYYGAMGSPAASAGRGFHAQPQQPLQPQAELLGEVDRTEFEQYLPFVYKPEMGLPYQGHDCGVNL

SEQ ID NO: 43 - protein, 293 aa, synthetic construct
MSRIRRPMNAFMVWAKDERKRLAQQNPDLHNAELSKMLGKSWKALTLAEKRPFVEEAGRLRVQHMQDHPNYKYRPRRRKQVKRMKRVEGGFLHALVEPQAGALGPEGGRVAMDGLGLPFPEPGYPAGPPLMSPHMGPHYRDCQGLGAPALDGYPLPTPDTSPLDGVEQDPAFFAAPLPGDCPAAGTYTYAPVSDYAVSVEPPAGPMRVGPDPSGPAMPGILAPPSALHLYYGAMGSPAASAGRGFHAQPQQPLQPQAELLGEVDRTEFEQYLPFVYKPEMGLPYQGHDCGVNL

SEQ ID NO: 44 - protein, 293 aa, synthetic construct
MSRIRRPMNAFMVWAKDERKRLAQQNPDLHNAELSKMLGKSWKALTLAEKRPFVEEAHRLRVQHMQDHPNYKYRPRRRKQVKRMKRVEGGFLHALVEPQAGALGPEGGRVAMDGLGLPFPEPGYPAGPPLMSPHMGPHYRDCQGLGAPALDGYPLPTPDTSPLDGVEQDPAFFAAPLPGDCPAAGTYTYAPVSDYAVSVEPPAGPMRVGPDPSGPAMPGILAPPSALHLYYGAMGSPAASAGRGFHAQPQQPLQPQAELLGEVDRTEFEQYLPFVYKPEMGLPYQGHDCGVNL

SEQ ID NO: 45 - protein, 293 aa, synthetic construct
MSRIRRPMNAFMVWAKDERKRLAQQNPDLHNAELSKMLGKSWKALTLAEKRPFVEEAIRLRVQHMQDHPNYKYRPRRRKQVKRMKRVEGGFLHALVEPQAGALGPEGGRVAMDGLGLPFPEPGYPAGPPLMSPHMGPHYRDCQGLGAPALDGYPLPTPDTSPLDGVEQDPAFFAAPLPGDCPAAGTYTYAPVSDYAVSVEPPAGPMRVGPDPSGPAMPGILAPPSALHLYYGAMGSPAASAGRGFHAQPQQPLQPQAELLGEVDRTEFEQYLPFVYKPEMGLPYQGHDCGVNL

SEQ ID NO: 46 - protein, 293 aa, synthetic construct
MSRIRRPMNAFMVWAKDERKRLAQQNPDLHNAELSKMLGKSWKALTLAEKRPFVEEALRLRVQHMQDHPNYKYRPRRRKQVKRMKRVEGGFLHALVEPQAGALGPEGGRVAMDGLGLPFPEPGYPAGPPLMSPHMGPHYRDCQGLGAPALDGYPLPTPDTSPLDGVEQDPAFFAAPLPGDCPAAGTYTYAPVSDYAVSVEPPAGPMRVGPDPSGPAMPGILAPPSALHLYYGAMGSPAASAGRGFHAQPQQPLQPQAELLGEVDRTEFEQYLPFVYKPEMGLPYQGHDCGVNL

SEQ ID NO: 47 - protein, 293 aa, synthetic construct
MSRIRRPMNAFMVWAKDERKRLAQQNPDLHNAELSKMLGKSWKALTLAEKRPFVEEAKRLRVQHMQDHPNYKYRPRRRKQVKRMKRVEGGFLHALVEPQAGALGPEGGRVAMDGLGLPFPEPGYPAGPPLMSPHMGPHYRDCQGLGAPALDGYPLPTPDTSPLDGVEQDPAFFAAPLPGDCPAAGTYTYAPVSDYAVSVEPPAGPMRVGPDPSGPAMPGILAPPSALHLYYGAMGSPAASAGRGFHAQPQQPLQPQAELLGEVDRTEFEQYLPFVYKPEMGLPYQGHDCGVNL

SEQ ID NO: 48 - protein, 293 aa, synthetic construct
MSRIRRPMNAFMVWAKDERKRLAQQNPDLHNAELSKMLGKSWKALTLAEKRPFVEEAMRLRVQHMQDHPNYKYRPRRRKQVKRMKRVEGGFLHALVEPQAGALGPEGGRVAMDGLGLPFPEPGYPAGPPLMSPHMGPHYRDCQGLGAPALDGYPLPTPDTSPLDGVEQDPAFFAAPLPGDCPAAGTYTYAPVSDYAVSVEPPAGPMRVGPDPSGPAMPGILAPPSALHLYYGAMGSPAASAGRGFHAQPQQPLQPQAELLGEVDRTEFEQYLPFVYKPEMGLPYQGHDCGVNL

SEQ ID NO: 49 - protein, 293 aa, synthetic construct
MSRIRRPMNAFMVWAKDERKRLAQQNPDLHNAELSKMLGKSWKALTLAEKRPFVEEAFRLRVQHMQDHPNYKYRPRRRKQVKRMKRVEGGFLHALVEPQAGALGPEGGRVAMDGLGLPFPEPGYPAGPPLMSPHMGPHYRDCQGLGAPALDGYPLPTPDTSPLDGVEQDPAFFAAPLPGDCPAAGTYTYAPVSDYAVSVEPPAGPMRVGPDPSGPAMPGILAPPSALHLYYGAMGSPAASAGRGFHAQPQQPLQPQAELLGEVDRTEFEQYLPFVYKPEMGLPYQGHDCGVNL

SEQ ID NO: 50 - protein, 293 aa, synthetic construct
MSRIRRPMNAFMVWAKDERKRLAQQNPDLHNAELSKMLGKSWKALTLAEKRPFVEEASRLRVQHMQDHPNYKYRPRRRKQVKRMKRVEGGFLHALVEPQAGALGPEGGRVAMDGLGLPFPEPGYPAGPPLMSPHMGPHYRDCQGLGAPALDGYPLPTPDTSPLDGVEQDPAFFAAPLPGDCPAAGTYTYAPVSDYAVSVEPPAGPMRVGPDPSGPAMPGILAPPSALHLYYGAMGSPAASAGRGFHAQPQQPLQPQAELLGEVDRTEFEQYLPFVYKPEMGLPYQGHDCGVNL

SEQ ID NO: 51 - protein, 293 aa, synthetic construct
MSRIRRPMNAFMVWAKDERKRLAQQNPDLHNAELSKMLGKSWKALTLAEKRPFVEEATRLRVQHMQDHPNYKYRPRRRKQVKRMKRVEGGFLHALVEPQAGALGPEGGRVAMDGLGLPFPEPGYPAGPPLMSPHMGPHYRDCQGLGAPALDGYPLPTPDTSPLDGVEQDPAFFAAPLPGDCPAAGTYTYAPVSDYAVSVEPPAGPMRVGPDPSGPAMPGILAPPSALHLYYGAMGSPAASAGRGFHAQPQQPLQPQAELLGEVDRTEFEQYLPFVYKPEMGLPYQGHDCGVNL

SEQ ID NO: 52 - protein, 293 aa, synthetic construct
MSRIRRPMNAFMVWAKDERKRLAQQNPDLHNAELSKMLGKSWKALTLAEKRPFVEEAWRLRVQHMQDHPNYKYRPRRRKQVKRMKRVEGGFLHALVEPQAGALGPEGGRVAMDGLGLPFPEPGYPAGPPLMSPHMGPHYRDCQGLGAPALDGYPLPTPDTSPLDGVEQDPAFFAAPLPGDCPAAGTYTYAPVSDYAVSVEPPAGPMRVGPDPSGPAMPGILAPPSALHLYYGAMGSPAASAGRGFHAQPQQPLQPQAELLGEVDRTEFEQYLPFVYKPEMGLPYQGHDCGVNL

SEQ ID NO: 53 - protein, 293 aa, synthetic construct
MSRIRRPMNAFMVWAKDERKRLAQQNPDLHNAELSKMLGKSWKALTLAEKRPFVEEAYRLRVQHMQDHPNYKYRPRRRKQVKRMKRVEGGFLHALVEPQAGALGPEGGRVAMDGLGLPFPEPGYPAGPPLMSPHMGPHYRDCQGLGAPALDGYPLPTPDTSPLDGVEQDPAFFAAPLPGDCPAAGTYTYAPVSDYAVSVEPPAGPMRVGPDPSGPAMPGILAPPSALHLYYGAMGSPAASAGRGFHAQPQQPLQPQAELLGEVDRTEFEQYLPFVYKPEMGLPYQGHDCGVNL

SEQ ID NO: 54 - protein, 293 aa, synthetic construct
MSRIRRPMNAFMVWAKDERKRLAQQNPDLHNAELSKMLGKSWKALTLAEKRPFVEEAVRLRVQHMQDHPNYKYRPRRRKQVKRMKRVEGGFLHALVEPQAGALGPEGGRVAMDGLGLPFPEPGYPAGPPLMSPHMGPHYRDCQGLGAPALDGYPLPTPDTSPLDGVEQDPAFFAAPLPGDCPAAGTYTYAPVSDYAVSVEPPAGPMRVGPDPSGPAMPGILAPPSALHLYYGAMGSPAASAGRGFHAQPQQPLQPQAELLGEVDRTEFEQYLPFVYKPEMGLPYQGHDCGVNL

(19) United States
(12) Patent Application Publication
(10) Pub. No.: US 2024/0084265 A1
(43) Pub. Date: Mar. 14, 2024

(54) METHODS TO CONVERT SOMATIC HUMAN CELL TO A TOTIPOTENCY-LIKE STATE WITH ENGINEERED SOX17

(71) Applicants: The University of Hong Kong, Hong Kong (CN); Centre for Translational Stem Cell Biology Limited, Hong Kong (CN)

(72) Inventors: Ralf Jauch, Hong Kong (CN); Haoqing Hu, Hong Kong (CN); Mingxi Weng, Hong Kong (CN); Hoi Hang Ho, Hong Kong (CN)

(21) Appl. No.: 18/453,112

(22) Filed: Aug. 21, 2023

Related U.S. Application Data
(60) Provisional application No. 63/373,441, filed on Aug. 24, 2022.

(51) Int. Cl. C12N 5/074; C12N 15/86

(52) U.S. Cl. CPC C12N 5/0696; C12N 15/86; C12N 2501/60; C12N 2501/603; C12N 2501/604; C12N 2501/606; C12N 2506/11; C12N 2506/1307

(57) ABSTRACT

Compositions of transcription factor cocktails including engineered SOX17 factor (eSOX17), together with one or more of OCT4, KLF4, and C-MYC, and vectors carrying one or more of these transcription factors suitable for delivery to donor somatic cells for generating induced expanded potential stem cells (iEPSCs) or induced neural stem cells (iNSCs) are provided. The disclosed compositions deliver reprograming of human somatic cells into a totipotency-like, or multipotency-like state with improved efficiency. Compositions of eSOX17-derived iEPSCs and iNSCs generated according to the described methods are also described. The methods engineer somatic cells to express one or more markers of pluripotency or neural stem cells. Methods of using iEPSCs or iNSCs in studying early human development and in cell therapy are also provided.

### CROSS-REFERENCE TO RELATED APPLICATIONS

This application claims benefit of and priority to U.S. Patent Application No. 63/373,441, filed on Aug. 24, 2022, the contents of which is hereby incorporated by reference herein in its entirety.

### REFERENCE TO SEQUENCE LISTING

The Sequence Listing submitted as a text file named “UHK_01200_ST26.txt,” created on Aug. 11, 2023, and having a size of 62,000 bytes is hereby incorporated by reference pursuant to 37 C.F.R. § 1.834(c)(1).

### FIELD OF THE INVENTION

The invention is generally directed to compositions and methods for reprogramming human somatic cells into cells with characteristics of totipotent stem cells, and more specifically to induced expanded potential stem cells (iEPSCs) and/or induced neural stem cells (iNSCs) derived from somatic cells and methods of use thereof.

### BACKGROUND OF THE INVENTION

Cell-fate conversion can be induced through forced expression of defined transcription factor cocktails in somatic cells (Takahashi K, et al., Cell. 2007;131(5):861-72; Takahashi and Yamanaka, Cell. 2006;126(4):663-76). Yamanaka factors, the four-factor transcription factor cocktail of OCT4, SOX2, KLF4 and C-MYC, have been reported to direct human and mouse induced pluripotent stem cell (iPSC) reprogramming. However, speed, efficiency, and reproducibility of pluripotent stem cells by reprogramming methods are still poor and pose great hurdle to translate somatic cell reprogramming in clinical studies.

Human embryonic stem cells (ESCs) are derived from pre-implantation embryos with self-renewal capacity in long term cultures and the potential to differentiation to all embryonic cell lineages (Thomson J A, et al., Science. 1998;282(5391):1145-7). However, human ESCs and iPSCs represent a post-implantation or ‘primed’ state that is much less efficient to generate extra-embryonic tissues that give rise to placenta or yolk sac. Hence, the early stages of human development and implantation cannot be easily studied with these cells.

Therefore, it is an object of the invention to provide improved compositions and methods of use thereof for reprogramming human somatic cells to induce totipotency-like features with improved efficiency, speed, and reproducibility.

It is another object of the invention to provide compositions and methods of cell models suitable for use in preclinical study of infertility-related diseases and early human development.

It is a further object of the invention to provide compositions and methods for providing in vitro drug screening platforms for development of therapeutic agents/compounds for the treatment of infertility-related diseases.

### SUMMARY OF THE INVENTION

Compositions and methods are disclosed for improving reprogramming of human somatic cells into totipotent-like cells. The disclosed methods overcome the inadequacy of prior art methods by adopting an engineered SOX17 transcription factor along with OCT4, KLF4 and C-MYC, allowing for improved reprogramming of human somatic cells. As demonstrated in the Examples, the disclosed methods are useful to reprogram human somatic cells, such as human adult dermal fibroblasts.

Typically, the methods for reprogramming human somatic cells into human induced expanded potential stem cells (iEPSCs) include one or more steps of (i) introducing exogenous transcription factors to the human somatic cells, wherein the exogenous transcription factors include an engineered SOX17 factor, and (ii) culturing the population in an expanded potential stem cell medium (EPSCM).

In some forms, the engineered SOX 17 factor (eSOX17) includes the amino acid sequence of any one of SEQ ID NO:1 or SEQ ID NO:5-20, or an amino acid sequence having at least 70%, 75%, 80%, 85%, 90%, 95%, or 99% sequence identity to SEQ ID NO:1 or SEQ ID NO:5-20, or a fragment variant thereof. In other forms, the engineered SOX 17 factor (eSOX17) includes the amino acid sequence of any one of SEQ ID NO:2 or SEQ ID NO:38-54, or an amino acid sequence having at least 70%, 75%, 80%, 85%, 90%, 95%, or 99% sequence identity to SEQ ID NO:2 or SEQ ID NO:38-54, or a fragment variant thereof. In other forms, the engineered SOX 17 factor (eSOX17) includes the amino acid sequence of any one of SEQ ID NO:4 or SEQ ID NO:21-37.

In preferred forms, the exogenous transcription factors include OCT4, KLF4, and C-MYC. Preferably, the exogenous transcription factors are encoded as nucleic acids. In some forms, the nucleic acids encoding the exogenous transcription factors are incorporated into a vector, for example, an integration-free system such as episomal vectors, minicircle vectors, integration-deficient lentivirus, and Sendai viral vectors, or an integration viral vector such as retrovirus, lentivirus, adenovirus, or herpes virus.

In some methods, the eSOX17 includes the amino acid sequence of SEQ ID NO:1-2, or SEQ ID NO:4-54, the exogenous transcription factors are OCT4, KLF4 and C-MYC; and the cells are eSOX17-derived induced expanded potential stem cells (iEPSCs). In some methods, the eSOX17 includes the amino acid sequence of SEQ ID NO:1-2, or SEQ ID NO:4-54, the exogenous transcription factor is C-MYC; and the cells are induced neural stem cells (iNSCs).

Generally, the EPSCM includes one or more of a RAS-ERK inhibitor, a Src Kinase family (SFK) inhibitor, a GSK3 inhibitor, a Wnt inhibitor, a p38 inhibitor, and a JNK inhibitor (Gao, et al., Nature Cell Biology. 2019;21(6):687-699). The methods are effective to provide EPSC-like colonies are produced in less than 6 days after culturing in EPSCM. In some forms, the methods further include the step of picking EPSC-like colonies to generate single cell iEPSC clone. In other forms, the methods further include adapting iEPSCs to feeder-free condition. In further forms, the methods expand iEPSCs for more than 20 passages, for example, for up to 25 passages in cell culture. In some forms, the methods further include differentiating iEPSCs into trophoblasts and/or blastoids.

The iEPSCs can be obtained by induced pluripotent cells, or partially or completely differentiated cells obtained from a mammal such as any mammal (e.g., bovine, ovine, porcine, canine, feline, equine, primate), preferably a human. Sources include cells of hematological origin, skin derived cells, adipose cells, epithelial cells, endothelial cells, cells of mesenchymal origin, parenchymal cells, neurological cells, and connective tissue cells. In one form, the human somatic cells are dermal fibroblasts. In another form, the human somatic cells are peripheral blood cells. In some forms, the human somatic cells are reprogramming-resistant cells such as high passage senescent fibroblasts. In other forms, the human somatic cells are reprogramming-resistant cells where OCT4, KLF4, and C-MYC in combination with SOX2 fails to induce pluripotency or totipotency-like states.

Also provided are human totipotent-like and/or multi-potent stem cells obtained by the disclosed methods, characterized in that they express at least the core pluripotency transcriptional factors engineered SOX17, OCT4, KLF4, and C-MYC. In some forms, the disclosed human totipotent-like stem cells are cryopreserved for later use. In other forms, the eSOX17-derived cells are induced neural stem cells (iNSCs). Typically, the iNSCs are tri-potent cells.

Methods of using the disclosed human totipotent-like stem cells are also described. In some forms, the iEPSCs are suitable for use in study of reproductive human disorders such as preeclampsia as cell models for implantation and early human development without embryo destruction. In further forms, the iEPSCs provide therapeutic use in ameliorating human reproductive disorders such as preeclampsia or uterine disorders such as endometriosis.

Compositions of engineered SOX17 factors (eSOX17) are also provided. Typically, the compositions of eSOX17 include the amino acid sequence of any one of SEQ ID NO:1-2 or SEQ ID NO:4-54, or an amino acid sequence having at least 70%, 75%, 80%, 85%, 90%, 95%, or 99% sequence identity to SEQ ID NO:1-2, or SEQ ID NO:4-54, or a fragment variant thereof. In some forms, compositions of eSOX17 include a nucleic acid encoding a polypeptide the amino acid sequence of any one of SEQ ID NO:1-2 or SEQ ID NO:4-54, or an amino acid sequence having at least 70%, 75%, 80%, 85%, 90%, 95%, or 99% sequence identity to SEQ ID NO:1-2, or SEQ ID NO:4-54, or a fragment variant thereof. Lentiviral vectors expressing or encoding an eSOX17 including the amino acid sequence of any one of SEQ ID NO:1-2 or SEQ ID NO:4-54, or an amino acid sequence having at least 70%, 75%, 80%, 85%, 90%, 95%, or 99% sequence identity to SEQ ID NO:1-2, or SEQ ID NO:4-54, or a fragment variant thereof are also provided.

### BRIEF DESCRIPTION OF THE DRAWINGS

FIG. 1A is a schematic outlining steps involved in human EPSC somatic reprogramming; FIG. 1B shows primary iEPSC colonies grown on MEF-feeders; FIG. 1C shows Alkaline Phosphatase (AP) staining of reprogrammed cells at day 24; FIG. 1D shows EPSC colony count during reprogramming over a period of 17 days in OKMS2 and OKMSOX17FNV; FIG. 1E is a graph of reprogramming efficiency (0-3%) at day 16 in iEPSC reprogramed by SOX17FNV from different passages of human fibroblasts (HDF P6 and senescent HDF P17), respectively.

FIGS. 2A-2D are photograph images of iEPSC colonies (SOX2-derived iEPSCs and SOX17FNV-derived iEPSCs, respectively) grown on each of feeder cells (FIGS. 2A-2B) and Matrigel (FIGS. 2C-2D), respectively. FIGS. 2E-2O are bar graphs of expression of pluripotency and lineage differentiation marker genes relative to GADPH expression for each of OCT4 (FIG. 2E); NANOG (FIG. 2F); SOX2 (FIG. 2G); LIN28 (FIG. 2H); T (FIG. 2I); HNF4A (FIG. 2J); HAND1 (FIG. 2K); FOXA2 (FIG. 2L); PAX6 (FIG. 2M); GBX2 (FIG. 2N); and DES (FIG. 2O), respectively, in each of human SOX2-derived iEPSC clones and SOX17FNV-derived iEPSC clones under either MEF feeder condition or feeder-free condition (Matrigel), respectively.

FIGS. 3A-3E are bar graphs showing relative mRNA levels of trophoblast-related marker genes including CDX2 (FIG. 3A); GATA3 (FIG. 3B); KRT8 (FIG. 3C); KRT18 (FIG. 3D); and HAND1 (FIG. 3E) in the established feeder-free SOX2-derived iEPSCs and SOX17FNV-derived iEPSCs with or without TGFP inhibitor (SB431542) treatment, respectively.

FIGS. 4A-4E are graphs showing expression of blastocyst and trophoblast related marker genes, including CDX2 (FIG. 4A); GATA3 (FIG. 4B); KRT8 (FIG. 4C); KRT18 (FIG. 4D); and TRAP2C (FIG. 4E) in each of parental EPSC and in iEPSCs upon spontaneous differentiation (Diff. rep. 1 and Diff. rep. 2), respectively. FIGS. 4F-4G are images showing the blastocyst-like structure generated from iEPSCs upon spontaneous differentiation under light microscopy (FIG. 4F); and staining using DAPI (FIG. 4G), respectively. FIG. 4H is a graph showing diameters of induced blastoids (0-400 for n=24) under PALLY method using human iEPSCs including each of feeder-free EPSC clones: SOX2-derived clones (S2-1, S2-2) and SOX17FNV-derived clones (FNV3 and FNV8), respectively, with cell seeding number 200 or 400 as indicated.

FIG. 5A is a schematic outlining steps in the generation of iNSCs with eSOX17FNV from human blood cells, showing reprogramming of human erythroid progenitor cells (EPCs) into hiNSCs. FIG. 5B is a graph of PC analysis of global gene expression profiles determined by RNA-seq, showing PC2 (31.2%) over PC1 (40.2%) for each of the indicated samples. FIG. 5C is a graph of Gene ontology (GO) analysis performed using top 300 up-regulated DEGs in hiNSCs compared with EPCs, showing count (26-38) over gene ratio (0.09 to 0.13) for each of the indicated biological functions. FIG. 5D is a heat map-representation of the Mean expression of selected NSC, PSC and EPC lineage markers, represented as log2 transformed read counts, for each of human induced pluripotent stem cells (hiPSC), H1-ESC, erythroid progenitor cells (EPC), H1-ESC-NPC, hiNSC-014HC1, hiNSC-014HC2, hiPSC-NPC, and hiNSC-C275 samples, respectively. FIG. 5E is a heat map-representation of Mean expression of selected forebrain, midbrain, hindbrain, spinal cord, dorsal and ventral regional identity genes represented as log2 transformed read counts, respectively.

FIGS. 6A-6C are bar graphs showing eSOX17FNV and C-MYC are sufficient to reprogram human blood cells into iNSCs. FIG. 6A is a graph of Gene expression of M/eSOX17FNV reprogrammed 2F-hiNSCs detected by qRT-PCR, showing expression of each of NES, SOX1, SOX2, PAX6, FABP7, and HESS relative to H9-NSC for each of Neonatal human dermal fibroblasts (HDFn), EPCs, hiPSCs, H9-NSCs and EPC-hiNSC, respectively. FIG. 6B is a graph of Gene expression of M/eSOX17FNV reprogrammed 2F-hiNSCs detected by qRT-PCR, showing expression of each of CD71, and CD117 relative to EPC for each of Neonatal human dermal fibroblasts (HDFn), EPCs, hiPSCs, H9-NSCs and EPC-hiNSC, respectively. FIG. 6C is a graph of Gene expression of M/eSOX17FNV reprogrammed 2F-hiNSCs detected by qRT-PCR, showing expression of each of POU5F1, and NANOG relative to hiPSC for each of Neonatal human dermal fibroblasts (HDFn), EPCs, hiPSCs, H9-NSCs and EPC-hiNSC, respectively.

### DETAILED DESCRIPTION OF THE INVENTION

### I. Definitions

The term “totipotency” (or totipotent), as used herein refers the potential of a cell to differentiate into extraembryonic cells such as trophectoderm (precursor of the placenta) or extraembryonic endoderm (precursor of the yolk sac). In addition, totipotent cells can give raise to all the cells of the embryo proper reminiscent to pluripotent cells.

The term “pluripotency” (or pluripotent), as used herein refers the potential of a cell to differentiate into any of the three germ layers: endoderm (for example, interior stomach lining, gastrointestinal tract, the lungs), mesoderm (for example, muscle, bone, blood, urogenital), or ectoderm (for example, epidermal tissues and nervous system). The term “not pluripotent” means that the cell does not have the potential to differentiate into all of the three germ layers. A multipotent stem cell is less plastic and more differentiated, and can become one of several types of cells within a given organ. For example, multipotent blood stem cells can develop into red blood cell progenitors, white blood cells or platelet producing cells. Adult stem cells are multipotent stem cells. Adipose-derived stem cells are multipotent.

“Reprogramming” refers to the conversion of a one specific cell type to another.

For example, a human somatic cell that is not pluripotent can be reprogrammed into a pluripotent cell. Where the non-pluripotent cell is reprogrammed into a pluripotent cell by exogenously introducing one or more genes, the resulting cell is an induced pluripotent stem cell.

The term “induced pluripotent stem cells” refers to pluripotent cells derived from a donor cell that is not pluripotent, i.e., a multipotent or differentiated cell, by engineering somatic cells to express one or more markers of pluripotency including POU5F1/OCT4 (Gene ID; 5460) in combination with, but not restricted to, SOX2 (Gene ID; 6657), KLF4 (Gene ID; 9314), cMYC (Gene ID; 4609), NANOG (Gene ID; 79923), LIN28/LIN28A (Gene ID; 79727)). The expression can be induced for example by forced gene expression or using small molecules, small RNAs, non-integrating gene expression vectors, or proteins.

The term “induced extended pluripotent stem cell” or “iEPSC” refers to a pluripotent stem cell with an improved ability to generate extraembryonic lineages in vitro or in vivo, by exogenously introducing one or more genes (e.g., transcription factors SOX17 (including mutants of SOX17, OCT4, KLF4, and/or C-MYC) into a donor cell and cultured in defined medium composition.

The term “induced neural stem cells” or “iNSC” refers to a neural stem cell with an ability to generate neurons, astrocytes and oligodendrocytes in vitro and/or in vivo. Typically, iNSCs are produced according to a method of exogenously introducing one or more genes (e.g., transcription factors SOX17 (including mutants of SOX17, C-MYC, and/or KLF4) into a donor cell, and culturing the cell in a culture medium having a composition suitable for trans-differentiation into an iNSC.

As used herein “enhancing,” or “increasing” the efficiency of reprogramming means reducing total reprograming time, enabling the reprogramming of challenging cells (such as senescent cells), increasing the number of reprogrammed cells obtained from the same starting cell density cultured for the same length of time and/or improving the quality of reprogrammed cells, measured in terms of characteristics selected from the ability of the cells to differentiate into embryonic and extraembryonic tissues and the number of passages in culture, when compared to those using conventional SOX2 transcription factor.

The term “express” refers to the transcription of a polynucleotide or translation of a polypeptide in a cell, such that levels of the molecule are measurably higher in a cell that expresses the molecule than they are in a cell that does not express the molecule. Methods to measure the expression of a molecule are well known to those of ordinary skill in the art, and include without limitation, Northern blotting, RT-PCR, in situ hybridization, Western blotting, and immunostaining such as FACS.

The term “culturing . . . with” is intended to include incubating the component(s) and the cell/tissue together in vitro (e.g., adding the compound to cells in culture) and the step of “culturing . . . with” can be conducted in any suitable manner. For example, the cells may be treated in adherent culture, in suspension culture, or in 3D culture; the components can be added temporally substantially simultaneously (e.g., together in a cocktail) or sequentially (e.g., within 1 hour, 1 day or more from an addition of a first component). The cells can also be contacted with another agent such as a growth factor or other differentiation agent or environments to stabilize the cells, or to differentiate the cells further and include culturing the cells under conditions known in the art.

### II. Compositions

Compositions of reagents for generating induced expanded-potential stem cells (iEPSCs) are provided. Compositions of iEPSCs are also provided.

In some forms, the compositions include cell culture reagents. Engineered SOX17 (eSOX17) transcription factors that enhance and induce cellular de-differentiation are provided, including minimal eSOX17-derivatives.

Exemplary compositions of reagents include transcription factor cocktails including engineered SOX17 factor, OCT4, KLF4, and C-MYC, and vectors carrying one or more of these transcription factors suitable for delivery to donor somatic cells for generating induced expanded potential stem cells (iEPSCs). iEPSCs generated according to the cell culture systems, as well as differentiated cells derived therefrom are also provided. In some forms, the methods provide eSOX17-derived expanded potential stem cells (iEPSCs). In some forms, the methods provide eSOX17-derived multipotent induced neural stem cells (iNSCs). Evaluation of the determinants for improved pluripotency induction and maintenance by engineered SOX17 is also described in Hu, et al., Nucleic Acids Research, pp.1-23, 2023, the contents of which are incorporated by reference herein in their entirety.

A. Induced Expanded Potential Stem Cells (iEPSCs)

Compositions of Induced Expanded Potential Stem Cells (iEPSCs) are described. In has been established that the forcible expression of defined transcription factor (TF) cocktails can effectively engage and reprogram the epigenome of somatic cells, leading to drastic cell-fate conversions. As demonstrated in the Examples, improved human induced expanded potential stem cells (iEPSCs) have been generated from engineered SOX17 factor along with OCT4, KLF4, and C-MYC.

Typically, the iEPSCs are distinguished from naturally-occurring non-differentiated cells. In some forms, the eSOX17-derived iEPSCs are Sox2+ cells generated from embryonic, adult or aged “parent” cells. In some forms, the eSOX17-derived iEPSCs do not express one or more genes that are typically expressed by the somatic “parent” cell from which they are derived.

Contrary to naturally occurring pluripotent cells that can only be derived from human embryos, iEPSCs do not require the destruction of human embryos as they can be generated from somatic human cells such as skin biopsies, blood or urine. Typically, the described eSOX17-derived iEPSCs are molecularly and functionally equivalent to their endogenous counterparts.

In some forms, the iEPSCs are human iEPSCs. Therefore, the iEPSCs are derived from one or more human cells. In some forms, the iEPSCs are formulated into a pharmaceutical formulation for administration to a subject in vivo. In other forms, when the iEPSCs are formulated into a formulation for culturing and/or maintaining the cells in vitro. In some forms, formulations of iEPSCs include exclusively iEPSCs. Therefore, in some forms, the only viable cells in formulations of iEPSCs are totipotent and/or multipotent cells derived from differentiated cells according to the described methods. In other forms, formulations of iEPSCs include one or more viable cells that are not totipotent and/or multipotent cells derived from differentiated cells according to the described methods. Therefore, in some forms, the amount of totipotent and/or multipotent cells derived from differentiated cells according to the described methods present in formulations of iEPSCs is less than 100% of the total number of viable cells in the formulation, such as 99%, 98%, 97%, 96%, 95%, 94%, 93%, 92%, 91%, 90%, or less than 90%, such as e.g., at least 85%; at least 80%; at least 75%; at least 70%; at least 60%; at least 50%, or less than 50%, such as 40%, 30%, 20%, 10%, 5%, 1% , or less than 1% of the total number of viable cells in the formulation. In some forms, when the iEPSCs are formulated into a pharmaceutical formulation for administration to a subject in vivo, the iEPSCs are autologous, i.e., the iEPSCs are derived from cells obtained from the same subject that is the recipient of the iEPSCs. In other forms, when the iEPSCs are formulated into a pharmaceutical formulation for administration to a subject in vivo, the iEPSCs are not autologous, i.e., the iEPSCs are derived from cells obtained from a different subject from the recipient of the iEPSCs. In some forms, formulations of iEPSCs for administration to a subject in vivo include a mixture of autologous an non-autologous iEPSCs.

In some forms, the iEPSCs are derived from engineered transcription factors, such as engineered SOX17 (eSOX17), for example, according to the methods described herein. Therefore, eSOX17-derived iEPSCs are provided. Typically, the described eSOX17-derived iEPSCs have a lower tumorigenic risk in a differentiated cell population as compared to that of induced pluripotent stem cells (iPSCs) reprogrammed via other means, such as the overexpression of Oct4, Sox2, Klf4 and c-Myc (OSKM).

B. Induced Neural Stem Cells (iNSC)

Induced Neural Stem Cells (iNSC) generated with transcription factors, such as engineered SOX17 (eSOX17) are described. Exemplary iNSCs are multipotent iNSCs, such as tripotent iNSCs. For example, in some forms, iNSCs are derived by trans-differentiation of somatic cells, such as blood cells. The iNSCs directly generated with eSOX17 factors from aged human blood cells show the potential to avoid full epigenetic rejuvenation. This designates iNSCs as a more suitable cell source than pluripotent stem cells (PSCs) to model aging-related neurodegenerative diseases.

In some forms, eSOX17-derived iNSCs are not pluripotent. The three major cell types in the CNS arise from NSCs in a temporally defined sequence, with neurons appearing first, followed by astrocytes, and then oligodendrocytes. Therefore, in some forms, the described eSOX17-derived iNSCs are tri-potent iNSCs, i.e., having the ability to differentiate into neurons, astrocytes, and oligodendrocytes in vitro and/or in vivo, for example, upon exposure to the appropriate culture conditions/media.

Typically, eSOX17-derived iNSCs exhibit different gene expression analysis as compared with parent somatic cells from which they are derived, and also as compared with pluripotent stem cells (PSC). In some forms, eSOX17-derived iNSCs exhibit an enrichment for neuronal functions such as synapse organization, axonogenesis, and regulation of neurogenesis. In some forms, the eSOX17-derived iNSCs express the NSC markers NESTIN, SOX1, SOX2, PAX6, and FABP7 at protein and transcript levels. In some forms, the eSOX17-derived iNSCs do not express one or more genes that are typically expressed by the somatic “parent” cell from which they are derived. In some forms, the eSOX17-derived iNSCs do not express one or more genes associated with pluripotency. In some forms, the eSOX17-derived iNSCs do not express one POU5F1 and/or NANOG.

The iNSCs directly generated with eSOX17 factors from aged human blood cells show the potential to avoid full epigenetic rejuvenation. Therefore, in some forms, iNSCs produced according to the described methods provide a more suitable cell source than pluripotent stem cells (PSCs) to model aging-related neurodegenerative diseases. Lineage tracing and time-resolved transcriptomics show that emerging iNSCs do not transit through a pluripotent state.

Unlike iPSC-derived neurons, induced neurons (iNs) are generally barely rejuvenated and closely reflect the transcriptional and epigenetic signatures of aging. The described eSOX17-derived iNSCs are tripotent and are readily produced at scale or used as starting materials for organoids. Thus, in some forms, eSOX17-derived iNSCs are models for aging-related neurodegenerative diseases.

Neural stem cells naturally occur in the central nervous system such as human brains or spinal cords. It is practically impossible to obtain these cells from live individuals via surgical procedures. As described herein, eSOX17-derived iNSCs can be directly generated from somatic human blood, skin or urine cells. Typically, the described eSOX17-derived iNSCs are molecularly and functionally equivalent to their endogenous counterparts.

### C. Reagents for Inducing Totipotency/Multipotency

Compositions of reagents for generating, and/or maintaining Induced Expanded Potential Stem Cells (iEPSCs) are described. Typically, the reagents include engineered transcription factors. An exemplary engineered transcription factor includes SOX17 (eSOX17). Typically, engineered transcription factors, such as eSOX17 are administered into target cells within a suitable vector for expression of the engineered transcription factor within a target cell. Therefore, suitable vectors encoding and/or expressing the engineered transcription factors, for delivery and/or expression of engineered transcription factors into cells are also provided.

1. Engineered SOX17 (eSOX17)

Engineered SOX17 transcription factors are provided. An earlier study using an approach termed directed evolution of reprogramming factors by cell selection and sequencing (DERBY-seq) identified engineered transcription factors (Veerapandian V, et al., Stem Cell Reports. 2018;11(2):593-606).

It has been established that engineered SOX17 (eSOX17) factors can substantially enhance the reprogramming efficiency of human induced expanded potential stem cells (iEPSCs) which represent a state reminiscent to totipotent cells. eSox17FNV efficiently drives iNSC reprogramming while Sox2 or Sox17 fail. eSox17FNV acquires the capacity to bind different protein partners on regulatory DNA to scan the genome more efficiently and possesses a more potent transactivation domain than Sox2. Therefore, in some forms, eSox17FNV functions to change protein partners and scans chromatin to a greater extent as compared to wild-type Sox2 and/or wild-type Sox17.

It has also been established that eSOX17 functions to transdifferentiate somatic cells, such as human blood cells, into multipotent induced neural stem cells (INSCs) without detour to pluripotency.

Compared to conventionally used SOX2, eSOX17 converts cells more rapidly and efficiently and even reprograms recalcitrant cells such as high passage senescent fibroblasts where SOX2 fails. These eSOX17-derived induced iEPSCs are more likely to form blastoids suggesting more complete reprogramming and more pronounced totipotency features.

Built upon the concept of directed biomolecular evolution in protein engineering to tailor and enhance enzymes and antibodies, several candidates were identified and validated based on high throughput screen of SOX17 protein libraries in mouse fibroblast reprogramming.

In some forms, eSOX17 has an increased preference for binding to the canonical Sox2:Oct4 motif in chromatin rather than the compressed Sox2:Oct4 motif in chromatin. In some forms, eSOX17 has an increased preference for binding to the canonical Sox2:Oct4 motif in chromatin as compared to wild type SOX17. Therefore, in some forms, eSOX17 includes one or more mutations that enhance preference for binding to the canonical Sox2:Oct4 motif in chromatin as compared to wild type SOX17.

In some forms, eSOX17 cooperatively dimerizes with other factors more tightly and/or with greater affinity as compared with wild type SOX17. For example, in some forms, eSOX17 cooperatively dimerizes with the neural POU factor Brn2 on the canonical SoxOct motif to a greater extent than the corresponding dimerization of the neural POU factor Brn2 with wild type SOX17. Therefore, in some forms, eSOX17 includes one or more mutations that enhance the dimerization with the neural POU factor Brn2 relative to wild type SOX17.

In some forms, eSOX17 includes mutation of one or more amino acids in the DNA-binding domain (DBD) that affects protein partnerships and co-binding to DNA that profoundly change diffusion, chromatin scanning and/or dwell time as compared with wild type SOX17. For example, in some forms, eSOX17 includes mutation of one or more amino acids in the DNA-binding domain (DBD) that enhances the efficiency to search for target genes as compared with wild type SOX17.

In some forms, eSOX17 includes mutation of one or more amino acids in the C-terminal transactivation domain (TAD) that enhances one or more functions of eSOX17, as compared with wild type SOX17.

In preferred forms, the engineered SOX17 factor includes the wild-type SOX17 polypeptide, including triple amino acid substitutions, i.e., at positions L111F, V118N, and E122V (termed “SOX17FNV”). As set forth in the Examples, B/K/M/eSox17FNV efficiently generated Sox2-GFP+ colonies, whereas no Sox2-GFP+ colonies were generated with the neural stem cell factor Sox2 or the endodermal gene Sox17. In an exemplary form, the engineered SOX17FNV factor has the following amino acid sequence:
MSSPDAGYASDDQSQTQSALPAVMAGLGPCPWAESLSPIGDMKVKGEAPANSGA PAGAAGRAKGESRIRRPMNAFMVWAKDERKRLAQQNPDLHNAELSKMLGKSWKALTFAE KRPFNEEAVRLRVQHMQDHPNYKYRPRRRKQVKRLKRVEGGFLHGLAEPQAAALGPEGG RVAMDGLGLQFPEQGFPAGPPLLPPHMGGHYRDCQSLGAPPLDGYPLPTPDTSPLDGVD PDPAFFAAPMPGDCPAAGTYSYAQVSDYAGPPEPPAGPMHPRLGPEPAGPSIPGLLAPP SALHVYYGAMGSPGAGGGRGFQMQPQHQHQHQHQHHPPGPGQPSPPPEALPCRDGTDPS QPAELLGEVDRTEFEQYLHFVCKPEMGLPYQGHDSGVNLPDSHGAISSVVSDASSAVYY CNYPDV* (SOX17FNV; SEQ ID NO:1), or an amino acid sequence having at least 70%, at least 75%, at least 80%, at least 85%, at least 90%, at least 95%, at least 96%, at least 20 97%, at least 98%, at least 99%, or 100% sequence identify with SOX17FNV of SEQ ID NO:1, or a fragment thereof.

It has been established that the amino acid at position 57 (bolded and italicized) of the HMG box of the mature protein has to be mutated to convert SOX17 into an inducer of pluripotency and to generate eSOX17 variants. Accordingly, eSOX17 transcription factors are defined as artificial factors that have a high sequence similarity to wild-type SOX17, but which contain mutations of the amino acid at position 57 (of the HMG box. Mutations of position 53 to N and position 46 to F are also indicated in bold text.

An exemplary HMG polypeptide sequence of SOX17FNV includes:

(SEQ ID NO: 4)
SRIRRPMNAFMVWAKDERKRLAQQNPDLHNAELSKMLGKS
WKALTFAEKRPFNEEAVRLRVQHMQDHPNYKYRPRRRKQ.

Engineered SOX17 polypeptides include a mutation of the amino acid at position 57 of the wild-type SOX17 protein. Typically, eSOX17 polypeptides include substitution of the naturally occurring glutamate residue, for example, with a valine (V).

SOX17FNV is one example of an eSOX17. SOX17FNV contains an E57V mutation. However, eSOX17 variants having any other mutations at this position are also contemplated. SOX17FNV contains additional accessory mutations at position 46 and 53 of the HMG box. In some forms, the engineered SOX17 polypeptide is a variant of SOX17 including a non-acidic amino acid such as alanine (A), arginine (R), asparagine (N), cysteine (C), glutamine (Q), glycine (G), histidine (H), isoleucine (I), leucine (L), lysine (K), methionine (M), phenylalanine (F), serine (S), threonine (T), tryptophan (W), and tyrosine (Y), instead of the naturally occurring glutamate residue at position 57 of the HMG box of the wild-type SOX17 protein. In some forms, the eSOX17 is a variant having an amino acid sequence having at least 95% sequence identity to SEQ ID NO: 1. In some forms, a derivative of eSOX17, named as minimal SOX, is a variant with full deletion of the N-terminus amino acids and partial deletion of the C-terminus amino acids of eSOX17, which confer similar degree of pluripotency as full-length engineered SOX17 factors, such as SOX17FNV. Exemplary variants of eSOX17are indicated below, with each mutation at position 57 indicated in bold, italic text. The variant eSOX17 sequences include:

MSSPDAGYASDDQSQTQSALPAVMAGLGPCPWAESLSPIGDMKVKGEAPANSGA
(SOX17E57A; SEQ ID NO: 5)
PAGAAGRAKGESRIRRPMNAFMVWAKDERKRLAQQNPDLHNAELSKMLGKSWKALTLAE
KRPFVEEAARLRVQHMQDHPNYKYRPRRRKQVKRLKRVEGGFLHGLAEPQAAALGPEGG
RVAMDGLGLQFPEQGFPAGPPLLPPHMGGHYRDCQSLGAPPLDGYPLPTPDTSPLDGVD
PDPAFFAAPMPGDCPAAGTYSYAQVSDYAGPPEPPAGPMHPRLGPEPAGPSIPGLLAPP
SALHVYYGAMGSPGAGGGRGFQMQPQHQHQHQHQHHPPGPGQPSPPPEALPCRDGTDPS
QPAELLGEVDRTEFEQYLHFVCKPEMGLPYQGHDSGVNLPDSHGAISSVVSDASSAVYY
CNYPDV;
(SOX17E57R; SEQ ID NO: 6)
MSSPDAGYASDDQSQTQSALPAVMAGLGPCPWAESLSPIGDMKVKGEAPANSGA
PAGAAGRAKGESRIRRPMNAFMVWAKDERKRLAQQNPDLHNAELSKMLGKSWKALTLAE
KRPFVEEARRLRVQHMQDHPNYKYRPRRRKQVKRLKRVEGGFLHGLAEPQAAALGPEGG
RVAMDGLGLQFPEQGFPAGPPLLPPHMGGHYRDCQSLGAPPLDGYPLPTPDTSPLDGVD
PDPAFFAAPMPGDCPAAGTYSYAQVSDYAGPPEPPAGPMHPRLGPEPAGPSIPGLLAPP
SALHVYYGAMGSPGAGGGRGFQMQPQHQHQHQHQHHPPGPGQPSPPPEALPCRDGTDPS
QPAELLGEVDRTEFEQYLHFVCKPEMGLPYQGHDSGVNLPDSHGAISSVVSDASSAVYY
CNYPDV;
(SOX17E57N; SEQ ID NO: 7)
MSSPDAGYASDDQSQTQSALPAVMAGLGPCPWAESLSPIGDMKVKGEAPANSGA
PAGAAGRAKGESRIRRPMNAFMVWAKDERKRLAQQNPDLHNAELSKMLGKSWKALTLAE
KRPFVEEANRLRVQHMQDHPNYKYRPRRRKQVKRLKRVEGGFLHGLAEPQAAALGPEGG
RVAMDGLGLQFPEQGFPAGPPLLPPHMGGHYRDCQSLGAPPLDGYPLPTPDTSPLDGVD
PDPAFFAAPMPGDCPAAGTYSYAQVSDYAGPPEPPAGPMHPRLGPEPAGPSIPGLLAPP
SALHVYYGAMGSPGAGGGRGFQMQPQHQHQHQHQHHPPGPGQPSPPPEALPCRDGTDPS
QPAELLGEVDRTEFEQYLHFVCKPEMGLPYQGHDSGVNLPDSHGAISSVVSDASSAVYY
CNYPDV;
(SOX17E57C; SEQ ID NO: 8)
MSSPDAGYASDDQSQTQSALPAVMAGLGPCPWAESLSPIGDMKVKGEAPANSGA
PAGAAGRAKGESRIRRPMNAFMVWAKDERKRLAQQNPDLHNAELSKMLGKSWKALTLAE
KRPFVEEACRLRVQHMQDHPNYKYRPRRRKQVKRLKRVEGGFLHGLAEPQAAALGPEGG
RVAMDGLGLQFPEQGFPAGPPLLPPHMGGHYRDCQSLGAPPLDGYPLPTPDTSPLDGVD
PDPAFFAAPMPGDCPAAGTYSYAQVSDYAGPPEPPAGPMHPRLGPEPAGPSIPGLLAPP
SALHVYYGAMGSPGAGGGRGFQMQPQHQHQHQHQHHPPGPGQPSPPPEALPCRDGTDPS
QPAELLGEVDRTEFEQYLHFVCKPEMGLPYQGHDSGVNLPDSHGAISSVVSDASSAVYY
CNYPDV;
(SOX17E57Q; SEQ ID NO: 9)
MSSPDAGYASDDQSQTQSALPAVMAGLGPCPWAESLSPIGDMKVKGEAPANSGA
PAGAAGRAKGESRIRRPMNAFMVWAKDERKRLAQQNPDLHNAELSKMLGKSWKALTLAE
KRPFVEEAQRLRVQHMQDHPNYKYRPRRRKQVKRLKRVEGGFLHGLAEPQAAALGPEGG
RVAMDGLGLQFPEQGFPAGPPLLPPHMGGHYRDCQSLGAPPLDGYPLPTPDTSPLDGVD
PDPAFFAAPMPGDCPAAGTYSYAQVSDYAGPPEPPAGPMHPRLGPEPAGPSIPGLLAPP
SALHVYYGAMGSPGAGGGRGFQMQPQHQHQHQHQHHPPGPGQPSPPPEALPCRDGTDPS
QPAELLGEVDRTEFEQYLHFVCKPEMGLPYQGHDSGVNLPDSHGAISSVVSDASSAVYY
CNYPDV;
(SOX17E57G; SEQ ID NO: 10)
MSSPDAGYASDDQSQTQSALPAVMAGLGPCPWAESLSPIGDMKVKGEAPANSGA
PAGAAGRAKGESRIRRPMNAFMVWAKDERKRLAQQNPDLHNAELSKMLGKSWKALTLAE
KRPFVEEAGRLRVQHMQDHPNYKYRPRRRKQVKRLKRVEGGFLHGLAEPQAAALGPEGG
RVAMDGLGLQFPEQGFPAGPPLLPPHMGGHYRDCQSLGAPPLDGYPLPTPDTSPLDGVD
PDPAFFAAPMPGDCPAAGTYSYAQVSDYAGPPEPPAGPMHPRLGPEPAGPSIPGLLAPP
SALHVYYGAMGSPGAGGGRGFQMQPQHQHQHQHQHHPPGPGQPSPPPEALPCRDGTDPS
QPAELLGEVDRTEFEQYLHFVCKPEMGLPYQGHDSGVNLPDSHGAISSVVSDASSAVYY
CNYPDV;
(SOX17E57H; SEQ ID NO: 11)
MSSPDAGYASDDQSQTQSALPAVMAGLGPCPWAESLSPIGDMKVKGEAPANSGA
PAGAAGRAKGESRIRRPMNAFMVWAKDERKRLAQQNPDLHNAELSKMLGKSWKALTLAE
KRPFVEEAHRLRVQHMQDHPNYKYRPRRRKQVKRLKRVEGGFLHGLAEPQAAALGPEGG
RVAMDGLGLQFPEQGFPAGPPLLPPHMGGHYRDCQSLGAPPLDGYPLPTPDTSPLDGVD
PDPAFFAAPMPGDCPAAGTYSYAQVSDYAGPPEPPAGPMHPRLGPEPAGPSIPGLLAPP
SALHVYYGAMGSPGAGGGRGFQMQPQHQHQHQHQHHPPGPGQPSPPPEALPCRDGTDPS
QPAELLGEVDRTEFEQYLHFVCKPEMGLPYQGHDSGVNLPDSHGAISSVVSDASSAVYY
CNYPDV;
(SOX17E57I; SEQ ID NO: 12)
MSSPDAGYASDDQSQTQSALPAVMAGLGPCPWAESLSPIGDMKVKGEAPANSGA
PAGAAGRAKGESRIRRPMNAFMVWAKDERKRLAQQNPDLHNAELSKMLGKSWKALTLAE
KRPFVEEAIRLRVQHMQDHPNYKYRPRRRKQVKRLKRVEGGFLHGLAEPQAAALGPEGG
RVAMDGLGLQFPEQGFPAGPPLLPPHMGGHYRDCQSLGAPPLDGYPLPTPDTSPLDGVD
PDPAFFAAPMPGDCPAAGTYSYAQVSDYAGPPEPPAGPMHPRLGPEPAGPSIPGLLAPP
SALHVYYGAMGSPGAGGGRGFQMQPQHQHQHQHQHHPPGPGQPSPPPEALPCRDGTDPS
QPAELLGEVDRTEFEQYLHFVCKPEMGLPYQGHDSGVNLPDSHGAISSVVSDASSAVYY
CNYPDV;
(SOX17E57L; SEQ ID NO: 13)
MSSPDAGYASDDQSQTQSALPAVMAGLGPCPWAESLSPIGDMKVKGEAPANSGA
PAGAAGRAKGESRIRRPMNAFMVWAKDERKRLAQQNPDLHNAELSKMLGKSWKALTLAE
KRPFVEEALRLRVQHMQDHPNYKYRPRRRKQVKRLKRVEGGFLHGLAEPQAAALGPEGG
RVAMDGLGLQFPEQGFPAGPPLLPPHMGGHYRDCQSLGAPPLDGYPLPTPDTSPLDGVD
PDPAFFAAPMPGDCPAAGTYSYAQVSDYAGPPEPPAGPMHPRLGPEPAGPSIPGLLAPP
SALHVYYGAMGSPGAGGGRGFQMQPQHQHQHQHQHHPPGPGQPSPPPEALPCRDGTDPS
QPAELLGEVDRTEFEQYLHFVCKPEMGLPYQGHDSGVNLPDSHGAISSVVSDASSAVYY
CNYPDV;
(SOX17E57K; SEQ ID NO: 14)
MSSPDAGYASDDQSQTQSALPAVMAGLGPCPWAESLSPIGDMKVKGEAPANSGA
PAGAAGRAKGESRIRRPMNAFMVWAKDERKRLAQQNPDLHNAELSKMLGKSWKALTLAE
KRPFVEEAKRLRVQHMQDHPNYKYRPRRRKQVKRLKRVEGGFLHGLAEPQAAALGPEGG
RVAMDGLGLQFPEQGFPAGPPLLPPHMGGHYRDCQSLGAPPLDGYPLPTPDTSPLDGVD
PDPAFFAAPMPGDCPAAGTYSYAQVSDYAGPPEPPAGPMHPRLGPEPAGPSIPGLLAPP
SALHVYYGAMGSPGAGGGRGFQMQPQHQHQHQHQHHPPGPGQPSPPPEALPCRDGTDPS
QPAELLGEVDRTEFEQYLHFVCKPEMGLPYQGHDSGVNLPDSHGAISSVVSDASSAVYY
CNYPDV;
(SOX17E57M; SEQ ID NO: 15)
MSSPDAGYASDDQSQTQSALPAVMAGLGPCPWAESLSPIGDMKVKGEAPANSGA
PAGAAGRAKGESRIRRPMNAFMVWAKDERKRLAQQNPDLHNAELSKMLGKSWKALTLAE
KRPFVEEAMRLRVQHMQDHPNYKYRPRRRKQVKRLKRVEGGFLHGLAEPQAAALGPEGG
RVAMDGLGLQFPEQGFPAGPPLLPPHMGGHYRDCQSLGAPPLDGYPLPTPDTSPLDGVD
PDPAFFAAPMPGDCPAAGTYSYAQVSDYAGPPEPPAGPMHPRLGPEPAGPSIPGLLAPP
SALHVYYGAMGSPGAGGGRGFQMQPQHQHQHQHQHHPPGPGQPSPPPEALPCRDGTDPS
QPAELLGEVDRTEFEQYLHFVCKPEMGLPYQGHDSGVNLPDSHGAISSVVSDASSAVYY
CNYPDV;
(SOX17E57F; SEQ ID NO: 16)
MSSPDAGYASDDQSQTQSALPAVMAGLGPCPWAESLSPIGDMKVKGEAPANSGA
PAGAAGRAKGESRIRRPMNAFMVWAKDERKRLAQQNPDLHNAELSKMLGKSWKALTLAE
KRPFVEEAFRLRVQHMQDHPNYKYRPRRRKQVKRLKRVEGGFLHGLAEPQAAALGPEGG
RVAMDGLGLQFPEQGFPAGPPLLPPHMGGHYRDCQSLGAPPLDGYPLPTPDTSPLDGVD
PDPAFFAAPMPGDCPAAGTYSYAQVSDYAGPPEPPAGPMHPRLGPEPAGPSIPGLLAPP
SALHVYYGAMGSPGAGGGRGFQMQPQHQHQHQHQHHPPGPGQPSPPPEALPCRDGTDPS
QPAELLGEVDRTEFEQYLHFVCKPEMGLPYQGHDSGVNLPDSHGAISSVVSDASSAVYY
CNYPDV;
(SOX17E57S; SEQ ID NO: 17)
MSSPDAGYASDDQSQTQSALPAVMAGLGPCPWAESLSPIGDMKVKGEAPANSGA
PAGAAGRAKGESRIRRPMNAFMVWAKDERKRLAQQNPDLHNAELSKMLGKSWKALTLAE
KRPFVEEASRLRVQHMQDHPNYKYRPRRRKQVKRLKRVEGGFLHGLAEPQAAALGPEGG
RVAMDGLGLQFPEQGFPAGPPLLPPHMGGHYRDCQSLGAPPLDGYPLPTPDTSPLDGVD
PDPAFFAAPMPGDCPAAGTYSYAQVSDYAGPPEPPAGPMHPRLGPEPAGPSIPGLLAPP
SALHVYYGAMGSPGAGGGRGFQMQPQHQHQHQHQHHPPGPGQPSPPPEALPCRDGTDPS
QPAELLGEVDRTEFEQYLHFVCKPEMGLPYQGHDSGVNLPDSHGAISSVVSDASSAVYY
CNYPDV;
(SOX17E57T; SEQ ID NO: 18)
MSSPDAGYASDDQSQTQSALPAVMAGLGPCPWAESLSPIGDMKVKGEAPANSGA
PAGAAGRAKGESRIRRPMNAFMVWAKDERKRLAQQNPDLHNAELSKMLGKSWKALTLAE
KRPFVEEATRLRVQHMQDHPNYKYRPRRRKQVKRLKRVEGGFLHGLAEPQAAALGPEGG
RVAMDGLGLQFPEQGFPAGPPLLPPHMGGHYRDCQSLGAPPLDGYPLPTPDTSPLDGVD
PDPAFFAAPMPGDCPAAGTYSYAQVSDYAGPPEPPAGPMHPRLGPEPAGPSIPGLLAPP
SALHVYYGAMGSPGAGGGRGFQMQPQHQHQHQHQHHPPGPGQPSPPPEALPCRDGTDPS
QPAELLGEVDRTEFEQYLHFVCKPEMGLPYQGHDSGVNLPDSHGAISSVVSDASSAVYY
CNYPDV;
(SOX17E57W; SEQ ID NO: 19)
MSSPDAGYASDDQSQTQSALPAVMAGLGPCPWAESLSPIGDMKVKGEAPANSGA
PAGAAGRAKGESRIRRPMNAFMVWAKDERKRLAQQNPDLHNAELSKMLGKSWKALTLAE
KRPFVEEAWRLRVQHMQDHPNYKYRPRRRKQVKRLKRVEGGFLHGLAEPQAAALGPEGG
RVAMDGLGLQFPEQGFPAGPPLLPPHMGGHYRDCQSLGAPPLDGYPLPTPDTSPLDGVD
PDPAFFAAPMPGDCPAAGTYSYAQVSDYAGPPEPPAGPMHPRLGPEPAGPSIPGLLAPP
SALHVYYGAMGSPGAGGGRGFQMQPQHQHQHQHQHHPPGPGQPSPPPEALPCRDGTDPS
QPAELLGEVDRTEFEQYLHFVCKPEMGLPYQGHDSGVNLPDSHGAISSVVSDASSAVYY
CNYPDV;
and
(SOX17E57Y; SEQ ID NO: 20)
MSSPDAGYASDDQSQTQSALPAVMAGLGPCPWAESLSPIGDMKVKGEAPANSGA
PAGAAGRAKGESRIRRPMNAFMVWAKDERKRLAQQNPDLHNAELSKMLGKSWKALTLAE
KRPFVEEAYRLRVQHMQDHPNYKYRPRRRKQVKRLKRVEGGFLHGLAEPQAAALGPEGG
RVAMDGLGLQFPEQGFPAGPPLLPPHMGGHYRDCQSLGAPPLDGYPLPTPDTSPLDGVD
PDPAFFAAPMPGDCPAAGTYSYAQVSDYAGPPEPPAGPMHPRLGPEPAGPSIPGLLAPP
SALHVYYGAMGSPGAGGGRGFQMQPQHQHQHQHQHHPPGPGQPSPPPEALPCRDGTDPS
QPAELLGEVDRTEFEQYLHFVCKPEMGLPYQGHDSGVNLPDSHGAISSVVSDASSAVYY
CNYPDV.

In some forms, the eSOX17 is a variant having an amino acid sequence having at least 95% sequence identity to any one of SEQ ID NOs:5-20.

In some forms, the eSOX17 is a variant having an HMG amino acid sequence having at least 95% sequence identity to SEQ ID NO:4. In some forms, the engineered SOX17 HMG polypeptide is a variant of the HMG polypeptide of SOX17 including a non-acidic amino acid such as alanine (A), arginine (R), asparagine (N), cysteine (C), glutamine (Q), glycine (G), histidine (H), isoleucine (I), leucine (L), lysine (K), methionine (M), phenylalanine (F), serine (S), threonine (T), tryptophan (W), and tyrosine (Y), instead of the naturally occurring glutamate residue at position 57 of the HMG box of the wild-type SOX17 protein. Therefore, eSOX17 variant proteins having an HMG amino acid sequence including any of these substitutions are provided.

An exemplary HMG polypeptide sequence of SOX17E57A includes:

(SEQ ID NO: 21)
SRIRRPMNAFMVWAKDERKRLAQQNPDLHNAELSKMLGKS
WKALTLAEKRPFVEEAARLRVQHMQDHPNYKYRPRRRKQ;

An exemplary HMG polypeptide sequence of SOX17E57R includes:

(SEQ ID NO: 22)
SRIRRPMNAFMVWAKDERKRLAQQNPDLHNAELSKMLGKS
WKALTLAEKRPFVEEARRLRVQHMQDHPNYKYRPRRRKQ;

An exemplary HMG polypeptide sequence of SOX17E57N includes:

(SEQ ID NO: 23)
SRIRRPMNAFMVWAKDERKRLAQQNPDLHNAELSKMLGKSWKALTLAEKRP
FVEEANRLRVQHMQDHPNYKYRPRRRKQ;

An exemplary HMG polypeptide sequence of SOX17E57C includes:

(SEQ ID NO: 24)
SRIRRPMNAFMVWAKDERKRLAQQNPDLHNAELSKMLGKSWKALTLAEKRP
FVEEACRLRVQHMQDHPNYKYRPRRRKQ;

An exemplary HMG polypeptide sequence of SOX17E57Q includes:

(SEQ ID NO: 25)
SRIRRPMNAFMVWAKDERKRLAQQNPDLHNAELSKMLGKSWKALTLAEKRP
FVEEAQRLRVQHMQDHPNYKYRPRRRKQ;

An exemplary HMG polypeptide sequence of SOX17E57G includes:

(SEQ ID NO: 26)
SRIRRPMNAFMVWAKDERKRLAQQNPDLHNAELSKMLGKSWKALTLAEKRP
FVEEAGRLRVQHMQDHPNYKYRPRRRKQ;

An exemplary HMG polypeptide sequence of SOX17E57H includes:

(SEQ ID NO: 27)
SRIRRPMNAFMVWAKDERKRLAQQNPDLHNAELSKMLGKSWKALTLAEKRP
FVEEAHRLRVQHMQDHPNYKYRPRRRKQ;

An exemplary HMG polypeptide sequence of SOX17E57I includes:

(SEQ ID NO: 28)
SRIRRPMNAFMVWAKDERKRLAQQNPDLHNAELSKMLGKSWKALTLAEKRP
FVEEAIRLRVQHMQDHPNYKYRPRRRKQ;

An exemplary HMG polypeptide sequence of SOX17E57L includes:

(SEQ ID NO: 29)
SRIRRPMNAFMVWAKDERKRLAQQNPDLHNAELSKMLGKSWKALTLAEKRP
FVEEALRLRVQHMQDHPNYKYRPRRRKQ;

An exemplary HMG polypeptide sequence of SOX17E57K includes:

(SEQ ID NO: 30)
SRIRRPMNAFMVWAKDERKRLAQQNPDLHNAELSKMLGKSWKALTLAEKRP
FVEEAKRLRVQHMQDHPNYKYRPRRRKQ;

An exemplary HMG polypeptide sequence of SOX17E57M includes:

(SEQ ID NO: 31)
SRIRRPMNAFMVWAKDERKRLAQQNPDLHNAELSKMLGKSWKALTLAEKRP
FVEEAMRLRVQHMQDHPNYKYRPRRRKQ;

An exemplary HMG polypeptide sequence of SOX17E57F includes:

(SEQ ID NO: 32)
SRIRRPMNAFMVWAKDERKRLAQQNPDLHNAELSKMLGKSWKALTLAEKRP
FVEEAFRLRVQHMQDHPNYKYRPRRRKQ;

An exemplary HMG polypeptide sequence of SOX17E57S includes:

(SEQ ID NO: 33)
SRIRRPMNAFMVWAKDERKRLAQQNPDLHNAELSKMLGKSWKALTLAEKRP
FVEEASRLRVQHMQDHPNYKYRPRRRKQ;

An exemplary HMG polypeptide sequence of SOX17E57T includes:

(SEQ ID NO: 34)
SRIRRPMNAFMVWAKDERKRLAQQNPDLHNAELSKMLGKSWKALTLAEKRP
FVEEATRLRVQHMQDHPNYKYRPRRRKQ;

An exemplary HMG polypeptide sequence of SOX17E57W includes:

(SEQ ID NO: 35)
SRIRRPMNAFMVWAKDERKRLAQQNPDLHNAELSKMLGKSWKALTLAEKRP
FVEEAWRLRVQHMQDHPNYKYRPRRRKQ;

An exemplary HMG polypeptide sequence of SOX17E57Y includes:

(SEQ ID NO: 36)
SRIRRPMNAFMVWAKDERKRLAQQNPDLHNAELSKMLGKSWKALTLAEKR
PFVEEAYRLRVQHMQDHPNYKYRPRRRKQ;

and

An exemplary HMG polypeptide sequence of SOX17E57V includes:

(SEQ ID NO: 37)
SRIRRPMNAFMVWAKDERKRLAQQNPDLHNAELSKMLGKS
WKALTLAEKRPFVEEAVRLRVQHMQDHPNYKYRPRRRKQ.

The engineered SOX17 factor(s) enhance the reprogramming efficiency of EPSCs from somatic cells (e.g., up to 5% efficiency in fibroblast reprogramming), and show promising reprogramming capacity to senescent fibroblasts. These engineered SOX17 factors (eSOX17) outperform wild type SOX2 in pluripotency reprogramming as shown below in Examples in terms of speed and efficiency. Thus, in some forms, the engineered SOX17 factor in combination with OCT4, KLF4, and C-MYC can provide more than 100% reprogrammed EPSCs compared to the equivalent set-up but using a wild type SOX2 instead of the engineered SOX17 factor.

These eSOX17-derived iEPSCs are more likely to form blastoids suggesting their pronounced totipotency features. eSOX17 derived iEPSCs are considered as the ideal cell model in preclinical study of infertility-related diseases and basic research of early human development to avoid the ethical problems specific to the use of human embryo and blastocysts. Further, the robust and efficient eSOX17-driven iEPSC reprogramming provide opportunities to generate personalized models for reproductive disorders using patient-derived cells.

i. Minimal eSOX17 Protein

In some forms, the engineered SOX17 transcription factor is a minimal SOX transcription factor (miniSOX). Generally, the minimal, shortened version of eSOX has up to 30% less protein mass/polypeptide length than eSOX17. Therefore, in some forms, making it a more viable option for delivery systems with size limitations. The ‘miniSOX’ retains the potency of SOX17FNV with only 70% of its amino acids. A potential application of miniSOX in vivo could be in the emerging field of tissue regeneration and rejuvenation.

In preferred forms, the engineered miniSOX factor has the following amino acid sequence:
MSRIRRPMNAFMVWAKDERKRLAQQNPDLHNAELSKMLGKSWKALTFAEKRPFNEEAVR LRVQHMQDHPNYKYRPRRRKQVKRMKRVEGGFLHALVEPQAGALGPEGGRVAMDGLGLP FPEPGYPAGPPLMSPHMGPHYRDCQGLGAPALDGYPLPTPDTSPLDGVEQDPAFFAAPL PGDCPAAGTYTYAPVSDYAVSVEPPAGPMRVGPDPSGPAMPGILAPPSALHLYYGAMGS PAASAGRGFHAQPQQPLQPQAELLGEVDRTEFEQYLPFVYKPEMGLPYQGHDCGVNL (miniSOX; SEQ ID NO:2), or an amino acid sequence having at least 70%, at least 75%, at least 80%, at least 85%, at least 90%, at least 95%, at least 96%, at least 97%, at least 98%, at least 99%, or 100% sequence identify with miniSOX of SEQ ID NO:2, or a fragment thereof. Position 57 is indicated in bold and italic text. Mutations of position 53 to N and position 46 to F are also indicated in bold text.

An exemplary nucleic acid sequence for the minimal eSOX17 coding sequence is:

(miniSOX DNA; SEQ ID NO: 3)
ATGTCTCGCATCCGGCGGCCGATGAACGCCTTTATGGTGT
GGGCCAAAGACGAACGCAAGCGGTTGGCACAGCAGAACCC
AGATCTGCACAACGCAGAGCTAAGCAAGATGCTAGGCAAG
TCTTGGAAGGCGTTGACCTTTGCAGAGAAGCGGCCCTTCA
ATGAAGAGGCCGTGCGGCTGCGCGTGCAGCATATGCAGGA
CCACCCCAACTACAAGTACCGGCCGCGGCGGCGCAAGCAG
GTGAAGCGCATGAAGCGGGTGGAGGGAGGCTTCCTGCACG
CTCTCGTCGAGCCCCAGGCCGGCGCGCTTGGTCCCGAGGG
CGGCCGCGTGGCCATGGATGGCCTGGGTCTGCCTTTCCCG
GAGCCGGGCTATCCGGCCGGTCCTCCGCTGATGTCTCCGC
ACATGGGCCCCCACTATCGGGACTGCCAGGGACTGGGCGC
TCCCGCGCTCGACGGCTACCCTCTGCCCACTCCGGACACA
TCCCCGCTGGATGGCGTGGAGCAGGACCCGGCTTTCTTTG
CAGCCCCGCTGCCAGGGGACTGCCCGGCGGCCGGCACCTA
CACTTACGCTCCAGTCTCGGACTATGCAGTGTCCGTAGAG
CCGCCCGCTGGCCCCATGCGAGTGGGGCCGGACCCCTCGG
GCCCTGCGATGCCGGGGATCCTGGCGCCCCCCAGCGCTCT
GCACCTGTACTACGGCGCGATGGGCTCGCCCGCCGCAAGT
GCGGGGCGCGGTTTCCACGCGCAACCCCAGCAGCCGCTGC
AACCGCAGGCAGAGCTCCTAGGGGAGGTGGACCGCACGGA
ATTCGAACAGTATCTGCCCTTTGTGTATAAGCCCGAGATG
GGTCTTCCCTACCAGGGACACGACTGCGGAGTGAACCTCT
AA.

In some forms, an Engineered SOX17 factor (eSOX17) includes:
(i) the amino acid sequence of SEQ ID NO:1 or SEQ ID NO:2, or an amino acid sequence having at least 70%, 75%, 80%, 85%, 90%, 95%, or 99% sequence identity to SEQ ID NO:1, or SEQ ID NO:2, or a fragment variant thereof; or (ii) a nucleic acid encoding a polypeptide having the amino acid sequence of SEQ ID NO:1 or SEQ ID NO:2, or an amino acid sequence having at least 70%, 75%, 80%, 85%, 90%, 95%, or 99% sequence identity to SEQ ID NO:1, or SEQ ID NO:2, or a fragment variant thereof.

In some forms, the engineered minimal SOX polypeptide is a variant having an amino acid sequence having at least 95% sequence identity to SEQ ID NO:2. In some forms, the engineered minimal SOX polypeptide is a variant of the miniSOX polypeptide including a non-acidic amino acid such as alanine (A), arginine (R), asparagine (N), cysteine (C), glutamine (Q), glycine (G), histidine (H), isoleucine (I), leucine (L), lysine (K), methionine (M), phenylalanine (F), serine (S), threonine (T), tryptophan (W), and tyrosine (Y), instead of the naturally occurring glutamate residue at position 57 of the HMG box of the wild-type SOX17 protein. Therefore, engineered minimal SOX polypeptides having an amino acid sequence including any of these substitutions are provided. Mutations of position 57 are indicated in bold and italic text.

An exemplary polypeptide sequence of miniSOX17E57A includes:

(SEQ ID NO: 38)
MSRIRRPMNAFMVWAKDERKRLAQQNPDLHNAELSKMLGK
SWKALTLAEKRPFVEEAARLRVQHMQDHPNYKYRPRRRKQ
VKRMKRVEGGFLHALVEPQAGALGPEGGRVAMDGLGLPFP
EPGYPAGPPLMSPHMGPHYRDCQGLGAPALDGYPLPTPDT
SPLDGVEQDPAFFAAPLPGDCPAAGTYTYAPVSDYAVSVE
PPAGPMRVGPDPSGPAMPGILAPPSALHLYYGAMGSPAAS
AGRGFHAQPQQPLQPQAELLGEVDRTEFEQYLPFVYKPEM
GLPYQGHDCGVNL;

An exemplary polypeptide sequence of miniSOX17E57R includes:

(SEQ ID NO: 39)
MSRIRRPMNAFMVWAKDERKRLAQQNPDLHNAELSKMLGKSWKALTLAEK
RPFVEEARRLRVQHMQDHPNYKYRPRRRKQVKRMKRVEGGFLHALVEPQA
GALGPEGGRVAMDGLGLPFPEPGYPAGPPLMSPHMGPHYRDCQGLGAPAL
DGYPLPTPDTSPLDGVEQDPAFFAAPLPGDCPAAGTYTYAPVSDYAVSVE
PPAGPMRVGPDPSGPAMPGILAPPSALHLYYGAMGSPAASAGRGFHAQPQ
QPLQPQAELLGEVDRTEFEQYLPFVYKPEMGLPYQGHDCGVNL;

An exemplary polypeptide sequence of miniSOX17E57N includes:

(SEQ ID NO: 40)
MSRIRRPMNAFMVWAKDERKRLAQQNPDLHNAELSKMLGKSWKALTLAEK
RPFVEEANRLRVQHMQDHPNYKYRPRRRKQVKRMKRVEGGFLHALVEPQA
GALGPEGGRVAMDGLGLPFPEPGYPAGPPLMSPHMGPHYRDCQGLGAPAL
DGYPLPTPDTSPLDGVEQDPAFFAAPLPGDCPAAGTYTYAPVSDYAVSVE
PPAGPMRVGPDPSGPAMPGILAPPSALHLYYGAMGSPAASAGRGFHAQPQ
QPLOPQAELLGEVDRTEFEQYLPFVYKPEMGLPYQGHDCGVNL;

An exemplary polypeptide sequence of miniSOX17E57C includes:

(SEQ ID NO: 41)
MSRIRRPMNAFMVWAKDERKRLAQQNPDLHNAELSKMLGKSWKALTLAEK
RPFVEEACRLRVQHMQDHPNYKYRPRRRKQVKRMKRVEGGFLHALVEPQA
GALGPEGGRVAMDGLGLPFPEPGYPAGPPLMSPHMGPHYRDCQGLGAPAL
DGYPLPTPDTSPLDGVEQDPAFFAAPLPGDCPAAGTYTYAPVSDYAVSVE
PPAGPMRVGPDPSGPAMPGILAPPSALHLYYGAMGSPAASAGRGFHAQPQ
QPLQPQAELLGEVDRTEFEQYLPFVYKPEMGLPYOGHDCGVNL;

An exemplary polypeptide sequence of miniSOX17E57Q includes:

(SEQ ID NO: 42)
MSRIRRPMNAFMVWAKDERKRLAQQNPDLHNAELSKMLGKSWKALTLAEK
RPFVEEAQRLRVQHMQDHPNYKYRPRRRKQVKRMKRVEGGFLHALVEPQA
GALGPEGGRVAMDGLGLPFPEPGYPAGPPLMSPHMGPHYRDCQGLGAPAL
DGYPLPTPDTSPLDGVEQDPAFFAAPLPGDCPAAGTYTYAPVSDYAVSVE
PPAGPMRVGPDPSGPAMPGILAPPSALHLYYGAMGSPAASAGRGFHAQPQ
QPLQPQAELLGEVDRTEFEQYLPFVYKPEMGLPYQGHDCGVNL;

An exemplary polypeptide sequence of miniSOX17E57G includes:

(SEQ ID NO: 43)
MSRIRRPMNAFMVWAKDERKRLAQQNPDLHNAELSKMLGKSWKALTLAEK
RPFVEEAGRLRVQHMQDHPNYKYRPRRRKQVKRMKRVEGGFLHALVEPQA
GALGPEGGRVAMDGLGLPFPEPGYPAGPPLMSPHMGPHYRDCQGLGAPAL
DGYPLPTPDTSPLDGVEQDPAFFAAPLPGDCPAAGTYTYAPVSDYAVSVE
PPAGPMRVGPDPSGPAMPGILAPPSALHLYYGAMGSPAASAGRGFHAQPQ
QPLQPQAELLGEVDRTEFEQYLPFVYKPEMGLPYQGHDCGVNL;

An exemplary polypeptide sequence of miniSOX17E57H includes:

(SEQ ID NO: 44)
MSRIRRPMNAFMVWAKDERKRLAQQNPDLHNAELSKMLGKSWKALTLAEK
RPFVEEAHRLRVQHMQDHPNYKYRPRRRKQVKRMKRVEGGFLHALVEPQA
GALGPEGGRVAMDGLGLPFPEPGYPAGPPLMSPHMGPHYRDCQGLGAPAL
DGYPLPTPDTSPLDGVEQDPAFFAAPLPGDCPAAGTYTYAPVSDYAVSVE
PPAGPMRVGPDPSGPAMPGILAPPSALHLYYGAMGSPAASAGRGFHAQPQ
QPLQPQAELLGEVDRTEFEQYLPFVYKPEMGLPYQGHDCGVNL;

An exemplary polypeptide sequence of miniSOX17E57I includes:

(SEQ ID NO: 45)
MSRIRRPMNAFMVWAKDERKRLAQQNPDLHNAELSKMLGKSWKALTLAEK
RPFVEEAIRLRVQHMQDHPNYKYRPRRRKQVKRMKRVEGGFLHALVEPQA
GALGPEGGRVAMDGLGLPFPEPGYPAGPPLMSPHMGPHYRDCQGLGAPAL
DGYPLPTPDTSPLDGVEQDPAFFAAPLPGDCPAAGTYTYAPVSDYAVSVE
PPAGPMRVGPDPSGPAMPGILAPPSALHLYYGAMGSPAASAGRGFHAQPQ
QPLQPQAELLGEVDRTEFEQYLPFVYKPEMGLPYQGHDCGVNL;

An exemplary polypeptide sequence of miniSOX17E57L includes:

(SEQ ID NO: 46)
MSRIRRPMNAFMVWAKDERKRLAQQNPDLHNAELSKMLGKSWKALTLAEK
RPFVEEALRLRVQHMQDHPNYKYRPRRRKQVKRMKRVEGGFLHALVEPQA
GALGPEGGRVAMDGLGLPFPEPGYPAGPPLMSPHMGPHYRDCQGLGAPAL
DGYPLPTPDTSPLDGVEQDPAFFAAPLPGDCPAAGTYTYAPVSDYAVSVE
PPAGPMRVGPDPSGPAMPGILAPPSALHLYYGAMGSPAASAGRGFHAQPQ
QPLQPQAELLGEVDRTEFEQYLPFVYKPEMGLPYQGHDCGVNL;

An exemplary polypeptide sequence of miniSOX17E57K includes:

(SEQ ID NO: 47)
MSRIRRPMNAFMVWAKDERKRLAQQNPDLHNAELSKMLGKSWKALTLAEK
RPFVEEAKRLRVQHMQDHPNYKYRPRRRKQVKRMKRVEGGFLHALVEPQA
GALGPEGGRVAMDGLGLPFPEPGYPAGPPLMSPHMGPHYRDCQGLGAPAL
DGYPLPTPDTSPLDGVEQDPAFFAAPLPGDCPAAGTYTYAPVSDYAVSVE
PPAGPMRVGPDPSGPAMPGILAPPSALHLYYGAMGSPAASAGRGFHAQPQ
QPLQPQAELLGEVDRTEFEQYLPFVYKPEMGLPYQGHDCGVNL;

An exemplary polypeptide sequence of miniSOX17E57M includes:

(SEQ ID NO: 48)
MSRIRRPMNAFMVWAKDERKRLAQQNPDLHNAELSKMLGKSWKALTLAEK
RPFVEEAMRLRVQHMQDHPNYKYRPRRRKQVKRMKRVEGGFLHALVEPQA
GALGPEGGRVAMDGLGLPFPEPGYPAGPPLMSPHMGPHYRDCOGLGAPAL
DGYPLPTPDTSPLDGVEQDPAFFAAPLPGDCPAAGTYTYAPVSDYAVSVE
PPAGPMRVGPDPSGPAMPGILAPPSALHLYYGAMGSPAASAGRGFHAQPQ
QPLQPQAELLGEVDRTEFEQYLPFVYKPEMGLPYQGHDCGVNL;

An exemplary polypeptide sequence of miniSOX17E57F includes:

(SEQ ID NO: 49)
MSRIRRPMNAFMVWAKDERKRLAQQNPDLHNAELSKMLGKSWKALTLAEK
RPFVEEAFRLRVQHMQDHPNYKYRPRRRKQVKRMKRVEGGFLHALVEPQA
GALGPEGGRVAMDGLGLPFPEPGYPAGPPLMSPHMGPHYRDCQGLGAPAL
DGYPLPTPDTSPLDGVEQDPAFFAAPLPGDCPAAGTYTYAPVSDYAVSVE
PPAGPMRVGPDPSGPAMPGILAPPSALHLYYGAMGSPAASAGRGFHAQPQ
QPLQPQAELLGEVDRTEFEQYLPFVYKPEMGLPYQGHDCGVNL;

An exemplary polypeptide sequence of miniSOX17E57S includes:

(SEQ ID NO: 50)
MSRIRRPMNAFMVWAKDERKRLAQQNPDLHNAELSKMLGKSWKALTLAEK
RPFVEEASRLRVQHMQDHPNYKYRPRRRKQVKRMKRVEGGFLHALVEPQA
GALGPEGGRVAMDGLGLPFPEPGYPAGPPLMSPHMGPHYRDCQGLGAPAL
DGYPLPTPDTSPLDGVEQDPAFFAAPLPGDCPAAGTYTYAPVSDYAVSVE
PPAGPMRVGPDPSGPAMPGILAPPSALHLYYGAMGSPAASAGRGFHAQPQ
QPLQPQAELLGEVDRTEFEQYLPFVYKPEMGLPYQGHDCGVNL;

An exemplary polypeptide sequence of miniSOX17E57T includes:

(SEQ ID NO: 51)
MSRIRRPMNAFMVWAKDERKRLAQQNPDLHNAELSKMLGKSWKALTLAEK
RPFVEEATRLRVQHMQDHPNYKYRPRRRKQVKRMKRVEGGFLHALVEPQA
GALGPEGGRVAMDGLGLPFPEPGYPAGPPLMSPHMGPHYRDCQGLGAPAL
DGYPLPTPDTSPLDGVEQDPAFFAAPLPGDCPAAGTYTYAPVSDYAVSVE
PPAGPMRVGPDPSGPAMPGILAPPSALHLYYGAMGSPAASAGRGFHAQPQ
QPLQPQAELLGEVDRTEFEQYLPFVYKPEMGLPYQGHDCGVNL;

An exemplary polypeptide sequence of miniSOX17E57W includes:

(SEQ ID NO: 52)
MSRIRRPMNAFMVWAKDERKRLAQQNPDLHNAELSKMLGKSWKALTLAEK
RPFVEEAWRLRVQHMQDHPNYKYRPRRRKQVKRMKRVEGGFLHALVEPQA
GALGPEGGRVAMDGLGLPFPEPGYPAGPPLMSPHMGPHYRDCQGLGAPAL
DGYPLPTPDTSPLDGVEQDPAFFAAPLPGDCPAAGTYTYAPVSDYAVSVE
PPAGPMRVGPDPSGPAMPGILAPPSALHLYYGAMGSPAASAGRGFHAQPQ
QPLQPQAELLGEVDRTEFEQYLPFVYKPEMGLPYQGHDCGVNL;

An exemplary polypeptide sequence of miniSOX17E57Y includes:

(SEQ ID NO: 53)
MSRIRRPMNAFMVWAKDERKRLAQQNPDLHNAELSKMLGKSWKALTLAEK
RPFVEEAYRLRVQHMQDHPNYKYRPRRRKQVKRMKRVEGGFLHALVEPQA
GALGPEGGRVAMDGLGLPFPEPGYPAGPPLMSPHMGPHYRDCQGLGAPAL
DGYPLPTPDTSPLDGVEQDPAFFAAPLPGDCPAAGTYTYAPVSDYAVSVE
PPAGPMRVGPDPSGPAMPGILAPPSALHLYYGAMGSPAASAGRGFHAQPQ
QPLQPQAELLGEVDRTEFEQYLPFVYKPEMGLPYQGHDCGVNL;

and

An exemplary polypeptide sequence of miniSOX17E57V includes:

(SEQ ID NO: 54)
MSRIRRPMNAFMVWAKDERKRLAQQNPDLHNAELSKMLGKSWKALTLAEK
RPFVEEAVRLRVQHMQDHPNYKYRPRRRKQVKRMKRVEGGFLHALVEPQA
GALGPEGGRVAMDGLGLPFPEPGYPAGPPLMSPHMGPHYRDCQGLGAPAL
DGYPLPTPDTSPLDGVEQDPAFFAAPLPGDCPAAGTYTYAPVSDYAVSVE
PPAGPMRVGPDPSGPAMPGILAPPSALHLYYGAMGSPAASAGRGFHAQPQ
QPLQPQAELLGEVDRTEFEQYLPFVYKPEMGLPYQGHDCGVNL

### 2. Vectors

It will be appreciated that eSOX17 can be administered to cells or a subject as a nucleic acid encoding protein (transcribed RNA, DNA, DNA in an expression vector). For example, a sequence encoding eSOX17 can be incorporated into an autonomously replicating plasmid, a virus (e.g., a retrovirus, lentivirus, adenovirus, or herpes virus), or into the genomic DNA of a prokaryote or eukaryote. In some forms, the transcription factor cocktail of OCT4, eSOX17 (such as SOX17FNV, an example described herein), KLF4 and C-MYC are administered to cells or a subject as a nucleic acid encoding each transcription factor on the same vector or different vectors. In some forms, the nucleic acids encoding each transcription factor are integrated into the genome of the target or donor cells, for example, via a lentiviral system. In preferred forms, the vectors are integration-free systems, such as episomal vector, minicircle vectors, plasmids, protein expression vectors, integration-free lentiviral vectors and Sendai viral system.

In some forms, an eSOX17 is encoded within a lentivirus vector. For example, in some forms, an eSOX17 polypeptide, such as eSOX17FNV, or miniSOX is encoded within nucleic acid of a lentivirus vector configured for expression of the eSOX in a recipient target cell. In some forms, the lentiviral vector is also configured to express one or more additional factors in a host cell, in addition to the eSOX17. For example, in some forms, the lentiviral vector is configured to over-express one or more of KLF4, C-MYC, and/or OCT4 in addition to eSOX17 in the host cell. Exemplary Dox-inducible lentiviral particles encoding human KLF4, C-MYC and eSOX17FNV (K/M/eSOX17FNV).

An exemplary lentivirus particle is a Dox-inducible lentiviral particle that also encodes human KLF4, and/or C-MYC. In other forms, two or more vectors are combined in a composition. For example, in some forms, one or more lentiviral vector(s) that is configured to over-express one or more of KLF4, C-MYC, and/or OCT4 in a host cell is combined in a composition with lentiviral vector that is configured to over-express eSOX17 in the same host cell.

In some forms, a lentiviral encoding an engineered SOX17 factor (eSOX17) includes:
(i) a nucleic acid encoding a polypeptide including the amino acid sequence of SEQ ID NO:1 or SEQ ID NO:2, or an amino acid sequence having at least 70%, 75%, 80%, 85%, 90%, 95%, or 99% sequence identity to SEQ ID NO:1, or SEQ ID NO:2, or a fragment variant thereof; and
(ii) a nucleic acid encoding a human KLF4, and/or C-MYC.

### C. Source of Cells

The iEPSCs can be obtained by reprogramming human somatic cells, or partially or completely differentiated cells obtained from a mammal such as any mammal (e.g., bovine, ovine, porcine, canine, feline, equine, primate), preferably a human. Sources include bone marrow, fibroblasts, fetal tissue (e.g., fetal liver tissue), peripheral blood, umbilical cord blood, urine, pancreas, skin or any organ or tissue.

In some forms, the iEPSCs are derived from human somatic cells. A somatic cell as would be understood by one of ordinary skill in the art is any cell other than a gamete (sperm or egg), germ cell (cells that go on to become gametes), gametocyte or undifferentiated stem cell.

The somatic cells can be obtained from tissue such as bone marrow, fetal tissue (e.g., fetal liver tissue), peripheral blood, umbilical cord blood, pancreas, skin or any organ or tissue. In one form, the iEPSCs are derived from fibroblasts, adipose-derived cells, neural cells, or cells from the intestinal epithelium. In another form, iEPSCs are obtained from induced neonatal (for example foreskin) or adult fibroblasts. However, iEPSCs can be obtained from other cell types including but not limited to somatic cells of hematological origin, skin derived cells, adipose cells, epithelial cells, endothelial cells, cells of mesenchymal origin, parenchymal cells (for example, hepatocytes), neurological cells, and connective tissue cells.

In preferred forms, the iEPSCs are effectively generated from rare tissue specimen such as small tissue biopsies or a few drops of blood.

In some forms, cells are isolated by disaggregating an appropriate organ or tissue which is to serve as the cell source using techniques known to those skilled in the art. For example, the tissue or organ can be disaggregated mechanically and/or treated with digestive enzymes and/or chelating agents that weaken the connections between neighboring cells, so that the tissue can be dispersed to form a suspension of individual cells without appreciable cell breakage. Enzymatic dissociation can be accomplished by mincing the tissue and treating the minced tissue with one or more enzymes such as trypsin, chymotrypsin, collagenase, elastase, and/or hyaluronidase, DNase, pronase, dispase etc. Mechanical disruption can also be accomplished by a number of methods including, but not limited to, the use of grinders, blenders, sieves, homogenizers, or pressure cells.

D. Formulations of iEPSCs

Cells derived from the iEPSCs can be formulated for administration, delivery or contacting with a subject, tissue, or cell to promote de-differentiation in vivo, in vitro, or ex vivo, as well as served as personalized disease modelling and in vitro platform for drug screening. Additional factors, such as growth factors, other factors that induce differentiation or dedifferentiation, secretion products, immunomodulators, anti-inflammatory agents, regression factors, biologically active compounds that promote innervation, vascularization or enhance the lymphatic network, and drugs, can be incorporated.

The induced totipotent-like stem cells can be administered to a patient by way of a composition that includes a population of iEPSCs or progeny thereof alone or on or in a carrier or support structure. In many embodiments, no carrier will be required. The cells can be administered by injection onto or into the site where the cells are required. In these cases, the cells will typically have been washed to remove cell culture media and will be suspended in a physiological buffer.

In other forms, the cells are provided with or incorporated onto or into a support structure. Support structures may be meshes, solid supports, scaffolds, tubes, porous structures, and/or a hydrogel. Such solid supports and methods of culturing cells thereon are known in the art.

### III. Methods of Making

It has been established that engineered SOX17 (eSOX17) factors can substantially enhance the reprogramming efficiency of human induced expanded potential stem cells (iEPSCs) which represent a state reminiscent to totipotent cells. Compared to conventionally used SOX2, eSOX17 converts cells more rapidly and efficiently and even reprograms recalcitrant cells such as high passage senescent fibroblasts where SOX2 fails. It has also been established that, when administered to a cell in combination with other specific factors, reprogramming with engineered SOX17 bypasses pluripotency to induce tri-potent neural stem cells (iNSCs).

A. Methods of Generating Human iEPSCs

Methods of reprogramming human somatic cells to provide induced expanded potential stem cells (iEPSCs) are described. It has been established that the combination of over-expressed eSOX17FNV with other transcription factors can drive parent somatic cells to alternative cell fates upon exposure to extrinsic factors such as expanded potential stem cell medium (EPSCM) for pluripotency. Therefore, methods to engineer somatic cells to express one or more markers of pluripotency, including OCT4, SOX2, NANOG, and LIN28 are provided.

In some forms, the methods involve the steps of:
(i) introducing an engineered SOX17 together with one or more additional exogenous transcription factors to the donor cells such as somatic cells; and
(ii) culturing the population in an expanded potential stem cell medium (EPSCM).

### 1. Introducing Exogenous Transcription Factor(s)

The methods include one or more steps of introducing engineered SOX17, together with one or more other exogenous transcription factors into a somatic cell. Typically, the methods for providing iEPSCs include the step of exogenously introducing the engineered SOX17 factor along with one or more of OCT4, KLF4, and C-MYC into one or more somatic cells. The expression can be induced for example by forced gene expression or using RNAs, non-integrating gene expression vectors, or proteins. For example, in some forms, the methods deliver engineered SOX17 factor, OCT4, KLF4, and C-MYC to one or more somatic cells using lentiviral system. In a preferred form, methods deliver engineered SOX17 factor, OCT4, KLF4, and C-MYC to one or more somatic cells using non-integrating gene expression vectors, such as episomal vectors.

### 2. Culturing in an Expanded Potential Stem Cell Medium (EPSCM)

In some forms, the methods further include one or more steps of culturing donor cells having exogenously introduced transcription factors, such as the engineered SOX17, OCT4, KLF4, and C-MYC, in an Expanded Potential Stem Cell Medium (EPSCM). Therefore, in some forms, the methods contact the transgenic donor cells having exogenously introduced transcription factors including the engineered SOX17, OCT4, KLF4, and C-MYC with an EPSCM for an amount of time and under conditions suitable to produce a population of induced expanded potential stem cells (iEPSCs).

Exemplary EPSCM includes one or more of a Ras-ERK inhibitor, a Src Kinase family (SFK) inhibitor, a GSK3 inhibitor, a Wnt inhibitor, and optionally LIF, IGF-II or Activin or its functional equivalent, to produce a population of EPSCs.

In other forms, the EPSCM further includes one or more of a Jun N-Terminal Kinase (JNK) inhibitor, and/or a p38 inhibitor. Thus, in one form, the EPSCM includes a RAS-ERK inhibitor, a Src Kinase family (SFK) inhibitor, a GSK3 inhibitor, a Wnt inhibitor, a p38 inhibitor, a JNK inhibitor to produce a population of EPSCs.

B. Methods of Generating Human iNSCs

Methods of reprogramming human somatic cells to provide induced neural stem cells (iNSCs) are described. It has been established that certain combination of over-expressed eSOX17FNV with other transcription factors does not lead to the acquisition of pluripotency, such that eSOX17FNV can drive parent somatic cells to alternative cell fates with additional transcription factors and upon exposure to extrinsic conditions, such as Leukemia inhibitory factor (LIF), and neural stem cell medium NSCM for iNSCs.

Therefore, methods to engineer somatic cells to express one or more markers of neural stem cells, including SOX1, SOX2, PAX6 and FABP7 are provided.

In some forms, the methods involve the steps of:
(i) introducing an engineered SOX17, optionally together with one or more additional exogenous transcription factors to the donor cells such as somatic cells; and
(ii) culturing the population in a neural stem cell medium (NSCM).

### 1. Introducing Exogenous Transcription Factor(s)

The methods include one or more steps of introducing engineered SOX17, together with one or more other exogenous transcription factors into a somatic cell.

Typically, methods for providing iNSCs include the step of exogenously introducing the engineered SOX17 factor into one or more somatic cells. In some forms, the methods also introduce exogenous KLF4, and/or C-MYC into the same somatic cells.

As demonstrated in the Examples, iNSCs reprogrammed with eSOX17FNV within 2 and 3 factor cocktails do not transition to pluripotency. Methods to produce eSOX17-derived iNSCs include introducing reprogramming factors in cells, including KLF4 (K) and/or C-MYC (M), which are over-expressed together with an eSOX17, such as eSOX17FNV. In exemplary methods, M and eSOX17FNV are introduced into somatic cells under conditions suitable for the over-expression of these factors, relative to expression of these factors in wild-type cells. In some methods, C-MYC, KLF4 and eSOX17FNV are overexpressed in the transgenic cells. In other methods, eSOX17FNV and KLF4 are overexpressed in the transgenic cells. In other methods, only eSOX17FNV is overexpressed in cells. In other forms, eSOX17FNV and C-MYC together with one or more additional factors are overexpressed in cells.

The expression of factors in somatic cells can be induced, for example, by forced gene expression or using RNAs, non-integrating gene expression vectors, or proteins. For example, in some forms, the methods deliver an engineered SOX17, KLF4, and/or C-MYC to one or more somatic cells using lentiviral system. In a preferred form, methods deliver an engineered SOX17, KLF4, and/or C-MYC to one or more somatic cells using non-integrating gene expression vectors, such as episomal vectors.

### 2. Culturing in a Neural Stem Cell Medium (NSCM)

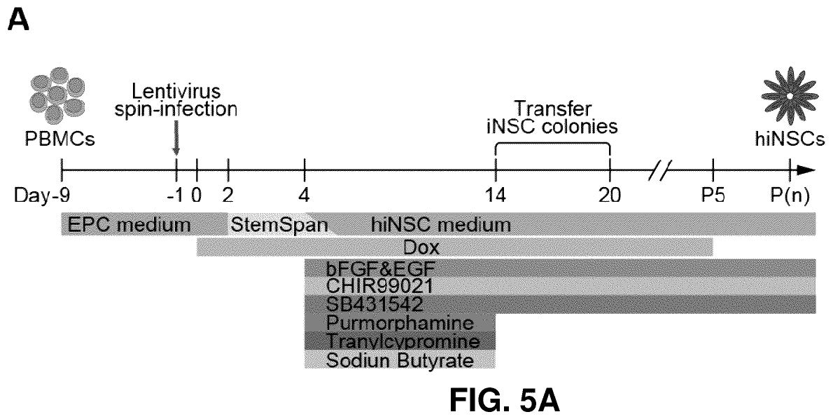

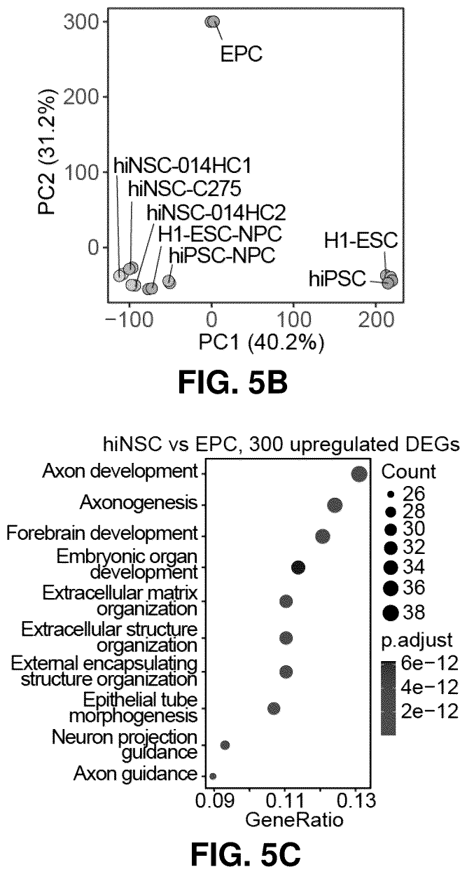

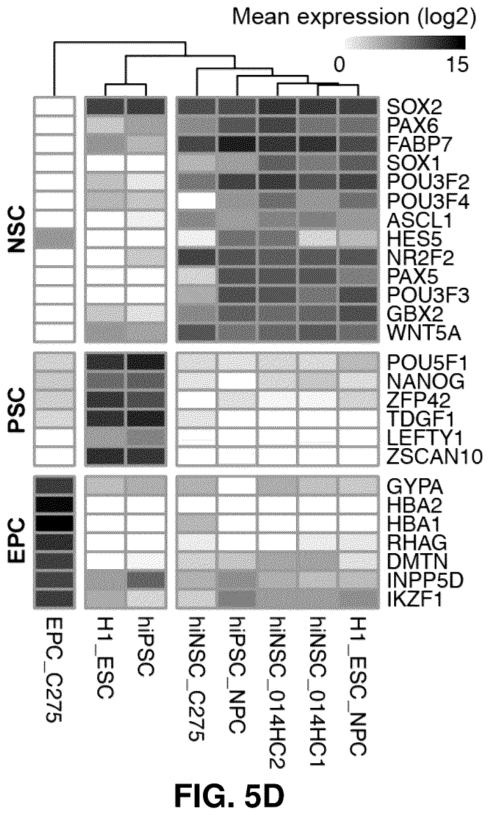

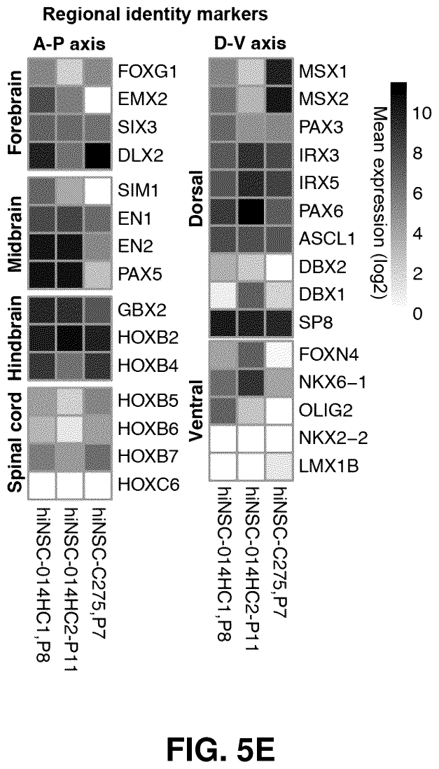

In some forms, the methods generate eSOX17-derived iNSCs. As described in the Examples, after omitting individual factors from the KLF4, C-MYC and eSOX17FNV (K/M/eSOX17FNV) 3-factor cocktail, it was found that C-MYC and eSOX17FNV (M/eSOX17FNV) are sufficient for iNSC conversion. Transduced EPCs formed adherent, elongated cells within 4 days and NSC-like colonies within 11-20 days. In some forms, the methods include one or more steps to expand cells or colonies of iNSCs, including culturing cells in neural stem cell medium (NSCM) (FIG. 5A).

In some forms, the methods further include one or more steps of culturing donor cells having exogenously introduced transcription factors, such as the engineered SOX17 and C-MYC, in neural stem cell medium (NSCM). Therefore, in some forms, the methods contact the transgenic donor cells having exogenously introduced transcription factors including the engineered SOX17 and C-MYC with an NSCM for an amount of time and under conditions suitable to produce a population of induced neural stem cells (iNSCs).

In some forms, the methods contact the transgenic donor cells having exogenously introduced transcription factors including the engineered SOX17 with a NSCM including one or more of a GSK3 inhibitor, a TGF-β inhibitor, LIF, bFGF and EGF to produce a population of neural stem cells (NSCs). LIF is a secreted cytokine, plays an important role in a wide array of biological processes including inducing differentiation of leukemia cell, inflammatory response, neuronal development, embryonic implantation, stem cell self-renewal and cancer progression, etc.

C. Isolation of iEPSCs and iNSCs

Media that can maintain the undifferentiated state and totipotency of iEPSCs or iNSCs or induce differentiation are known in this field. Differentiation and proliferation abilities of isolated induced totipotent-like stem cells can be easily confirmed by those skilled in the art by using confirmation means widely applied to stem cells.

A substantially purified population of iEPSCs or iNSCs can be obtained, for example, by picking dome-shaped colonies from a culture source. Purity can be measured by any appropriate method. Human iEPSCs or iNSCs can be isolated by, for example, utilizing molecules (e.g., antibodies, antibody derivatives, ligands, or Fc-peptide fusion molecules) that bind to a marker on the induced totipotent stem cells or iNSCs and thereby positively selecting cells that bind the molecule (i.e., a positive selection). Other examples of positive selection methods include methods of preferentially promoting the growth of a desired cell type in a mixed population of desired and undesired cell types. Alternatively, by using molecules that bind to markers that are not present on the desired cell type, but that are present on an undesired cell type, the undesired cells containing such markers can be removed from the desired cells (i.e., a negative selection). Other negative selection methods include preferentially killing or inhibiting the growth of an undesired cell type in a mixed population of desired and undesired cell types. Accordingly, by using negative selection, positive selection, or a combination thereof, an enriched population of stem cell can be made.

Procedures for separation may include magnetic separation, using antibody-coated magnetic beads, affinity chromatography, cytotoxic agents joined to a monoclonal antibody, or such agents used in conjunction with a monoclonal antibody, e.g., complement and cytotoxins, and “panning” with antibody attached to a solid matrix (e.g., plate), or other convenient technique. Techniques providing accurate separation include fluorescence activated cell sorters, which can have varying degrees of sophistication, e.g., a plurality of color channels, low angle and obtuse light scattering detecting channels, and impedance channels. Antibodies may be conjugated with markers, such as magnetic beads, which allow for direct separation, biotin, which can be removed with avidin or streptavidin bound to a support, or fluorochromes, which can be used with a fluorescence activated cell sorter, to allow for ease of separation of the particular cell type. Any technique may be employed which is not unduly detrimental to the viability of the induced pluripotent stem cells. In one embodiment, the cells are incubated with an antibody against a marker and the cells that stain positive for the marker are manually selected and sub-cultured.

Combinations of enrichment methods may be used to improve the time or efficiency of purification or enrichment. For example, after an enrichment step to remove cells having markers that are not indicative of the cell type of interest, the cells may be further separated or enriched by a fluorescence activated cell sorter (FACS) or other methodology having high specificity. Multi-color analyses may be employed with a FACS. The cells may be separated on the basis of the level of staining for a particular antigen or lack thereof. Fluorochromes may be used to label antibodies specific for a particular antigen. Such fluorochromes include phycobiliproteins, e.g., phycoerythrin and allophycocyanins, fluorescein, and Texas red.

D. Culturing and Preservation of iEPSCs or iNSCs

In some forms, the iEPSCs are cultured and/or maintained in the Expanded Potential Stem Cell Medium (EPSCM). In some forms, the iNSCs are cultured and/or maintained in the NSCM.

In preferred forms, the iEPSCs or iNSCs are cultured and/or maintained under a feeder-free and xeno-free culturing condition suitable for providing clinical grade cells.

The iEPSCs or iNSCs can be expanded in culture and stored for later retrieval and use. Once a culture of cells or a mixed culture of stem cells is established, the population of cells is mitotically expanded in vitro by passage to fresh medium as cell density dictates under conditions conducive to cell proliferation. Such culturing methods can include, for example, passaging the cells in culture medium lacking particular growth factors that induce differentiation (e.g., human LIF and/or other growth factor). Cultured cells can be transferred to fresh medium when sufficient cell density is reached. Some stem cell types do not demonstrate typical contact inhibition-apoptosis or they become quiescent when density is maximum. Accordingly, appropriate passaging techniques can be used to reduce contact inhibition and quiescence.

Cells can be cryopreserved for storage according to known methods, such as those described in Doyle et al., (eds.), 1995, Cell & Tissue Culture: Laboratory Procedures, John Wiley & Sons, Chichester. For example, cells may be suspended in a “freeze medium” such as culture medium containing 50% fetal bovine serum (FBS) and 10% dimethylsulfoxide (DMSO), and 10 μM ROCK inhibitor Y-27632, at a density, for example, of about 4-10×10⁵ cells/ml. The cells are dispensed into cryogenic vials which are then sealed and transferred to a freezing chamber of a programmable or passive freezer. The optimal rate of freezing may be determined empirically. For example, a freezing program that gives a change in temperature of −1° C./min through the heat of fusion may be used. Once vials containing the cells have reached −80° C., they are transferred to a liquid nitrogen storage area. Cryopreserved cells can be stored for a period of years.

1. Feeder-Free iEPSCs

In some forms, stable iNSCs are generated and cultured as feeder-free cells. In some forms, stable iEPSC clones are cultured on feeder cells. In some other forms, stable iEPSC clones are cultured as feeder-free cells. Thus, stable feeder-free iEPSC clones and methods of making thereof are also described.

In one form, to adapt iEPSCs from feeder culture to feeder-free condition, iEPSCs grown on feeders are dissociated in 0.05% Trypsin/EDTA. In one form, to remove feeders, digested cell suspension is seeded on gelatinized plates for 45 min. In a further form, loosely-/non- attached iEPSCs are seeded on 12-well plate coated with 2×Matrigel-coated plates and cultured in feeder-free medium (provided by CTSCB). In another form, to passage feeder-free iEPSCs, when cells reach 80% confluency, iEPSCs are detached and dissociated into single cells, followed by seeding on Matrigel-coated plates. In preferred forms, to generate stable feeder-free iEPSC clones, cells are sub-cultured for more than 5 passages.

2. Differentiation of iEPSCs or iNSC

The described eSOX17-derived iEPSCs may subsequently be differentiated into other cell types using techniques which are known in the art. The described eSOX17-derived iNSCs may subsequently be differentiated into neurons, astrocytes and oligodendrocytes in vitro and/or in vivo, using techniques which are known in the art.

i. Culture and Differentiation of iEPSCs

In one form, the iEPSCs are differentiated into trophoblasts. Techniques for differentiating totipotent-like stem cells into trophoblasts are known. Thus, the iEPSCs may be differentiated into trophoblasts by culturing the EPSCs in a trophoblast induction medium to induce trophoblast differentiation. The induction medium may be a TGFβ inhibitor-containing medium. Treatment of TGFβ inhibitor SB431542 to EPSC has been reported to generate trophoblast-like cells (Gao X, et al., Nat Cell Biol. 2019;21(6):687-99). Thus, in some forms, iEPSCs are induced to express one or more of the trophoblast-related marker genes including CDX2, GATA3, TRAP2C, KRT8, KRT18, and HAND1.

Human blastoids provide a readily accessible, scalable, versatile, and perdurable alternative to blastocysts for studying early human development, understanding early pregnancy loss, and gaining insights into early developmental defects. The iEPSCs can be differentiated to generate blastocyst-like structures in vitro, ‘blastoids’ which resemble human blastocysts in terms of their morphology, size, cell number, and composition and allocation of different cell lineages. In some forms, iEPSCs are induced to express one of more of the blastocyst and trophoblast related marker genes including CDX2, GATA3, KRT8, KRT18, and TRAP2C.

ii. Culture and Differentiation of iNSCs

In some forms, the methods include one or more steps to expand cells or colonies of iNSCs, including culturing cells in neural stem cell medium (NSCM) supplemented with bFGF, EGF, CHIR99021, and SB431542. In some forms, the iNSCs are differentiated into neurons, astrocytes and oligodendrocytes. Techniques for differentiating tri-potent neural stem cells into neurons, astrocytes and oligodendrocytes are known. In one form, the iNSCs may be differentiated into generic neurons to express one or more neuron marker genes including TUJ1, NF, MAP2, SYN1 and PSD95, TH and vGLUT2. In one form, the iNSCs may be differentiated into motor neurons to express one or more motor neuron marker genes including HB9 and ISL1. In one form, the iNSCs may be differentiated into astrocytes to express one or more astrocyte marker genes including GFAP and S100B. In one form, the iNSCs may be differentiated into oligodendrocytes to express one or more oligodendrocyte marker genes including O4 and OLIG2.

### IV. Methods of Use

Methods of using the described eSOX17-derived Induced Expanded Potential Stem Cells (iEPSCs) and compositions thereof of are described. In has been established that eSOX17-derived iEPSCs can give rise to a desired cell type or morphology. Therefore, in some forms, methods of eSOX17-derived iEPSCs provide therapeutic treatments, tissue engineering and/or research. In some forms, the methods employ eSOX17-derived iEPSCs for transplantation, tissue engineering, regulation of angiogenesis, vasculogenesis, and/or cell replacement or cell therapies. In other forms, the methods employ the described eSOX17-derived iEPSCs and/or compositions thereof for the treatment and/or prevention of certain diseases and disorders. In other forms, iEPSCs are used to introduce a gene into a subject as part of a gene therapy regimen.

In some forms, the methods use eSOX17-derived iEPSCs for study of human diseases and/or disorders. In exemplary forms, the methods employ eSOX17-derived iEPSCs in the study of reproductive human disorders, such as preeclampsia. For example, in some forms, the methods employ eSOX17-derived iEPSCs as cell models for implantation and early human development without embryo destruction. In further forms, the eSOX17-derived iEPSCs are used in methods to provide preclinical potential in disease modelling, for example, for use in ameliorating reproductive human disorders such as preeclampsia or uterine disorders such as endometriosis.

### A. Providing Cells for Studying Early Human Development

In some forms, eSOX17-derived iEPSCs are used in methods for studying early human development. The study of human development from fertilized egg to mature embryo is extremely important but the early differentiation of human tissues remains an enigma. Moreover, the relatively high percentage of unexplained pregnancy loss emphasizes the need for an appropriate model for studying early human development. In some forms, the iEPSCs disclosed are particularly suited for use in previously inaccessible basic processes that occur during human embryogenesis, such as gastrulation and organogenesis.

It was shown that iEPSCs can be induced into human blastoids, which mimic the stages before the embryo implants into the uterus. The use of iEPSC for such studies would obviate the need for human embryos and usage of ethically challenging cell sources. Thus, in some forms, iEPSCs or induced human blastoids thereof are used in cell models in the preclinical study of infertility-related diseases such as infertility, failure of implantations, and preeclampsia.

### B. Providing Differentiated Somatic Cells (Re-Differentiated Cells)

In some forms, eSOX17-derived iEPSCs are used in methods for providing differentiated somatic cells having a desired phenotype. Methods including re-differentiation of eSOX17-derived iEPSCs are provided.

Once established, a culture of totipotent-like stem cells may be used to produce progeny cells, for example, neurons or fibroblasts capable of producing new tissue. In some forms, the iEPSCs is induced to differentiate into one or more cells from any of the three germ layers, for example, skin and hair cells including epithelial cells, keratinocytes, melanocytes, adipocytes, cells forming bone, muscle and connective tissue such as myocytes, chondrocytes, osteocytes, alveolar cells, parenchymal cells such as hepatocytes, renal cells, adrenal cells, and islet cells (e.g., alpha cells, delta cells, PP cells, and beta cells), blood cells (e.g., leukocytes, erythrocytes, macrophages, and lymphocytes), retinal cells (and other cells involved in sensory perception, such as those that form hair cells in the ear or taste buds on the tongue), and nervous tissue including nerves.

In one form, the iEPSCs are induced to differentiate into cells of ectodermal origin by exposing the cells to an “ectodermal differentiating” media. In another form, the iEPSCs are induced to differentiate into cells of mesodermal origin by exposing the cells to “mesodermal differentiating media”. In still another form, the iEPSCs are induced to differentiate into cells of endodermal origin by exposing the cells to “endodermal media”. Components of “endodermal”, “mesodermal” and “ectodermal” media are known to one of skill in the art. Known cell surface markers can be used to verify that the cells are indeed differentiating into cells of the lineage of the corresponding cell culture medium. The most commonly accepted markers to confirm differentiation of the three germ layers are the expression of alpha fetal protein for endodermal cells, alpha smooth muscle actin for mesoderm, and Beta-III tubulin for ectoderm, all of which are normally expressed very early in the development of these tissues.

Differentiation of stem cells to fibroblasts or other cell types, followed by the production of tissue therefrom, can be triggered by specific exogenous growth factors or by changing the culture conditions (e.g., the density) of a stem cell culture. Methods for inducing differentiation of cells into a cell of a desired cell type are known in the art. For example, iEPSCs can be induced to differentiate by adding a substance (e.g., a growth factor, enzyme, hormone, or other signaling molecule) to the cell's environment. Examples of factors that can be used to induce differentiation include erythropoietin, colony stimulating factors, e.g., GM-CSF, G-CSF, or M-CSF, interleukins, e.g., IL-1, -2, -3, -4, -5, -6, -7, -8, or Leukemia Inhibitory Factory (LIF), coculture with tissue committed cells, or other lineage committed cells types to induce the stem cells into becoming committed to a particular lineage.

The re-differentiated cells can be expanded in culture and stored for later retrieval and use.

### C. Cell Therapy

Methods of using Esox17-derived iEPSCs for cell therapy are provided. Therapeutic uses of the induced totipotent-like stem cells include transplanting the iEPSCs, or progeny thereof into individuals to treat a variety of pathological states including diseases and disorders resulting from cancers, wounds, neoplasms, injury, viral 15 infections, diabetes, and the like. Treatment may entail the use of the cells to produce new tissue, and the use of the tissue thus produced, according to any method presently known in the art or to be developed in the future. The cells may be implanted, injected, or otherwise administered directly to the site of tissue damage so that they will produce new tissue in vivo. In one form, administration includes the administration of genetically modified iEPSCs or their progeny.

In a preferred form, the iEPSCs are obtained from autologous cells i.e., the donor cells are autologous. However, the cells can be obtained from heterologous cells. In one form, the donor cells are obtained from a donor genetically related to the recipient. In another form, donor cells are obtained from a donor genetically un-related to the recipient.

If the totipotent-like iEPSCs are derived from a heterologous (non-autologous/allogenic) source compared to the recipient subject, concomitant immunosuppression therapy is typically administered, e.g., administration of the immunosuppressive agent cyclosporine or FK506. However, due to the immature state of the human induced totipotent stem cells such immunosuppressive therapy may not be required. Accordingly, in one form, the human iEPSCs can be administered to a recipient in the absence of immunomodulatory (e.g., immunosuppressive) therapy. Alternatively, the cells can be encapsulated in a membrane, which permits exchange of fluids but prevents cell/cell contact. Transplantation of microencapsulated cells is known in the art, e.g., Balladur et al., Surgery, 117:189-94, 1995; and Dixit et al., Cell Transplantation 1:275-79 (1992).

### 1. Diabetes

In some forms, eSOX17-derived iEPSCs are used for the treatment and/or prevention of Diabetes mellitus (DM) in a subject in need thereof. Diabetes mellitus is a group of metabolic diseases where the subject has high blood sugar, either because the pancreas does not produce enough insulin, or, because cells do not respond to insulin that is produced.

A promising replacement for insulin therapy is provision of islet cells to the patient in need of insulin. Shapiro et al., N Engl J Med., 343(4):230-8 (2000) have demonstrated that transplantation of beta cells/islets provides therapy for patients with diabetes. Although numerous insulin types are commercially available, these formulations are provided as injectables. The human induced totipotent-like stem cells, iEPSCs, provide an alternative source of islet cells to prevent or treat diabetes. For example, iEPSCs can be isolated and differentiated to a pancreatic cell type and delivered to a subject. Alternatively, the iEPSCs can be delivered to the pancreas of the subject and differentiated to islet cells in vivo. Accordingly, the cells are useful for transplantation in order to prevent or treat the occurrence of diabetes. Methods for reducing inflammation after cytokine exposure without affecting the viability and potency of pancreatic islet cells are disclosed for example in U.S. Patent No. 8,637,494 to Naziruddin, et al.

2. Neurodegenerative disorders

In some forms, eSOX17-derived iEPSCs and iNSCs are used for the treatment and/or prevention of neurodegenerative diseases and disorders in a subject in need thereof.

Neurodegenerative disorders are characterized by conditions involving the deterioration of neurons as a result of disease, hereditary conditions or injury, such as traumatic or ischemic spinal cord or brain injury. Neurodegenerative conditions include any disease or disorder or symptoms or causes or effects thereof involving the damage or deterioration of neurons. Neurodegenerative conditions can include, but are not limited to, Alexander Disease, Alper's Disease, Alzheimer Disease, Amyotrophic Lateral Sclerosis, Ataxia Telangiectasia, Canavan Disease, Cockayne Syndrome, Corticobasal Degeneration, Creutzfeldt-Jakob Disease, Huntington Disease, Kennedy's Disease, Krabbe Disease, Lewy Body Dementia, Machado-Joseph Disease, Multiple Sclerosis, Parkinson Disease, Pelizaeus-Merzbacher Disease, Niemann-Pick's Disease, Primary Lateral Sclerosis, Refsum's Disease, Sandhoff Disease, Schilder's Disease, Steele-Richardson-Olszewski Disease, Tabes Dorsalis or any other condition associated with damaged neurons. Other neurodegenerative conditions can include or be caused by traumatic spinal cord injury, ischemic spinal cord injury, stroke, traumatic brain injury, and hereditary conditions.

In particular, the disclosed methods include transplanting into a subject in need thereof neural stem cells, neural progenitors, or neural precursors that have been expanded in vitro such that the cells can ameliorate the neurodegenerative condition. Transplantation of the expanded neural stem cells can be used to improve ambulatory function in a subject suffering from various forms of myelopathy with symptoms of spasticity, rigidity, seizures, paralysis, or any other hyperactivity of muscles. Methods for expanding and transplanting neural cells and neural progenitor cells for the treatment of different neurodegenerative conditions is disclosed for example, in U.S. Pat. No. 8,236,299 to Johe, et al.

### 3. Cancer Therapy

In some forms, eSOX17-derived iEPSCs are used for the treatment and/or prevention of cancer and/or proliferative diseases and disorders in a subject in need thereof.

Therapeutic uses of the iEPSCs and their progeny include transplanting the induced totipotent-like stem cells or progeny thereof into individuals to treat and/or ameliorate the symptoms associated with cancer. For example, in one form, the progeny of iEPSCs can be administered to cancer patients who have undergone chemotherapy that has killed, reduced, or damaged cells of a subject. In a typical stem cell transplant for cancer, very high doses of chemotherapy are used, often along with radiation therapy, to try to destroy all the cancer cells. This treatment also kills the stem cells in the bone marrow. Soon after treatment, stem cells are given to replace those that were destroyed.

In another form, the iEPSCs can be transfected or transformed (in addition to the de-differentiation factors) with at least one additional therapeutic factor. For example, once iEPSCs are isolated, the cells may be transformed with a polynucleotide encoding a therapeutic polypeptide and then implanted or administered to a subject, or may be differentiated to a desired cell type and implanted and delivered to the subject. Under such conditions the polynucleotide is expressed within the subject for delivery of the polypeptide product.

### 4. Tissue Engineering

In some forms, eSOX17-derived iEPSCs are used for tissue engineering. iEPSCs and their progeny can be used to make tissue engineered constructions, using methods known in the art. Tissue engineered constructs may be used for a variety of purposes including as prosthetic devices for the repair or replacement of damaged organs or tissues. They may also serve as in vivo delivery systems for proteins or other molecules secreted by the cells of the construct or as drug delivery systems in general. Tissue engineered constructs also find use as in vitro models of tissue function or as models for testing the effects of various treatments or pharmaceuticals. The most commonly used biomaterial scaffolds for transplantation of stem cells are reviewed in the most commonly used biomaterial scaffolds for transplantation of stem cells is reviewed in Willerth, S. M. and Sakiyama-Elbert, S. E., Combining stem cells and biomaterial scaffolds for constructing tissues and cell delivery (Jul. 9, 2008), StemBook, ed. The Stem Cell Research Community, StemBook. Tissue engineering technology frequently involves selection of an appropriate culture substrate to sustain and promote tissue growth. In general, these substrates should be three-dimensional and should be processable to form scaffolds of a desired shape for the tissue of interest.

U.S. Pat. No. 6,962,814 generally discloses method for producing tissue engineered constructs and engineered native tissue. With respect to specific examples, U.S. Pat. No. 7,914,579 to Vacanti, et al., discloses tissue engineered ligaments and tendons. U.S. Pat. No. 5,716,404 discloses methods and compositions for reconstruction or augmentation of breast tissue using dissociated muscle cells implanted in combination with a polymeric matrix. U.S. Pat. No. 8,728,495 discloses repair of cartilage using autologous dermal fibroblasts. U.S. Published application No. 20090029322 by Duailibi, et al., discloses the use of stem cells to form dental tissue for use in making tooth substitute. U.S. Published application No. 2006/0019326 discloses cell-seed tissue-engineered polymers for treatment of intracranial aneurysms. U.S. Published application No. 2007/0059293 by Atala discloses the tissue-engineered constructs (and method for making such constructs) that can be used to replace damaged organs for example kidney, heart, liver, spleen, pancreas, bladder, ureter, and urethra.

### D. Personalized Disease Modeling

In some forms, eSOX17-derived iEPSCs are used for the modeling of diseases and disorders in a subject in need thereof.

Given the enhanced efficiency of engineered SOX17 factor (SOX17FNV as an example in this invention) to generate human iEPSCs and the capacity to generate human blastoids, Human blastoids can be used as cell-based model to understand the genetic, cellular and molecular mechanisms of infertility-related disorders.

### E. Drug Screening Platform

In some forms, eSOX17-derived iEPSCs are used for the screening of agents that have activity, such as therapeutic activity against one or more diseases and disorders.

The establishment of totipotent-like stem cells open the avenue to generate the cell types of interest for high throughput screening. For example, to screen for immunomodulatory compounds, T lymphocytes can be generated from the established differentiation protocol and adopted as screening platform to identify immunosuppressive compounds for organ transplantation and immunostimulatory compounds for cancer therapy and anti-viral therapy. In other forms, human blastoids generated from iEPSCs can be served as high-throughput platform in drug discovery to prevent pregnancy failure and birth defects and development of contraceptive drugs.

### V. Kits

Kits are also disclosed. The kits can include, for example, combination of transcription factors including engineered SOX17 factor, OCT4, KLF4, and C-MYC suitable for generating human induced expanded potential stem cells (iEPSCs). In some forms, the kits also include suitable medium for exogenously introducing one or more transcription factors into somatic cells, and/or medium for culturing reprogrammed cells. In further forms, the kits contain the engineered SOX17-derived iEPSCs suitable for clinical study or use in treatment as well as for basic research of early human development. The kits can include printed instructions for administering the compound in a use as described above.

The described compositions and methods will be better understood with reference to the following paragraphs:
1. A method for reprogramming human somatic cells into human induced expanded potential stem cells (iEPSCs), the method including:
(i) introducing one or more exogenous transcription factors to the human somatic cells, wherein the one or more exogenous transcription factors include an engineered SOX17 factor, and
(ii) culturing the population in an expanded potential stem cell medium (EPSCM).
2. The method of paragraph 1, wherein the engineered SOX17 factor has the amino acid sequence of SEQ ID NO:1, or a variant thereof.
3. The method of paragraph 1 or 2, wherein the one or more exogenous transcription factors further include OCT4, KLF4, and C-MYC.
4. The method of any one of paragraphs 1-3, wherein the one or more exogenous transcription factors are encoded as nucleic acids.
5. The method of paragraph 4, wherein the nucleic acids encoding the one or more exogenous transcription factors are incorporated into a vector.
6. The method of paragraph 5, wherein the vector is an integration-free system selected from the group including episomal vectors, minicircle vectors, and sendai viral vectors.
7. The method of paragraph 5, wherein the vector is a viral vector selected from the group including retrovirus, lentivirus, adenovirus, or herpes virus.
8. The method of any one of paragraphs 1-7, wherein the EPSCM includes one or more of a RAS-ERK inhibitor, a Src Kinase family (SFK) inhibitor, a GSK3 inhibitor, a Wnt inhibitor, a p38 inhibitor, and a JNK inhibitor.
9. The method of any one of paragraphs 1-8, wherein the EPSC-like colonies are produced in less than 4 days after culturing in EPSCM.
10. The method of paragraph 9, further including the step of picking EPSC-like colonies to generate iEPSC clone from single cell.
11. The method of any one of paragraphs 1-10, further including culturing iEPSC to feeder-free condition.
12. The method of any one of paragraphs 1-11, further including expanding iEPSC for more than 20 passages, for example, for up to 25 passages in cell culture.
13. The method of any one of paragraphs 1-12, further including differentiating iEPSC into trophoblasts or blastoids.
14. The method of any one of paragraphs 1-13, wherein the human somatic cells are selected from the group including cells of hematological origin, skin derived cells, adipose cells, epithelial cells, endothelial cells, cells of mesenchymal origin, parenchymal cells, neurological cells, and connective tissue cells.
15. The method of paragraph 14, wherein the human somatic cells are dermal fibroblasts or peripheral blood cells.
16. The method of any one of paragraphs 1-15, wherein the human somatic cells are reprogramming-resistant cells such as high passage senescent fibroblasts.
17. The method of any one of paragraphs 1-16, wherein the human somatic cells are reprogramming-resistant cells where OCT4, KLF4, and C-MYC in combination with SOX2 fails to induce pluripotency.
18. Human totipotent-like stem cells obtained by a method according to any one of paragraphs 1-17, characterized in that they express at least the core pluripotency transcriptional factors engineered SOX17, OCT4, KLF4, and C-MYC.
19. The human totipotent-like stem cells of paragraph 18, wherein the human totipotent-like stem cells are cryopreserved.
20. A method of using the human totipotent-like stem cells of paragraph 18 or 19 for studying early human development.
21. A method for reprogramming human somatic cells into human induced totipotent or multipotent stem cells, the method comprising:
(i) introducing one or more exogenous transcription factors to the human somatic cells to provide reprogrammed cells,
wherein one exogenous transcription factor comprises an engineered SOX17 factor (eSOX17), and
(ii) culturing the reprogrammed cells in a stem cell medium to provide induced totipotent or multipotent cells, and
(iii) optionally isolating the induced totipotent or multipotent stem cells.
22. The method of paragraph 21, wherein the eSOX17 comprises the amino acid sequence of SEQ ID NO:1, or an amino acid sequence comprising at least 70%, 75%, 80%, 85%, 90%, 95%, or 99% sequence identity to SEQ ID NO:1, or a fragment thereof.
23. The method of paragraph 21, wherein the eSOX17 comprising the amino acid sequence of SEQ ID NO:2, or an amino acid sequence having at least 70%, 75%, 80%, 85%, 90%, 95%, or 99% sequence identity to SEQ ID NO:2, or a fragment variant thereof.
24. The method of paragraph 21, wherein the eSOX17 comprises the amino acid sequence of any one of SEQ ID NOs:1-2 or 4-54.
25. The method of any one of paragraphs 21-24, wherein the one or more exogenous transcription factors further comprises one or more of OCT4, KLF4, and C-MYC, and
wherein the stem cell medium comprises an expanded potential stem cell medium (EPSCM).
26. The method of paragraph 21, wherein the eSOX17 comprises the amino acid sequence of any one of SEQ ID NO:1-2 or SEQ ID NO:4-54,
wherein the exogenous transcription factors further comprise one or more of KLF4 and C-MYC, and
wherein the stem cell medium comprises a neural stem cell medium (NSCM).
27. The method of any one of paragraphs 21-26, wherein the one or more exogenous transcription factors are encoded as nucleic acids, optionally wherein the nucleic acids are encoded and/or expressed by a vector.
28. The method of any one of paragraphs 21-27, wherein the vector is an integration-free system selected from the group consisting of episomal vectors, minicircle vectors, and Sendai viral vectors.
29. The method of any one of paragraphs 21-27, wherein the vector is a viral vector selected from the group consisting of retrovirus, lentivirus, adenovirus, or herpes virus.
30. The method of any one of paragraphs 21-29, wherein the EPSCM comprises one or more of a RAS-ERK inhibitor, a Src Kinase family (SFK) inhibitor, a GSK3 inhibitor, a Wnt inhibitor, a p38 inhibitor, and a JNK inhibitor.
31. The method of any one of paragraphs 21-30, wherein the induced totipotent or multipotent stem cells are isolated in less than 8 days, less than 7 days, less than 6 days, less than 5 days, or less than 4 days after culturing in stem cell medium.
32. The method of any one of paragraphs 21-31, wherein culturing the reprogrammed cells comprises culturing in the absence of feeder cells.
33. The method of any one of paragraphs 21-32 further comprising expanding the induced totipotent or multipotent stem cells for at least 20 passages and up to 42 passages in cell culture.
34. The method of any one of paragraphs 21-33 further comprising, after step (ii),
culturing the induced totipotent cells in a trophoblast induction medium to induce differentiation of the cells into trophoblasts, or
culturing the induced totipotent cells in a blastoid induction medium to induce differentiation of the cells into blastoids.
35. The method of any one of paragraphs 21-34, wherein the human somatic cells are selected from the group consisting of cells of hematological origin, skin derived cells, adipose cells, epithelial cells, endothelial cells, cells of mesenchymal origin, parenchymal cells, neurological cells, and connective tissue cells.
36. The method of paragraph 35, wherein the human somatic cells are dermal fibroblasts or peripheral blood cells.
37. Human induced expanded potential stem cells (iEPSCs) obtained by the method of any one of paragraphs 21-36, optionally wherein the iEPSCs are frozen in a cryo-preservative.
38. Human induced neural stem cells (iNSC) obtained by the method of any one of paragraphs 21-36, optionally wherein the iNSCs are frozen in a cryo-preservative.
39. A method of using the induced totipotent or multipotent cells produced by the method of any one of paragraphs 21-36 for treating or preventing one or more disease or disorders in a subject in need thereof,
optionally wherein the one or more disease or disorders are selected from the group consisting of wounds, injuries, cancers, diabetes, and neurodegenerative diseases.
40. An engineered SOX17 factor (eSOX17) comprising:
(i) the amino acid sequence of SEQ ID NO:1-2 or SEQ ID NO: 4-54, or an amino acid sequence comprising at least 70%, 75%, 80%, 85%, 90%, 95%, or 99% sequence identity to SEQ ID NO:1-2, or SEQ ID NO:4-54, or a fragment variant thereof; or
(ii) a nucleic acid encoding a polypeptide comprising the amino acid sequence of SEQ ID NO:1-2 or SEQ ID NO:4-54, or an amino acid sequence comprising at least 70%, 75%, 80%, 85%, 90%, 95%, or 99% sequence identity to SEQ ID NO:1-2, or SEQ ID NO:4-54, or a fragment variant thereof.

The present disclosure is further understood by reference to the following non-limiting examples.

### EXAMPLES

### Example 1: Efficient Reprogramming of Human Cells

### Materials and Methods

### Generation of Lentivirus Particles for Reprogramming

Lentiviral particles were prepared as described with some modifications (Hockemeyer D, Soldner F, Cook E G, Gao Q, Mitalipova M, Jaenisch R. Cell Stem Cell. 2008;3(3):346-53). Briefly, 293 cells were seeded at 6×10⁶ cells per 100 mm dish and cultured overnight followed by incubation with 10 μg of expression plasmid (polycistronic lentiviral system FUW-tetO-hOKMS addgene #51543 and homemade OKMSOX17FNV by replacing SOX2 with engineered SOX17 factor SOX17FNV; FUW-M2rtTA addgene #20342) along with packaging plasmids (psPAX2 addgene #12260 and pMD2.G addgene #12259) in 1 mL Opti-MEM medium containing 60 μL transfection reagent polyethyleneimine (PEI, Polysciences, #23966). Transfection mixture was added to the cells and culture volume was adjusted to 10 mL DMEM (Thermo Fisher Scientific, #12100046) with 10% FBS (Thermo Fisher Scientific, #10270106). Culture medium was changed within 10-14 h to fresh DMEM supplemented with 10% FBS (1 mM sodium butyrate (Sigma, #B5887) can be added depending on the size of expression plasmid). Lentiviral particles containing supernatants were collected after 48 and 72 h and cell debris was removed by passing through 0.45 μm filters (Minisart, ·16537-K).

### EPSC Reprogramming

Human adult dermal fibroblasts (HDFa, #C0135C) and neonatal dermal fibroblasts (HDFn, #C0045C) were purchased from Thermo Fisher Scientific. Cells were seeded at 25000 cells per well of gelatinized 12-well plate and cultured in HFB medium composed of MEM (Thermo Fisher Scientific, #11-095-080) with 10% FBS, 1× GlutaMAX (Thermo Fisher Scientific, #35050061), 1× nonessential amino acids (NEAA; Thermo Fisher Scientific, #11140050), 0.1 mM 2-Mercaptoethanol (Thermo Fisher Scientific, #31350010) and 0.5× penicillin/streptomycin (P/S; Thermo Fisher Scientific, #15140-122). The day before lentiviral transfection, culture medium was changed to M10 medium composed of Knockout DMEM (Thermo Fisher Scientific, #10829018) with 10% FBS, 1× GlutaMAX, 1× NEAA and 0.5× P/S . Filtered lentiviral supernatant containing 8 μg/mL polybrene (Sigma-Aldrich, #40804ES76) of a polycistronic reprogramming 4-factor system encoding OKMS2 or OKMSOX17FNV was added to HDFs. After 24 hr of viral infection, medium was changed to M15 medium composed of knockout DMEM with 15% FBS, 1× GlutaMAX, 1× nonessential amino acids and 0.5× penicillin/streptomycin containing 0.5 ug/mL Doxycycline (Dox; Sigma, #D9891) and cultured for 2-3 days. Transduced HDFs were detached by 0.05% Trypsin (Thermo Fisher Scientific, #25300062) and counted for cell viability. Cells were the seed at 7500 cells per well of 12-well of mitomycin C-treated MEF-feeder plate and cultured in M15 medium with 0.5 ug/mL Dox. M15 medium was then changed to reprogramming medium (M15 medium containing lug/mL vitamin C, 1 ng/mL home-made human LIF with 1 ug/mL Dox) and this day is considered as day 0 of reprogramming. The reprogramming cells were maintained at 37° C. and 5% CO₂ and monitored using a phase-contrast microscope. Reprogramming medium with Dox was changed daily until day 14. EPSC-like colonies were firstly observed on day 6 (OKMSOX17FNV) and day 8 (OKMS2). Human EPSCM medium (provided by CTSCB) without Dox was then added to reprogrammed cells on day 14 and fresh EPSCM medium was changed daily until day 21. Dome-shaped EPSC colonies were identified 3-4 days after medium changing to EPSCM.

### Colony Picking and Passaging

To establish iEPSC colonies, EPSC colonies on MEF-feeder cell layer were picked between days 21 and 30 using sterile syringe needle and micropipette and transferred into a 1.5 mL centrifuge tube containing 0.05% Trypsin/EDTA to digest into single cell suspension. Digested cells were seeded on feeder cell layer in EPSCM and medium was changed daily. EPSC colonies with dome-shaped morphology were observed 3-5 days after cell seeding. Colony picking procedure as mentioned above was repeated twice to generate iEPSC clone originated from single cells. For cell passage of established iEPSC clones, when EPSC colonies reach 80% confluency, cells were washed with DPBS (Thermo Fisher Scientific, #14040133), then incubated with 0.05% Trypsin for 2 min at 37 degrees and neutralized with M10 medium. Digested cells were then seeded on feeder cell layer at the ratio of 1:3 to 1:5 and cultured in EPSCM.

Generation of Feeder-Free iEPSC Clones

To adapt iEPSCs from feeder culture to feeder-free condition, iEPSCs grown on feeders were dissociated in 0.05% Trypsin/EDTA. To remove feeders, digested cell suspension was seeded on gelatinized plates for 45 min. Loosely-/non-attached iEPSCs were seeded on 12-well plate coated with 2× Matrigel (Corning, #354234) and cultured in feeder-free medium (provided by CTSCB) with 10% KSR and 5μM Y27632 (MedChem Express, #HY-10583). To passage feeder-free iEPSCs, when cells reach 80% confluency, iEPSCs were detached by incubation in TryPLE (Thermo Fisher Scientific, #12-605-028) at 37 degrees for 2 min. Cells were neutralized with M10 medium and digested cell suspension was added to Matrigel-coated plates at the ratio of 1:5. To generate stable feeder-free iEPSC clones, cells were sub-cultured for more than 5 passages.

Differentiation of iEPSCs to Trophoblast Lineages

Human iEPSCs were dissociated with TrypLE and seeded on gelatinized 6-well plates. The cells were cultured in feeder-free medium (provided by CTSCB) with 20% KSR media and 5 μM Y27632 overnight. Trophoblast lineage differentiation was induced by adding 10 μM SB431542 (MedChem Express, #HY-10431). The cells were collected on day 6 for measurement of trophoblast-related gene expression (Gao X, et al., Nat Cell Biol. 2019;21(6):687-99).

Trophoblast Stem Cell (TSC) Derivation from hiEPSCs

Human iEPSCs were dissociated with TryPLE and seeded on gelatinized 12-well plates. The cells were cultured in trophoblast stem cell medium (TSCM) composed of DMEM/F12 (Thermo Fisher Scientific, #12-500-062) supplemented with 0.1 mM 2-mercaptoethanol (Thermo Fisher Scientific, #31350010), 0.2% FBS, 0.5% P/S, 0.3% BSA (Thermo Fisher Scientific, #15260037), 1% ITS-X supplement (Thermo Fisher Scientific, #51500056), 1.5 mg/ml L-ascorbic acid, 50 ng/ml EGF (PeproTech, #AF-100-15), 2 mM CHIR99021 (SelleckChem, #S2924), 0.5 mM A83-01 (Tocris, #2939), 1 mM SB431542, 0.8 mM Valproic acid (Sigma, #P4543), and 5 mM Y27632. TSCM was changed to every other day. When cells reach 80% confluency, cells were dissociated with TryPLE and passaged at the ratio of 1:5 in TSCM (Okae H, et al., Cell Stem Cell. 2018;22(1):50-63.e6).

Human iBlastoid Generation from Human iEPSCs.

Human iEPSCs were dissociated with TryPLE and seeded on low attachment 96 round-bottom plate at the cell density of 200 or 400 cells per well of plate). The cells were cultured in N2B27 medium composed of 1:1 of DMEM-F12: Neurobasal medium (Thermo Fisher Scientific, #21103049) with growth factors and supplements including 1× N2 (Thermo Fisher Scientific, #17502048), 1× B27 (Thermo Fisher Scientific, #17504044), 1× GlutaMAX, 1× nonessential amino acids, 0.1 mM 2-Mercaptoethanol and 1× penicillin/streptomycin and 10 μM Y27632 overnight. Medium was then changed to N2B27 medium with PALLY including PD0325901 (SelleckChem, #S1036), A83-01, 1-Oleoyl lysophosphatidic acid (LPA; MedChem Express, #HY-137862), human LIF and Y-27632) for 2 days with daily medium change. Cells were then cultured in N2B27 medium containing LPA and Y-27632 for another two days. Blastocyst-like structures were then observed, and images were captured with CKX53(Olympus) inverted microscope

mRNA Isolation and Real-Time qPCR

The iEPSCs after 5 passages were cultured in 12-well plates. Cells were harvested at 80% cell confluency. Culture medium was removed, washed with DPBS, and detached in 0.05% Trypsin-EDTA. RNA was extracted using the TRIzol (Thermo Fisher Scientific, #15596018). RNA was quantified using a Nanodrop spectrophotometer. Two micrograms of total mRNA were used to synthesize the cDNA with a ReverTra Ace qPCR RT master mix (Toybo, #FSQ-201s). qPCR was performed with iTaq Universal SYBR Green Supermix (Biorad, #1725122) on a CFX-96 thermocycler (Bio-Rad). Relative gene expression was calculated using the 2ΔΔCt method with Gapdh as endogenous control.

### Immunofluorescence

The iEPSCs were seeded on Matrigel-coated 24-well cell culture plates (for feeder free iEPSCs) or gelatinized 24-well cell culture plates with MEF feeder layers (for feeder iEPSCs) and grown for 2-3 days in EPSCM or feeder free medium until 80% confluence. Cells were washed with DPBS three times and fixed with 4% paraformaldehyde at room temperature for 15 min. Cells were permeabilized in 0.2% Triton X-100 (Sigma-Aldrich, #T8787) at room temperature for 10 min. Permeabilized cells were washed with DPBS twice and blocked with 10% BSA (United States Biological, #A1311) at room temperature for 1 hr. Primary antibodies were added to the cells and incubated at 4° C. overnight. The cells were then washed with DPBS three times and incubated with corresponding secondary antibodies at room temperature for 1 hr. Cells were washed with DPBS three times and counter-stained with lx DAPI (Thermo Fisher Scientific, #R37606). Images were captured with CKX53 (Olympus) inverted microscope.

### Results

### Engineered SOX17 Factor Dramatically Boosts Reprogramming Efficiency of Human iEPSCs from Dermal Fibroblast

An earlier study developed an approach termed directed evolution of reprogramming factors by cell selection and sequencing (DERBY-seq) to systematically identify engineered transcription factors with enhanced pluripotency function in somatic reprogramming. The engineered SOX17FNV is one of the engineered eSOX17 transcriptions factors identified from the high throughput screen on mouse fibroblast reprogramming (Veerapandian, et al., Stem Cell Reports. 2018;11(2):593-606). Culture conditions to derive and establish expanded or extended potential stem cells (EPSCs) from preimplantation embryos has been reported in different laboratories with high success rates (Gao, et al., Nat Cell Biol. 2019;21(6):687-99; and Yang, et al., Nature. 2017;550(7676):393-7). However, the generation of human induced EPSCs (iEPSCs) from somatic cells is still challenging due to the low reprogramming efficiency and requirement of additional co-factors (Liu, et al., Nat Commun. 2021;12(1):3017).

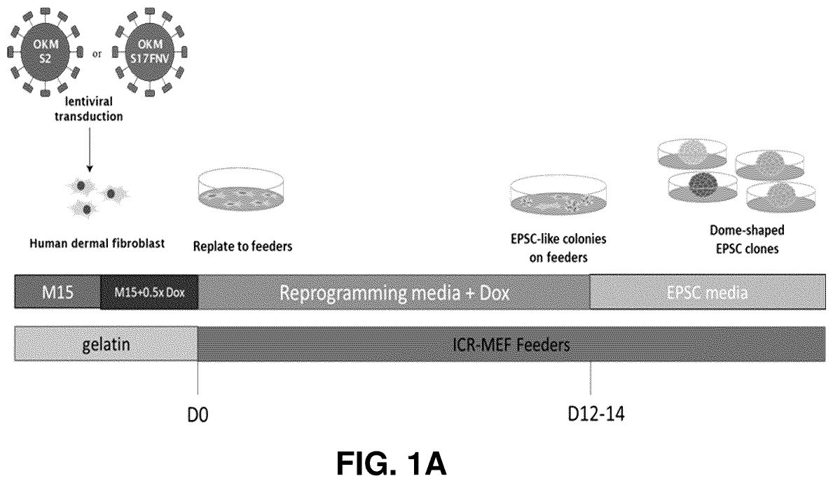

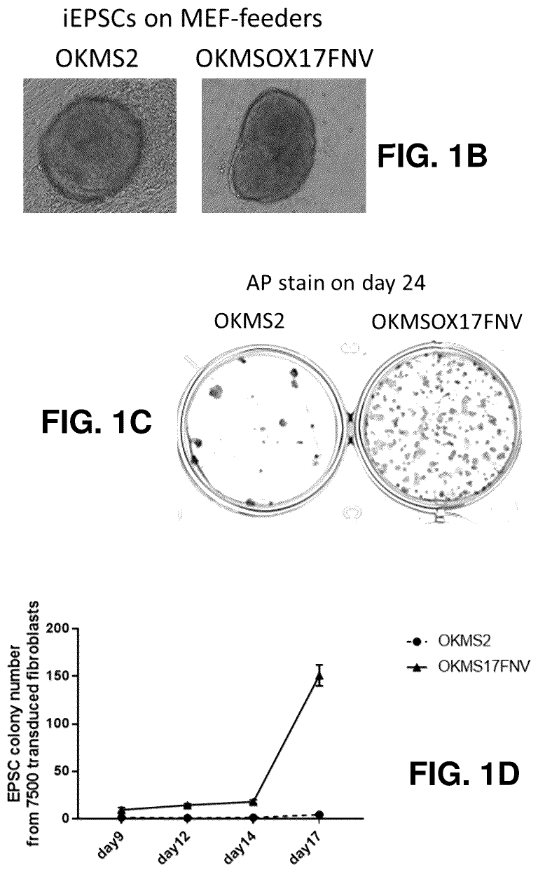

Given the findings that engineered factor SOX17FNV can substantially enhance mouse and human iPSC reprogramming from fibroblasts, a polycistronic lentiviral system was generated to reprogram human dermal fibroblasts by expressing Doxycycline (Dox)-inducible reprogramming factors (OKM-S2 and OKM-SOX17FNV) (FIG. 1A). EPSC-like colonies started to appear from day 6 of reprogramming in OKM-SOX17FNV group, which is 3-4 days earlier than OKM-S2 group. Dome-shaped iEPSCs were confirmed and identified on day 12-14 of reprogramming (FIG. 1B). It was found that SOX17FNV can substantially enhance the reprogramming efficiency of human iEPSCs to approximately 2%. This is ˜30 times higher than in the corresponding OKMS2 condition (FIG. 1C and 1D). Further, Cells from aged individuals as well as high-passage senescent cells are often difficult to be reprogramed using conventional SOX2. SOX17FNV seemed to also promote iEPSC reprogramming from human senescent fibroblasts at high passage whilst SOX2 could not (FIG. 1E). Taken together, the engineered transcription SOX17 factor SOX17FNV can enhance the speed and efficiency of EPSC reprogramming from human dermal fibroblasts and empowers the generation of iEPSCs from cell sources where SOX2 fails.

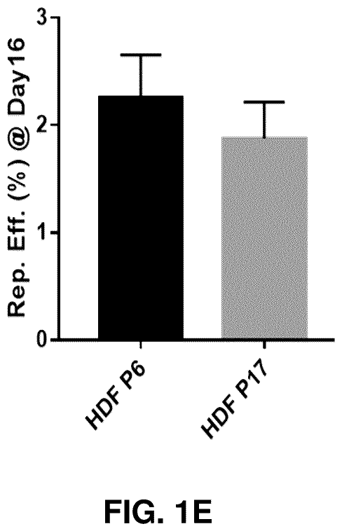

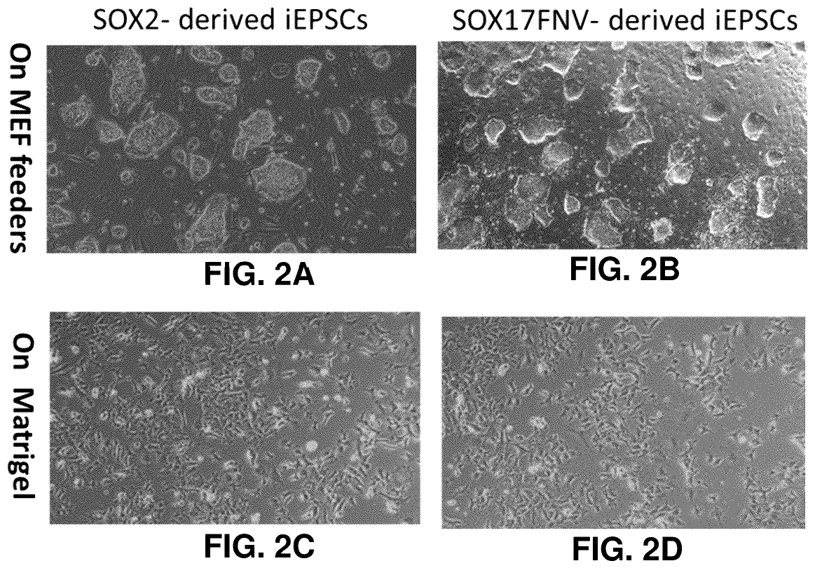

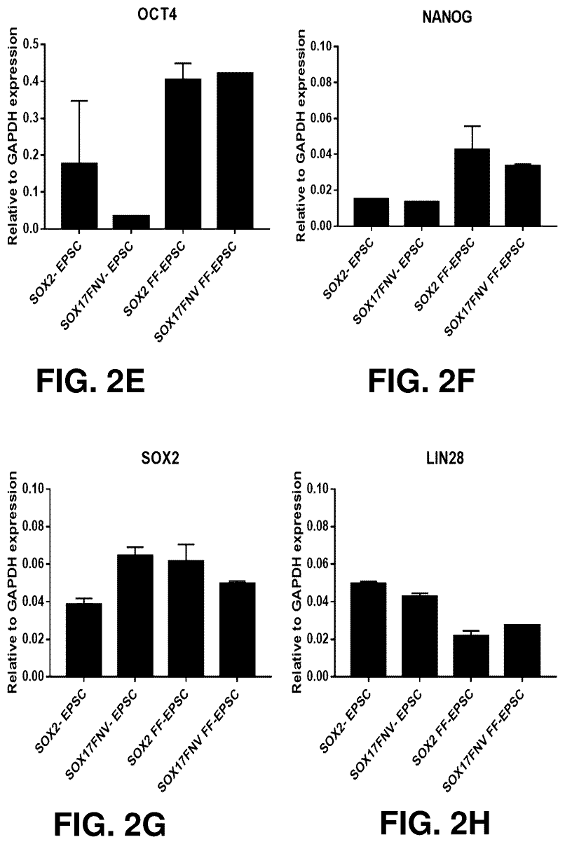

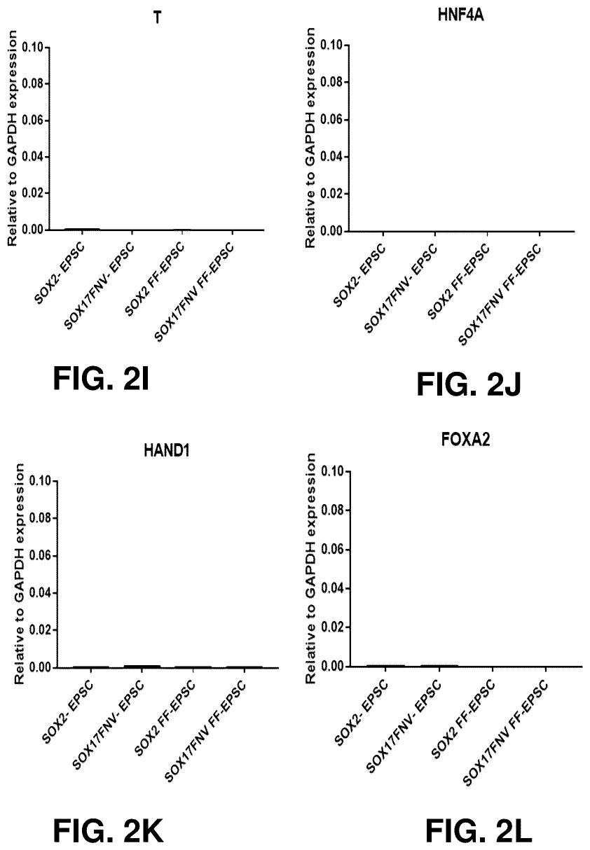

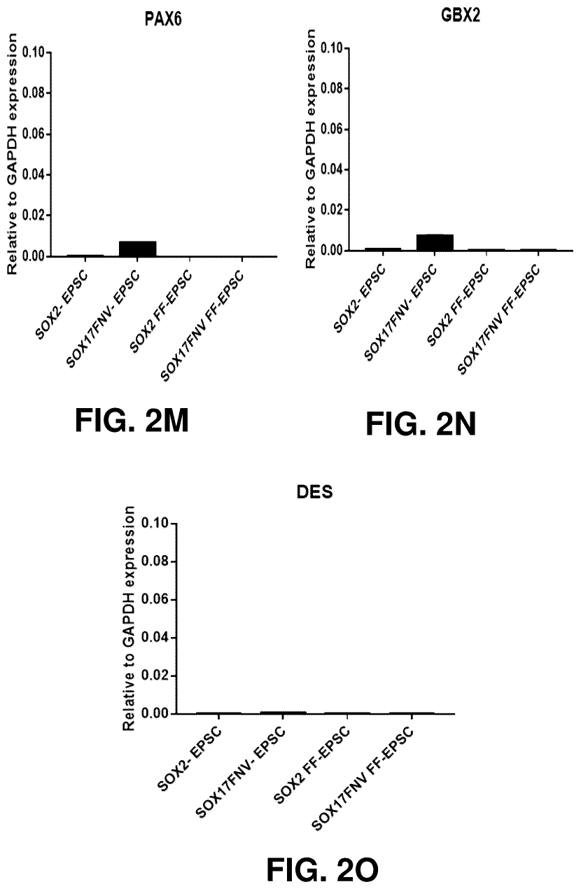

Establishment of human induced EPSCs in feeder and feeder-free conditions The generated human iEPSCs were next validated by establishing EPSC clones from single cells. On MEF feeder condition, human SOX2- and SOX17FNV-derived iEPSC clones can be passaged for more than 20 passages without differentiation. These human iEPSCs clones showed pluripotent signature by expressing panel of pluripotency makers evaluated by qPCR (FIG. 2B) and immunostaining. These iEPSC clones were further adapted to feeder-free condition (Matrigel) to generate feeder free clones. These feeder-free clones can be passaged for more than 15 passages without differentiation. Pluripotency signatures can be retained in these feeder free iEPSC clones (FIGS. 2A-2O).

### Human iEPSCs Show Differentiation Potential to Trophoblasts and TSC Derivation from Human iEPSCs

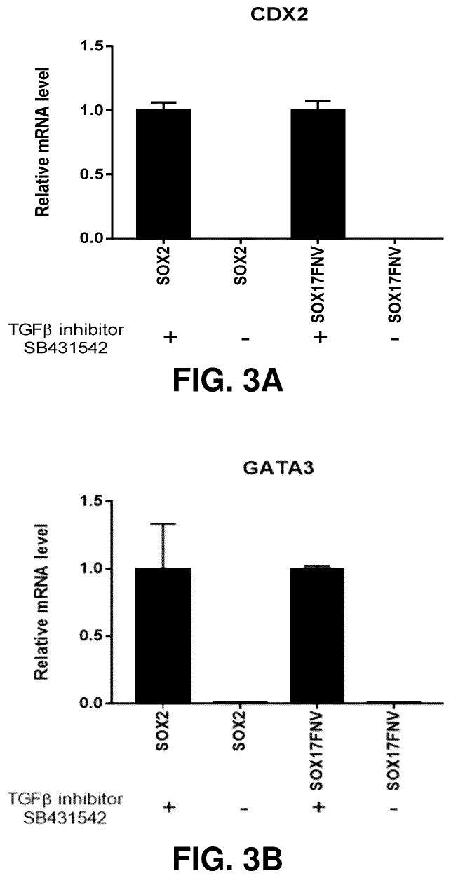

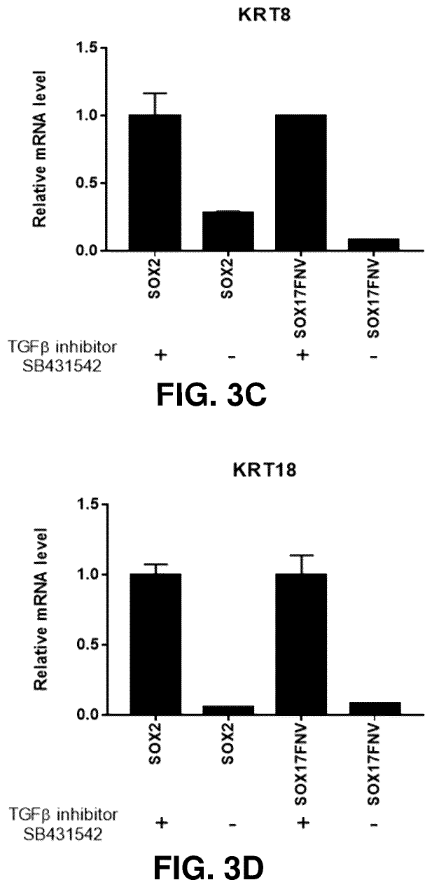

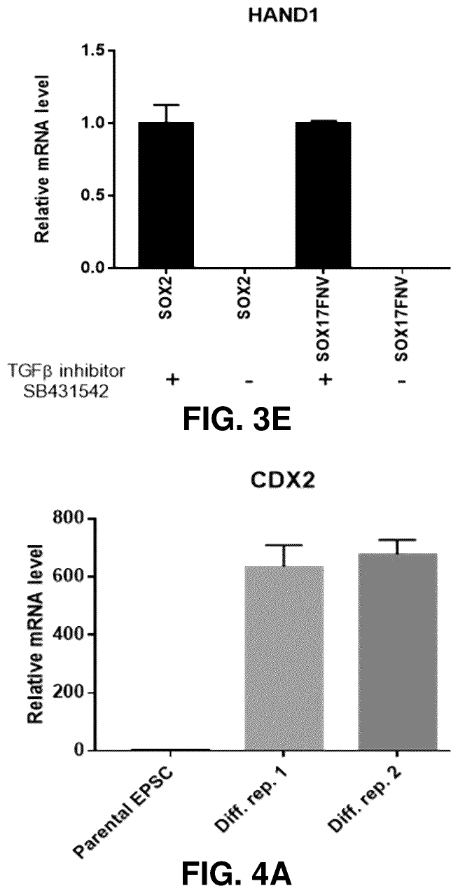

These SOX2 and SOX17FNV-derived feeder-free iESPCs was further investigated for their potential to differentiate into trophoblast lineage, which is considered as the key and unique functional characteristic of EPSCs. Treatment of TGFβ inhibitor SB431542 to EPSC has been reported to generate trophoblast-like cells (Gao, et al., Nat Cell Biol. 2019;21(6):687-99). Both SOX2 and SOX17FNV iEPSCs were demonstrated to express panels of trophoblast-related genes upon SB431542 treatment (FIG. 3A-3E).

Given the trophoblast lineage differentiation potential, trophoblast stem cells (TSCs) were then derived from the established feeder-free SOX2- and SOX17FNV-derived iEPSCs. These iEPSCs were cultured in human TSC medium as previously reported (Okae, et al., Cell Stem Cell. 2018;22(1):50-63.e6) on culture plates coated with gelatin. Colonies with TSC morphology were identified after 7-10 days with expression of pan-trophoblast marker KRT7.

### Generation of Human iBlastoids from Human iEPSCs

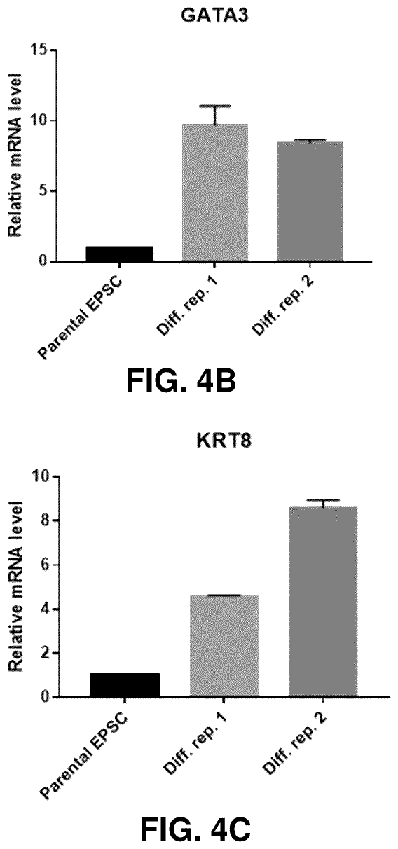

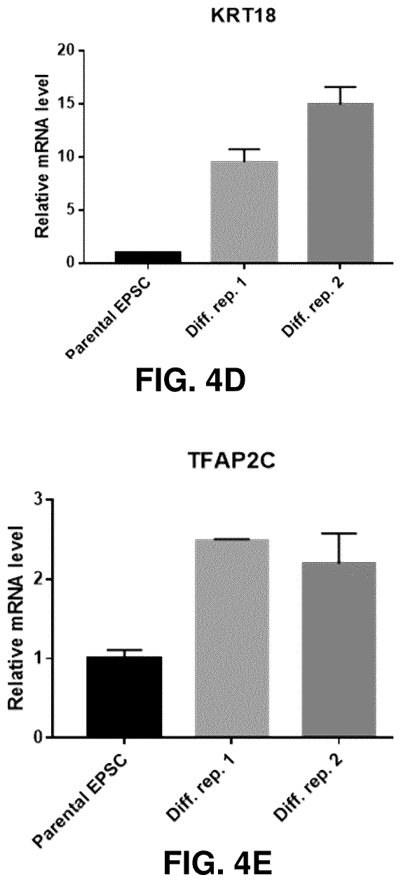

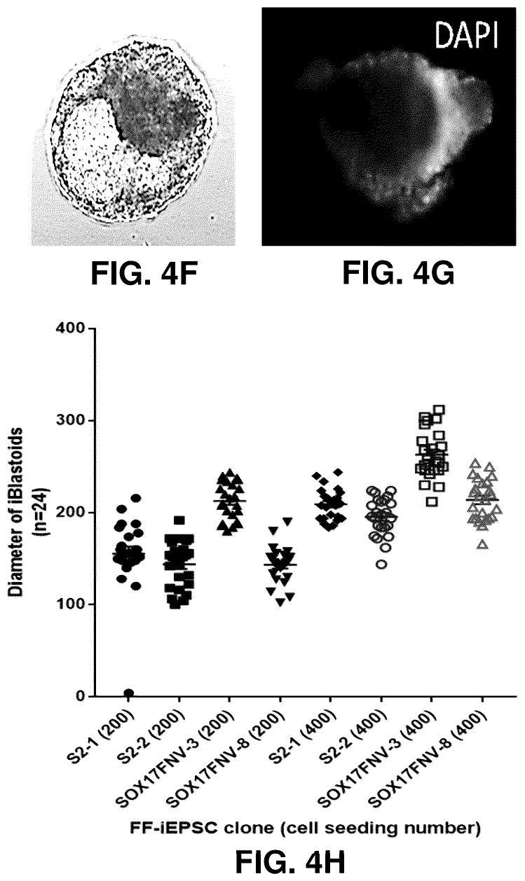

Human EPSCs exhibit more developmental potency than naive and primed pluripotent stem cells in differentiation of trophoblast lineages suggesting their totipotent-like features. It was observed that human iEPSCs express genes related to blastocytes and trophoblast lineages upon in vitro spontaneous differentiation (FIG. 4A-4E). Therefore, EPSCs were seeded in low attachment 96 well plates or microwell array and cultured in PALLY containing N2B27 medium as reported (Kagawa, et al., Nature. 2022;601(7894):600-5). Preliminary data demonstrated that some cell aggregates can form blastocyst-like structures with a cavity (FIG. 4F-4G). Size of these blastocyst-like structures varied with an average diameter 150-250 mm (FIG. 4H), similar to that in late human blastocysts (Yanagida, et al., Cell Stem Cell. 20 2021;28(6):1016-22.e4).

### Discussion

Engineered SOX17 factors are previously reported to outperform wild type SOX2 in mouse pluripotency reprogramming as well as in human iPSC reprogramming towards primed pluripotent cells. Here, it has been demonstrated that the engineered transcription factor SOX17FNV can substantially boost the speed, efficiency, and reproducibility of iEPSC reprogramming from human dermal fibroblasts compared to wild type SOX2. The resultant iEPSCs show self-renewal capacity in long term culture without losing their pluripotency features and the potential to trophoblast lineage differentiation. Human induced blastoids (iB) can be derived from these iEPSCs indicating their totipotent-like features, which might be an ideal alternative in studying human early embryo development.

Importantly, SOX17FNV enables human EPSC reprogramming in recalcitrant cells such as high-passaged senescent fibroblasts while SOX2 fails to do so. These findings indicate the remarkable potency of SOX17FNV in the translational application of human EPSCs, particularly in generation of iEPSC cell bank from fibroblasts and peripheral blood cells of individuals among different ages. Given the high efficiency of SOX17FNV, iEPSCs might be effectively and robustly generated even from rare tissue specimen such as small tissue biopsies or a few drops of blood.

The pluripotent features and trophoblast lineage differentiation potentials of SOX2- and SOX17FNV-derived human iEPSCs were also examined. These human iEPSCs demonstrated similar phenotypes as reported in EPSCs derived from human blastocysts and embryo. These reprogrammed iEPSCs can be further adapted to Matrigel or laminin to generate feeder-free and clinical-grade clones, demonstrating the potential to produce personalized EPSCs for clinical studies under GMP conditions. For the reported totipotent-like features of human EPSCs, the potency of SOX2- and SOX17FNV-derived iEPSCs in generation of induced blastoids was also examined. It was shown that both EPSCs can be induced into human iBlastoids, which are considered as an invaluable cell model in the preclinical study of infertility-related diseases such as infertility, failure of implantations and preeclampsia. The use of iEPSC for such studies would obviate the need for human embryos and usage of ethically challenging cell sources. Likewise, the basic research of early human embryo development could be boosted by an easy-to-use method of iEPSC generation that gains popularity reminiscent of primed iPSC reprogramming.

### Example 2: eSOX17^{FNV} enables reprogramming of human blood cells into iNSCs

### Methods

To apply eSox17^{FNV}-driven iNSC reprogramming to human somatic cells, peripheral blood mononuclear cells (PBMCs) were used instead of fibroblasts due to their availability via non-invasive means and a lower mutation burden compared to skin fibroblasts.

To avoid chromosome rearrangement frequently observed in lymphocytes, erythroid progenitor cells (EPCs) were expanded and used as starting cells to generate human iNSCs (hiNSCs). EPCs were transduced with Dox-inducible lentiviral particles encoding human KLF4, C-MYC and eSOX17FNV (K/M/eSOX17^{FNV}).

Representative phase-contrast microscope images of hiNSC reprogramming from human erythroid progenitor cells (EPCs) were produced and analyzed at day 20. hiNSC-like colonies were only formed in 3-factor K/M/eSOX17FNV and 2-factor M/eSOX17FNV conditions.

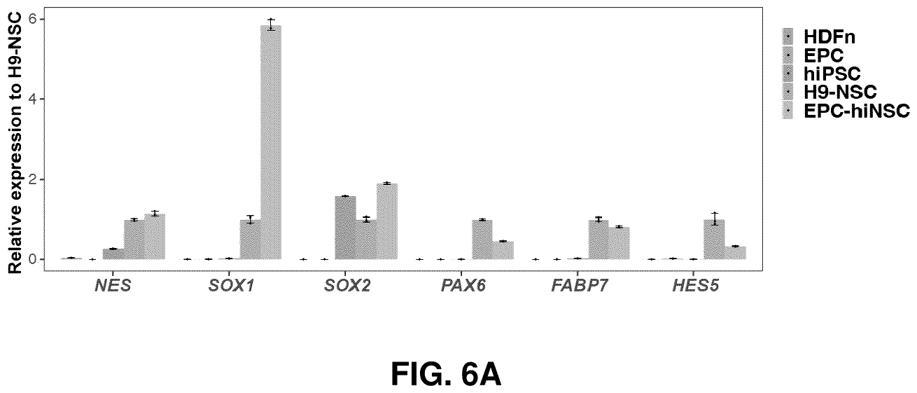

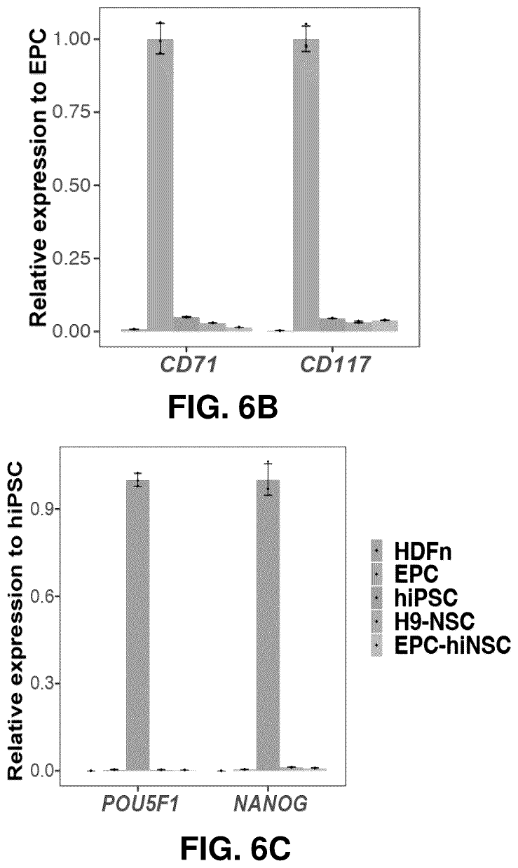

Gene expression of M/eSOX17FNV reprogrammed 2F-hiNSCs was detected by qRT-PCR. Neonatal human dermal fibroblasts (HDFn), EPCs, hiPSCs and H9-NSCs were used as controls. Expression of NSC genes was normalized to H9-NSCs, EPC genes was normalized to EPCs, and pluripotency genes was normalized to hiPSCs (see FIGS. 6A-6C).

After 3 weeks of in vitro spontaneous differentiation, hiNSCs gave rise to NF+/TH+ dopaminergic neurons and MAP2+/vGLUT2+ glutamatergic neurons.

After 2 weeks of in vitro motor neuron differentiation, hiNSCs gave rise to TUJ1+/HB9+ and NF+/ISL1+ motor neurons. Neurons spontaneously differentiated from hiNSCs were immunoreactive for MAP2, SYN1 and PSD95.

Normalized Ca2+ Fluo-4 traces of fluorescence live-cell imaging in 5 individual spontaneously differentiated neurons from hiNSCs revealed spontaneous neural network activity.

Gene ontology (GO) analysis were performed using top 300 up-regulated DEGs in hiNSCs compared with EPCs.

### Results

It was found that overexpression of K/M/eSOX17^{FNV} and culture in an optimized human NSCs medium with bFGF, EGF, CHIR99021 (GSK-3 inhibitor), SB431542 (TGFβRI/ALK5 inhibitor), purmorphamine (hedgehog-smoothened (Shh) agonist), tranylcypromine (monoamine oxidase (MAO) inhibitor), and sodium butyrate (histone deacetylase (HDAC) inhibitor) allows the conversion of EPCs into iNSCs (FIG. 5A). After omitting individual factors from this 3-factor cocktail, it was found that C-MYC and eSOX17^{FNV} (M/eSoX17^{FNV}) are sufficient for iNSC conversion. Transduced EPCs formed adherent, elongated cells within 4 days and NSC-like colonies within 11-20 days. These colonies can be picked and expanded in hNSC medium supplemented with bFGF, EGF, CHIR99021, and SB431542. Stable hiNSCs independent of transgenes could be generated after 5 passages. Immunocytochemistry of hiNSCs using antibodies against SOX2, PAX6, SOX1, FABP7 revealed that hiNSCs express NSC markers SOX1, SOX2, PAX6 and FABP7 (BLBP) (FIG. 6A), but EPC markers CD71 (TFRC) and CD117 (KIT) or pluripotency genes POU5F1 and NANOG could not be detected (FIGS. 6B and 6C). Representative karyograms of hiNSCs from EPCs of donor 275-5 at passage indicated that hiNSCs could be stably expanded on matrigel in hNSC medium for more than 20 passages with a normal karyotype.

To evaluate the multipotency of M/eSOX17^{FNV} reprogrammed hiNSCs, hiNSCs were differentiated in various conditions. In a spontaneous neuronal differentiation condition within 3 weeks, hiNSCs gave rise to NF⁺/TH⁺ dopaminergic neurons and MAP2⁺/vGLUT2⁺ glutamatergic neurons, respectively. hiNSCs could also quickly differentiate into TUJ1⁺/HB9⁺ and NF⁺/ISL1⁺ motor neurons within 2 weeks. Notably, hiNSC-derived neurons were immunoreactive for presynaptic marker Synapsin-1 (SYN1) and postsynaptic marker PSD-95. These neurons (12 out of 34) displayed frequent and spontaneous neuronal activity detected by calcium imaging, indicating the generation of mature and functional neurons. In addition to neuronal lineages, hiNSCs were also able to differentiate into GFAP⁺ astrocytes and O4⁺/OLIG2⁺ oligodendrocytes in vitro, as determined by immunofluorescence microscopy. These results indicated that EPC-hiNSCs can generate multiple neuronal subtypes and glial cells.

To further characterize the hiNSCs, the global gene expression of EPCs, three hiNSCs lines from two donors, ESCs, hiPSCs and ESC/hiPSC-derived neural progenitor cells (NPCs) were analyzed. PC analysis of global gene expression profiles determined by bulk RNA-seq revealed that hiNSCs clustered closely with ESC/hiPSC-derived NPCs but are distinct from EPC and PSC clusters (FIG. 5B). Gene ontology (GO) analysis of the top 300 upregulated genes in hiNSCs compared with EPCs unveiled an enrichment for neural related biological processes, such as axon development, axonogenesis and forebrain development (FIG. 5C). Analysis of mean expression of selected NSC, PSC and EPC lineage markers represented as log2 transformed read counts in hiNSCs indicated that many NSC markers such as SOX1, SOX2, PAX6, FABP7 and HES5 were highly expressed, while EPC markers such as GYPA, HBA1 and IKZF1 and pluripotency markers such as POU5F1, NANOG and ZFP42 were repressed (FIG. 5D).

The regional identity of hiNSCs was also investigated by analyzing mean expression of selected forebrain, midbrain, hindbrain, spinal cord, dorsal and ventral regional identity genes represented as log2 transformed read counts. hiNSCs expressed forebrain markers (SIX3 and DLX2), midbrain marker (PAX5, EN1 and EN2), hindbrain markers (GBX2, HOXB2 AND HOXB4) but not spinal cord markers (HOXB5, HOXB6, HOXB7 and HOXC6) (FIG. 5E). Along the dorso-ventral axis, hiNSCs expressed several dorsal markers such as PAX3, IRX3, IRX5, and SP8 while ventral markers were poorly expressed. These results suggest that hiNSCs have a dorso-anterior identity. Together, the data demonstrated that eSOX17^{FNV} can generate self-renewing and multipotent iNSCs directly from human blood.

### Discussion

It has been demonstrated that engineered transcription factors can facilitate the direct conversion of somatic cells into iNSCs. Dox-inducible lentivirus was used instead of a constitutive retrovirus to control exogenous gene expression over a smaller number of starting cells and shorter reprogramming time frame, using purified Sox2-GFP MEFs rather than bulk cells without reporter as starting material. To ascertain that engineered Sox17 is imparts direct lineage reprogramming, Sox2 and Sox17 were used as controls. Clearly, the changed capacity to bind partner factors including but not limited to Brn2 affects DNA and chromatin interactions and alters the mode as to how eSox17FNV explores genomic DNA as shown by single-molecule tracking. Compared to Sox2, Sox17 possess a unique C-terminal domain that potently promotes iNSC generation. During transgene overexpression, subtly different gene expression profiles and chromatin opening were observed between eSox17FNV and Sox2/Sox17. Once transgenes are silenced, profound differences emerge and only cells previously exposed to eSox17FNV mature towards a neural stem cell state, indicating that eSox17FNV creates a poised state through altered genome engagement and co-factor recruitment but needs to give way to endogenous factors for the lineage commitment to take place. This is in stark contrast to iPSC reprogramming where active transgenes do not impede the activation of pluripotency network.

Taken together, this study presents a robust approach to reprogram murine and human somatic cells into iNSCs with eSox17FNV.

We claim:

1. A method for reprogramming human somatic cells into human induced totipotent or multipotent stem cells, the method comprising:
(i) introducing one or more exogenous transcription factors to the human somatic cells to provide reprogrammed cells,
wherein one exogenous transcription factor comprises an engineered SOX17 factor (eSOX17), and
(ii) culturing the reprogrammed cells in a stem cell medium to provide induced totipotent or multipotent cells, and
(iii) optionally isolating the induced totipotent or multipotent stem cells.

2. The method of claim 1, wherein the eSOX17 comprises the amino acid sequence of SEQ ID NO:1, or an amino acid sequence comprising at least 70%, 75%, 80%, 85%, 90%, 95%, or 99% sequence identity to SEQ ID NO:1, or a fragment thereof.

3. The method of claim 1, wherein the eSOX17 comprising the amino acid sequence of SEQ ID NO:2, or an amino acid sequence having at least 70%, 75%, 80%, 85%, 90%, 95%, or 99% sequence identity to SEQ ID NO:2, or a fragment variant thereof.

4. The method of claim 1, wherein the eSOX17 comprises the amino acid sequence of any one of SEQ ID NOs:1-2 or 4-54.

5. The method of claim 1, wherein the one or more exogenous transcription factors further comprises one or more of OCT4, KLF4, and C-MYC, and
wherein the stem cell medium comprises an expanded potential stem cell medium (EPSCM).

6. The method of claim 1, wherein the eSOX17 comprises the amino acid sequence of SEQ ID NO:1 or SEQ ID NO:2,
wherein the exogenous transcription factors further comprise one or more of KLF4 and C-MYC, and
wherein the stem cell medium comprises a neural stem cell medium (NSCM).

7. The method of claim 1, wherein the one or more exogenous transcription factors are encoded as nucleic acids, optionally wherein the nucleic acids are encoded and/or expressed by a vector.

8. The method of claim 7, wherein the vector is an integration-free system selected from the group consisting of episomal vectors, minicircle vectors, and Sendai viral vectors.

9. The method of claim 7, wherein the vector is a viral vector selected from the group consisting of retrovirus, lentivirus, adenovirus, or herpes virus.

10. The method of claim 5, wherein the EPSCM comprises one or more of a RAS-ERK inhibitor, a Src Kinase family (SFK) inhibitor, a GSK3 inhibitor, a Wnt inhibitor, a p38 inhibitor, and a JNK inhibitor.

11. The method of claim 1, wherein the induced totipotent or multipotent stem cells are isolated in less than 8 days, less than 7 days, less than 6 days, less than 5 days, or less than 4 days after culturing in stem cell medium.

12. The method of claim 1, wherein culturing the reprogrammed cells comprises culturing in the absence of feeder cells.

13. The method of claim 1 further comprising expanding the induced totipotent or multipotent stem cells for at least 20 passages and up to 42 passages in cell culture.

14. The method of claim 1 further comprising, after step (ii),
culturing the induced totipotent cells in a trophoblast induction medium to induce differentiation of the cells into trophoblasts, or
culturing the induced totipotent cells in a blastoid induction medium to induce differentiation of the cells into blastoids.

15. The method of claim 1, wherein the human somatic cells are selected from the group consisting of cells of hematological origin, skin derived cells, adipose cells, epithelial cells, endothelial cells, cells of mesenchymal origin, parenchymal cells, neurological cells, and connective tissue cells.

16. The method of claim 15, wherein the human somatic cells are dermal fibroblasts or peripheral blood cells.

17. Human induced expanded potential stem cells (iEPSCs) obtained by the method of claim 5, optionally wherein the iEPSCs are frozen in a cryo-preservative.

18. Human induced Neural Stem Cells (iNSC) obtained by the method of claim 6, optionally wherein the iNSCs are frozen in a cryo-preservative.

19. A method of using the induced totipotent or multipotent cells produced by the method of claim 1 for treating or preventing one or more disease or disorders in a subject in need thereof,
optionally wherein the one or more disease or disorders are selected from the group consisting of wounds, injuries, cancers, diabetes, and neurodegenerative diseases.

20. An engineered SOX17 factor (eSOX17) comprising:
(i) the amino acid sequence of any one of SEQ ID NO:1-2 or SEQ ID NO:4-54, or an amino acid sequence comprising at least 70%, 75%, 80%, 85%, 90%, 95%, or 99% sequence identity to SEQ ID NO:1-2, or SEQ ID NO:4-54, or a fragment variant thereof; or
(ii) a nucleic acid encoding a polypeptide comprising the amino acid sequence of any one of SEQ ID NO:1-2 or SEQ ID NO:4-54, or an amino acid sequence comprising at least 70%, 75%, 80%, 85%, 90%, 95%, or 99% sequence identity to SEQ ID NO:1-2, or SEQ ID NO:4-54, or a fragment variant thereof.
